(19) United States
(12) Patent Application Publication
(10) Pub. No.: US 2007/0185066 A1
(43) Pub. Date: Aug. 9, 2007

(54) Systems and methods for the delivery of corticosteroids

(75) Inventor: Malcolm Hill, Solana Beach, CA (US)

Correspondence Address: WILSON SONSINI GOODRICH & ROSATI, 650 PAGE MILL ROAD, PALO ALTO, CA, 94304-1050, US

(73) Assignee: Verus Pharmaceuticals, Inc., San Diego, CA (US)

(21) Appl. No.: 11/643,638

(22) Filed: Dec. 19, 2006

Related U.S. Application Data
(60) Provisional application No. 60/752,735, filed on Dec. 20, 2005.
(60) Provisional application No. 60/773,998, filed on Feb. 15, 2006.
(60) Provisional application No. 60/773,999, filed on Feb. 15, 2006.
(60) Provisional application No. 60/820,092, filed on Jul. 21, 2006.
(60) Provisional application No. 60/820,094, filed on Jul. 21, 2006.
(60) Provisional application No. 60/820,096, filed on Jul. 21, 2006.

(51) Int. Cl. A61K 31/56

(52) U.S. Cl. 514/170; 514/169

(57) ABSTRACT

The present invention relates to methods and systems for the delivery of a corticosteroid comprising (1) an inhalable aqueous mixture comprising a corticosteroid and a solubility enhancer and (2) an inhalable nebulizer, wherein the delivery of the aqueous mixture comprising the corticosteroid by the nebulizer results in an enhanced pharmacokinetic profile of the corticosteroid as compared to conventional inhalable therapies and/or increase lung deposition.

### FIELD OF THE INVENTION

The present invention relates to methods and systems for the delivery of a corticosteroid comprising (1) an inhalable aqueous mixture comprising a corticosteroid and a solubility enhancer and (2) an inhalable nebulizer, wherein the delivery of the aqueous mixture comprising the corticosteroid by the nebulizer results in an enhanced pharmacokinetic profile of the corticosteroid as compared to conventional inhalable therapies and/or increased lung deposition.

### BACKGROUND OF THE INVENTION

Inhaled corticosteroids are fundamental to the long-term management of persistent asthma and are recommended by national guidelines for therapy of young children diagnosed with asthma. Numerous clinical trials support their efficacy and relative safety for children. In addition, it is believed that early corticosteroid intervention can play a critical role in the reduction of permanent lung damage and alter the chronic, progressive nature of the disease.

The use of inhaled corticosteroids in the treatment of asthma provides significant benefit due to the direct delivery to the site of action, the lung (as used herein, “lung” refers to either or both the right and left lung organs). The goal of inhaled corticosteroid therapy is to provide localized delivery of the corticosteroid with immediate drug activity at the site of action. It is known that inhaled corticosteroids are well absorbed from the lungs. In fact, it can be assumed that substantially all of the drug available at the receptor site in the lungs will be absorbed. However, it is also known that current methods and formulations result in a greater part of an inhaled corticosteroid dose being swallowed and becoming available for oral absorption. Thus, due to the particular method or system employed, some corticosteroids are more likely to be deposited in the mouth and throat than the lungs, and may cause adverse effects. For the portion of the inhaled corticosteroid dose delivered orally, bioavailability depends upon absorption from the GI tract and the extent of first pass metabolism in the liver. Since this oral component of corticosteroid drug delivery does not provide any beneficial therapeutic effect and increases the risk of systemic side effects, it is desirable for the oral bioavailability of inhaled corticosteroid to be relatively low. Thus, for inhaled corticosteroids, high pulmonary availability is more important than high oral bioavailability because the lung is the target organ.

As such, a method or system of delivery that provides a corticosteroid with high pulmonary availability has greater potential to exert positive effects in the lung. The ideal system of providing inhaled corticosteroids would provide minimum oral delivery and reduced administration times thereby reducing the likelihood of systemic adverse effects.

Unfortunately, however, the delivery of a corticosteroid via inhalation often results in deposition of the corticosteroid in sections distinct from the respiratory tract, e.g., mouth, throat, and esophagus. Generally, the smaller the particle size of the corticosteroid, the longer the particle will remain suspended in air and the farther down the respiratory tract the drug can be delivered. Corticosteroids are delivered by inhalation using nebulizers, metered dose inhalers, or dry powder inhalers. The principle advantage of nebulizers over other methods of pulmonary delivery of a corticosteroid is that nebulizers can more efficiently deliver higher doses of medication compared to other methods. The main concerns about nebulizers, however, are the increased cost, reduced portability, and the inconvenience of needing to prepare medication beforehand and the increased time requirement for administering a treatment. Thus, a method of improving the delivery of drugs, such as corticosteroids by nebulization, is desired.

Both particle size and formulation influence the efficacy of an inhaled corticosteroid. The formulation of a drug has a significant impact on the delivery of that drug to the lungs, and therefore its efficacy. Additionally, it is believed that the most important considerations in the delivery of drug to the lung are the aerosol vehicle and the size of the particles delivered.

The inhalation of drug particles as opposed to dissolved drug is known to be disadvantageous. Brain et al. (Bronchial Asthma, 2nd Ed. (Ed. E. B. Weis et al., Little Brown & Co. (1985), pp. 594-603) report that less soluble particles that deposit on the mucous blanket covering pulmonary airways and the nasal passages are moved toward the pharynx by the cilia. Such particles include larger drug particles which are deposited in the upper respiratory tract. Mucus, cells and debris corning from the nasal cavities and the lungs meet at the pharynx, mix with saliva, and enter the gastrointestinal tract upon being swallowed. Reportedly, by this mechanism, particles are removed from the lungs with half-times of minutes to hours. Accordingly, there is little time for solubilization of slowly dissolving drugs, including corticosteroids, e.g., budesonide. In contrast, particles deposited in the non-ciliated compartments, such as the alveoli, have much longer residence times. Since it is difficult to generate very small particles of corticosteroids for deep lung deposition, much of the inhaled suspension would likely be found in the upper to middle respiratory tract. However, it is much easier to generate small droplets from a solution than it is from a suspension of solids.

Budesonide (R,S)-11β,16α,17,21-tetrahydroxypregna-1,4-diene-3,20-dione cyclic 16,17-acetal with butyraldehyde, (C₂₅H₃₄O₆; MW: 430.5) is employed in particular for the treatment of bronchial disorders. Budesonide is a racemate consisting of a mixture of the two diastereomers 22R and 22S and is provided commercially as a mixture of the two isomers (22R and 22S). It acts as an anti-inflammatory corticosteroid that exhibits potent glucocorticoid activity. Administration of budesonide is indicated for maintenance treatment of asthma and as prophylactic therapy in children.

Because of its lipophilicity, budesonide, as well as other lipophilic corticosteroids, is virtually insoluble in water but is readily soluble in alcohols. An adequate amount of active substance can be dissolved by the use of solubilizers such as organic, water-soluble alcohols. However, the solutions obtained in this way generally limited stability for pharmaceutical use because large amounts of the active substance may decompose within a short time.

Commercial formulations of budesonide are sold by AstraZeneca LP (Wilmington, Del.) under the trademarks Entocort® EC, Pulmicort Respules®, Rhinocort® Aqua, Rhinocort® Nasal Inhaler and Pulmicort® Turbuhaler, and under its generic name. Pulmicort Respules®, which is a sterile aqueous suspension of micronized budesonide, is administered by inhalation using a nebulizer, in particular a compressed air driven jet nebulizer. Rhinocort® Nasal Inhaler is a metered-dose pressurized aerosol unit containing a suspension of micronized budesonide in a mixture of propellants. Rhinocort® Aqua is an unscented metered-dose manual-pump spray formulation containing a suspension of micronized budesonide in an aqueous medium. In addition, suspension formulations of budesonide have a propensity to rapidly form coarse flocs upon dispersion and re-dispersion which may deleteriously affect dosage reproducibility. There is also a tendency for budesonide to deposit from suspension onto the walls of the container.

Accordingly, there is a need for systems and methods for delivering a non-suspension formulation comprising a corticosteroid by nebulization. However, even in light of this need, the Pulmicort Respule® suspension is the only currently approved therapy for the treatment of pediatric asthma with budesonide via inhalation therapy. In addition, the availability of compositions, methods, and systems for corticosteroids other than budesonide is likewise needed. Thus, it would be a significant advancement to the field of corticosteroid inhalation therapy to provide a method or system which provides enhanced pharmacokinetic profiles of the delivered corticosteroid as compared to the pharmacokinetic profile of suspension unit dose formulations containing a corticosteroid, and/or increased lung deposition.

### SUMMARY OF THE INVENTION

In certain embodiments, the present invention provides an inhalable composition comprising about 250 μg or less of a single corticosteroid, a solvent and a solubility enhancer, wherein upon administration of the composition to a subject through a device, the composition achieves a rate of increasing concentration of the corticosteroid inside the device of about 5 μg/ml per minute or less over administration of the corticosteroid through the device and wherein the composition is substantially free of active pharmaceutical agents other than the corticosteroid.

In certain embodiments, the present invention provides an inhalable composition wherein the composition achieves a rate of increasing concentration of the corticosteroid inside the device of about 5 μg/ml per minute or less over the first three minutes of administration.

In certain embodiments, the present invention provides an inhalable composition, wherein the composition achieves a rate of increasing concentration of the corticosteroid inside the device of about 3.5 μg/nm per minute or less over the first 3 minutes of administration.

In certain embodiments, the present invention provides an inhalable composition wherein the composition achieves a rate of increase in concentration of the corticosteroid inside the device of about 5% per minute or less over the first 3 minutes of administration.

In certain embodiments, the present invention provides an inhalable composition wherein the solvent comprises water.

In certain embodiments, the present invention provides an inhalable composition wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodexthn, maltosyl-β-cyclodextrin, maltosyl-β-cyclodextrin, maltotriosyl-γ-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer comprises SBE7-β-CD.

In certain embodiments, the present invention provides an inhalable composition wherein the device is a nebulizer. In certain embodiments, the device is a Pari eFlow nebulizer.

In certain embodiments, the present invention provides an inhalable composition wherein the composition comprises the corticosteroid in a nominal dosage of about 60 μg.

In certain embodiments, the present invention provides an inhalable composition wherein the composition comprises the corticosteroid in a nominal dosage of about 120 μg.

In certain embodiments, the present invention provides an inhalable composition comprising an effective amount of a single corticosteroid, a solvent and a solubility enhancer, wherein upon administration of the composition to a subject through a device, the composition achieves a rate of increasing concentration of the corticosteroid inside the device of 60% or less of a rate of increasing concentration of the corticosteroid inside the device achieved by an inhalable suspension comprising the corticosteroid without a solubility enhancer administered under the same conditions and wherein the composition is substantially free of active pharmaceutical agents other than the corticosteroid.

In certain embodiments, the present invention provides an inhalable composition wherein the rate of increasing concentration of the corticosteroid inside the device is achieved over the first 3 minutes, during the second and third minutes or during the third minute of administration.

In certain embodiments, the present invention provides an inhalable composition, wherein administration of the composition through the device is achieved over five minutes or less, and administration of the inhalable suspension is achieved over five minutes or less.

In certain embodiments, the present invention provides an inhalable composition, wherein the time of administration of the composition through the device and the time of administration of the inhalable suspension are the same.

In certain embodiments, the present invention provides an inhalable composition wherein the time of administration of the composition through the device and the time of administration of the inhalable suspension are different.

In certain embodiments, the present invention provides an inhalable composition wherein the composition comprises about 40, about 60, about 120, about 125, about 240, about 250, about 500, about 1000, about 1500, or about 2000 μg of the corticosteroid.

In certain embodiments, the present invention provides an inhalable composition wherein the solvent comprises water.

In certain embodiments, the present invention provides an inhalable composition wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides an inhalable composition, wherein the solubility enhancer comprises SBE7-β-CD.

In certain embodiments, the present invention provides an inhalable composition wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides a method of generating fine particles from an inhalable composition comprising: forming the composition by adding a solvent and a solubility enhancer to an effective amount of a single corticosteroid, wherein the composition is substantially free of active pharmaceutical agents other than the corticosteroid and, and operating a nebulizer to produce fine particles of the composition, wherein upon administration of the composition to a subject through the nebulizer, the composition achieves a rate of increasing concentration of the corticosteroid inside the nebulizer of 60% or less of a rate of increasing concentration of the corticosteroid inside the nebulizer achieved by an inhalable suspension comprising the corticosteroid without a solubility enhancer administered under the same conditions.

In certain embodiments, the present invention provides a method, wherein the rate of increasing concentration of the corticosteroid inside the device is achieved over the first 3 minutes, during the second and third minutes or during the third minute of administration.

In certain embodiments, the present invention provides a method wherein administration of the composition through the nebulizer is achieved over five minutes or less, and administration of the inhalable suspension is achieved over five minutes or less.

In certain embodiments, the present invention provides a method wherein the time of administration of the composition through the nebulizer and the time of administration of the inhalable suspension are the same.

In certain embodiments, the present invention provides a method, wherein the time of administration of the composition through nebulizer and the time of administration of the inhalable suspension are different.

In certain embodiments, the present invention provides a method wherein the composition comprises about 40, about 60, about 120, about 125, about 240, about 250, about 500, about 1000, about 1500, or about 2000 μg of the corticosteroid.

In certain embodiments, the present invention provides a method wherein the solvent comprises water.

In certain embodiments, the present invention provides a method wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl, β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dilydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides a method wherein the solubility enhancer comprises SBE7-β-CD.

In certain embodiments, the present invention provides a method, wherein the nebulizer is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides an inhalation system for delivering a therapeutically effective dose of a single corticosteroid to a patient comprising: (a) an aqueous inhalation mixture comprising the corticosteroid, a solvent and a solubility enhancer, wherein the composition is substantially free of active pharmaceutical agents other than the corticosteroid and and (b) a nebulizer wherein upon administration of the mixture to a subject through the nebulizer, the mixture achieves a rate of increasing concentration of the corticosteroid inside the nebulizer of 60% or less of a rate of increasing concentration of the corticosteroid inside the nebulizer achieved by an inhalable suspension comprising the corticosteroid without a solubility enhancer administered under the same conditions.

In certain embodiments, the present invention provides an inhalation system wherein the rate of increasing concentration of the corticosteroid inside the device is achieved over the first 3 minutes, during the second and third minutes or during the third minute of administration.

In certain embodiments, the present invention provides an inhalation system wherein administration of the composition through the nebulizer is achieved over five minutes or less, and administration of the inhalable suspension is achieved over five minutes or less.

In certain embodiments, the present invention provides an inhalation system, wherein the time of administration of the composition through the nebulizer and the time of administration of the inhalable suspension are the same.

In certain embodiments, the present invention provides an inhalation system wherein the time of administration of the composition through the nebulizer and the time of administration of the inhalable suspension are different.

In certain embodiments, the present invention provides an inhalation system wherein the mixture comprises about 40, about 60, about 120, about 125, about 240, about 250, about 500, about 1000, about 1500, or about 2000 μg of the corticosteroid.

In certain embodiments, the present invention provides an inhalation system wherein the solvent comprises water.

In certain embodiments, the present invention provides an inhalation system wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl, β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides an inhalation system wherein the solubility enhancer comprises SBE7-β-CD.

In certain embodiments, the present invention provides an inhalation system wherein the nebulizer is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides a method for the treatment of a bronchoconstrictive disorder in a patient in need of treatment thereof, comprising: forming a composition by adding a solvent and a solubility enhancer to an amount of a single corticosteroid, wherein the composition is substantially free of active pharmaceutical agents other than the corticosteroid and and operating a nebulizer, wherein upon administration of the composition to a subject through the nebulizer, the composition achieves a rate of increasing concentration of the corticosteroid inside the nebulizer of 60% or less of a rate of increasing concentration of the corticosteroid inside the nebulizer achieved by an inhalable suspension comprising the corticosteroid without a solubility enhancer administered under the same conditions.

In certain embodiments, the present invention provides a method wherein the rate of increasing concentration of the corticosteroid inside the device is achieved over the first 3 minutes, during the second and third minutes or during the third minute of administration.

In certain embodiments, the present invention provides a method wherein administration of the composition through the nebulizer is achieved over five minutes or less, and administration of the inhalable suspension is achieved over five minutes or less.

In certain embodiments, the present invention provides a method wherein the time of administration of the composition through the nebulizer and the time of administration of the inhalable suspension are the same.

In certain embodiments, the present invention provides a method wherein the time of administration of the composition through the nebulizer and the time of administration of the inhalable suspension are different.

In certain embodiments, the present invention provides a method, wherein the composition comprises about 40, about 60, about 120, about 125, about 240, about 250, about 500, about 1000, about 1500, or about 2000 μg of the corticosteroid.

In certain embodiments, the present invention provides a method wherein the solvent comprises water.

In certain embodiments, the present invention provides a method wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides a method, wherein the solubility enhancer comprises SBE7-β-CD.

In certain embodiments, the present invention provides a method, wherein the nebulizer is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides a method wherein the bronchoconstrictive disorder is selected from the group consisting of asthma, pediatric asthma, bronchial asthma, allergic asthma, intrinsic asthma, chronic obstructive pulmonary disease (COPD), chronic bronchitis, and emphysema.

In certain embodiments, the present invention provides an inhalable composition comprising about 250 μg or less of budesonide, a solvent and a solubility enhancer, wherein upon administration of the composition to a subject through a device, the composition achieves a rate of increasing concentration of budesonide inside the device of about 5 μg/ml per minute or less over administration of the budesonide through the device and wherein the composition is substantially free of active pharmaceutical agents other than the corticosteroid.

In certain embodiments, the present invention provides an inhalable composition, wherein the composition achieves a rate of increasing concentration of the budesonide inside the device of about 5 μg/ml per minute or less over the first three minutes of administration.

In certain embodiments, the present invention provides an inhalable composition, wherein the composition achieves a rate of increasing concentration of budesonide inside the device of about 3.5 μg/ml per minute or less over the first 3 minutes of administration.

In certain embodiments, the present invention provides an inhalable composition, wherein the composition achieves a rate of increase in concentration of budesonide inside the device of about 5% per minute or less over the first 3 minutes of administration.

In certain embodiments, the present invention provides an inhalable composition, wherein the solvent comprises water.

In certain embodiments, the present invention provides an inhalable composition wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl, β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof

In certain embodiments, the present invention provides an inhalable composition, wherein the solubility enhancer comprises SBE7-β-CD.

In certain embodiments, the present invention provides an inhalable composition, wherein the device is a nebulizer.

In certain embodiments, the present invention provides an inhalable composition wherein the device is a Pari eFlow nebulizer.

In certain embodiments, the present invention provides an inhalable composition the composition comprises budesonide in a nominal dosage of about 60 μg.

In certain embodiments, the present invention provides an inhalable composition the composition comprises budesonide in a nominal dosage of about 120 μg.

In certain embodiments, the present invention provides an inhalable composition comprising an effective amount of budesonide, a solvent and a solubility enhancer, wherein upon administration of the composition to a subject through a device, the composition achieves a rate of increasing concentration of budesonide inside the device of 60% or less of a rate of increasing concentration of budesonide inside the device achieved by an inhalable suspension comprising budesonide without a solubility enhancer administered under the same conditions and wherein the composition is substantially free of active pharmaceutical agents other than the corticosteroid.

In certain embodiments, the present invention provides an inhalable composition wherein the rate of increasing concentration of budesonide inside the device is achieved over the first 3 minutes, during the second and third minutes or during the third minute of administration.

In certain embodiments, the present invention provides an inhalable composition wherein administration of the composition through the device is achieved over five minutes or less, and administration of the inhalable suspension is achieved over five minutes or less.

In certain embodiments, the present invention provides an inhalable composition wherein the time of administration of the composition through the device and the time of administration of the inhalable suspension are the same.

In certain embodiments, the present invention provides an inhalable composition wherein the time of administration of the composition through the device and the time of administration of the inhalable suspension are different.

In certain embodiments, the present invention provides an inhalable composition wherein the composition comprises about 40, about 60, about 120, about 125, about 240, about 250, about 500, about 1000, about 1500, or about 2000 μg of budesonide.

In certain embodiments, the present invention provides an inhalable composition wherein the solvent comprises water.

In certain embodiments, the present invention provides an inhalable composition wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl, β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides an inhalable composition, wherein the solubility enhancer comprises SBE7-β-CD.

In certain embodiments, the present invention provides an inhalable composition wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides a method of generating fine particles from an inhalable composition comprising: forming the composition by adding a solubility enhancer to an effective amount of budesonide, wherein the composition is substantially free of active pharmaceutical agents other than the corticosteroid, and operating a nebulizer to produce fine particles of the composition, wherein upon administration of the composition to a subject through the nebulizer, the composition achieves a rate of increasing concentration of budesonide inside the nebulizer of 60% or less of a rate of increasing concentration of budesonide inside the nebulizer achieved by an inhalable suspension comprising budesonide without a solubility enhancer administered under the same conditions.

In certain embodiments, the present invention provides a method, wherein the rate of increasing concentration of budesonide inside the device is achieved over the first 3 minutes, during the second and third minutes or during the third minute of administration.

In certain embodiments, the present invention provides a method, wherein administration of the composition through the nebulizer is achieved over five minutes or less, and administration of the inhalable suspension is achieved over five minutes or less.

In certain embodiments, the present invention provides a method, wherein the time of administration of the composition through the nebulizer and the time of administration of the inhalable suspension are the same.

In certain embodiments, the present invention provides a method, wherein the time of administration of the composition through nebulizer and the time of administration of the inhalable suspension are different.

In certain embodiments, the present invention provides a method, wherein the composition comprises about 40, about 60, about 120, about 125, about 240, about 250, about 500, about 1000, about 1500, or about 2000 μg of budesonide.

In certain embodiments, the present invention provides a method, wherein the solvent comprises water.

In certain embodiments, the present invention provides a method, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides a method, wherein the solubility enhancer comprises SBE7-β-CD.

In certain embodiments, the present invention provides a method, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides an inhalation system for delivering a therapeutically effective dose of budesonide to a patient comprising: (a) an aqueous inhalation mixture comprising budesonide and a solubility enhancer, and wherein the composition is substantially free of active pharmaceutical agents other than the corticosteroid and (b) a nebulizer wherein upon administration of the mixture to a subject through the nebulizer, the mixture achieves a rate of increasing concentration of budesonide inside the nebulizer of 60% or less of a rate of increasing concentration of budesonide inside the nebulizer achieved by an inhalable suspension comprising budesonide without a solubility enhancer administered under the same conditions.

In certain embodiments, the present invention provides an inhalation system, wherein the rate of increasing concentration of budesonide inside the device is achieved over the first 3 minutes, during the second and third minutes or during the third minute of administration.

In certain embodiments, the present invention provides an inhalation system. The system of claim 84, wherein administration of the composition through the nebulizer is achieved over five minutes or less, and administration of the inhalable suspension is achieved over five minutes or less.

In certain embodiments, the present invention provides an inhalation system, wherein the time of administration of the composition through the nebulizer and the time of administration of the inhalable suspension are the same.

In certain embodiments, the present invention provides an inhalation system, wherein the time of administration of the composition through the nebulizer and the time of administration of the inhalable suspension are different.

In certain embodiments, the present invention provides an inhalation system, wherein the mixture comprises about 40, about 60, about 120, about 125, about 240, about 250, about 500, about 1000, about 1500, or about 2000 μg of budesonide.

In certain embodiments, the present invention provides an inhalation system, wherein the solvent comprises water.

In certain embodiments, the present invention provides an inhalation system, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides an inhalation system, wherein the solubility enhancer comprises SBE7-β-CD.

In certain embodiments, the present invention provides an inhalation system, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides a method for the treatment of a bronchoconstrictive disorder in a patient in need of treatment thereof, comprising: forming a composition by adding a solubility enhancer to an amount of budesonide, wherein the composition is substantially free of active pharmaceutical agents other than the corticosteroid and operating a nebulizer, wherein upon administration of the composition to a subject through the nebulizer, the composition achieves a rate of increasing concentration of budesonide inside the nebulizer of 60% or less of a rate of increasing concentration of budesonide inside the nebulizer achieved by an inhalable suspension comprising budesonide without a solubility enhancer administered under the same conditions.

In certain embodiments, the present invention provides a method, wherein the rate of increasing concentration of budesonide inside the device is achieved over the first 3 minutes, during the second and third minutes or during the third minute of administration.

In certain embodiments, the present invention provides a method wherein administration of the composition through the nebulizer is achieved over five minutes or less, and administration of the inhalable suspension is achieved over five minutes or less.

In certain embodiments, the present invention provides a method, wherein the time of administration of the composition through the nebulizer and the time of administration of the inhalable suspension are the same.

In certain embodiments, the present invention provides a method, wherein the time of administration of the composition through the nebulizer and the time of administration of the inhalable suspension are different.

In certain embodiments, the present invention provides a method, wherein the composition comprises about 40, about 60, about 120, about 125, about 240, about 250, about 500, about 1000, about 1500, or about 2000 μg of budesonide.

In certain embodiments, the present invention provides a method, wherein the solvent comprises water.

In certain embodiments, the present invention provides a method, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl, β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments, the present invention provides a method, wherein the solubility enhancer comprises SBE7-β-CD.

In certain embodiments, the present invention provides a method, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

In certain embodiments, the present invention provides a method, wherein the bronchoconstrictive disorder is selected from the group consisting of asthma, pediatric asthma, bronchial asthma, allergic asthma, intrinsic asthma, chronic obstructive pulmonary disease (COPD), chronic bronchitis, and emphysema.

### BRIEF DESCRIPTION OF THE FIGURES

FIG. 1 shows percentage of lung deposition and propharyngeal deposition of an inhalable composition comprising budesonide.

FIG. 2 shows total lung deposition of budesonide from scintigraphy data.

FIG. 3 shows percentage of respirable fraction (RF; particle sizes less than 5 μm) determined using different methodologies (laser diffraction 20 L/min, laser diffraction 28.3 L/min, and cascade impaction).

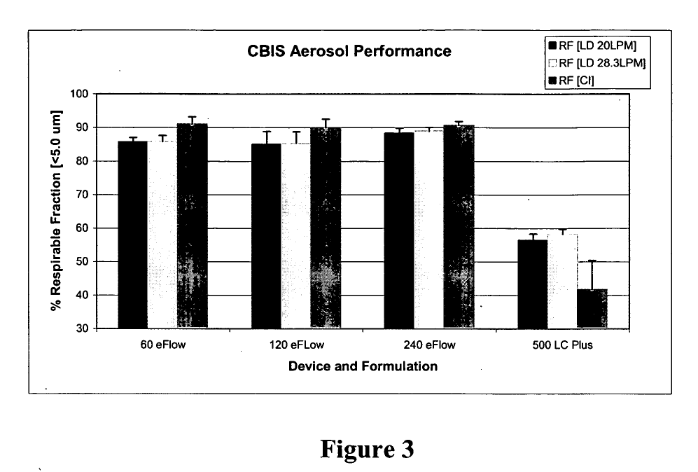

FIG. 4 provides a summary of the mean plasma concentrations following a single dose of Budesonide Administrations A to E. Administration A is 60 μg 99 mTc-DTPA labeled budesonide+SBE7-β-CD inhalation solution delivered with a modified Pari® eFlow nebulizer. Administration B is 120 μg 99 mTc-DTPA labeled budesonide+SBE7-β-CD inhalation solution delivered with a modified Pari® eFlow nebulizer. Administration C is 240 μg 99 mTc-DTPA labeled budesonide+SBE7-β-CD inhalation solution delivered with a modified Pari® eFlow nebulizer. Administration D is 500 μg budesonide suspension (Pulmicort Respules®) delivered with a Pari® LC Plus jet nebulizer. Administration E is 1000 μg budesonide suspension (Pulmicort Respules®) delivered with a Pari® LC Plus jet nebulizer.

FIG. 5 provides a summary of the mean plasma concentrations following twice daily administration of budesonide for seven days. Treatment A (-Δ-) is a 60 μg Captisol-Enabled® Budesonide Inhalation Solution (CBIS) inhalation solution delivered with a Pari® eFlow nebulizer. Treatment B (-◯-) is a 120 μg CBIS inhalation solution delivered with a Pari® eFlow nebulizer. Treatment C (-□-) is a 250 μg budesonide suspension (Pulmicort Respules®) delivered with a Pari® LC Plus jet nebulizer. Treatment D (-⋄-) is a 500 μg budesonide suspension (Pulmicort Respules®) delivered with a Pari® LC Plus jet nebulizer.

FIG. 6 provides a graphical representation of the budesonide content in the LC PLUS nebulizer in μg/ml in Pulmicort Respules (250 μg/2 ml budesonide) and CBIS (120 μg/ml budesonide) formulations after 0, 1, 2, 3 min. nebulization.

FIG. 7 provides a graphical representation of the budesonide content in the LC PLUS nebulizer in % of amount at t=0 in Pulmicort Respules (250 μg/2 ml budesonide) and CBIS (120 μg/ml budesonide) formulations after 0, 1, 2, 3 min. nebulization.

### DETAILED DESCRIPTION OF THE INVENTION

Reference will now be made in detail to embodiments of the inhalable compositions, systems and methods disclosed herein. Examples of the embodiments are illustrated in the following Examples section.

Unless defined otherwise, all technical and scientific terms used herein have the same meaning as is commonly understood by one of skill in the art to which the inventions described herein belong. All patents and publications referred to herein are incorporated by reference.

Certain Definitions

As used herein, the terms “comprising,” “including,” “such as,” and “for example” are used in their open, non-limiting sense.

The term “about” is used synonymously with the term “approximately.” As one of ordinary skill in the art would understand, the exact boundary of “about” will depend on the component of the composition or other parameter. Illustratively, the use of the term “about” with regard to a certain therapeutically effective pharmaceutical dose indicates that values slightly outside the cited values, i.e., plus or minus 0.1% to 10%, which are also effective and safe.

“Administered under the same conditions” or “under the same conditions,” as used herein, refers to two or more methods and/or systems for the delivery of a corticosteroid wherein the methods and/or systems have one or more of the same conditions for administration of the corticosteroid. The conditions for administration can be selected from the group consisting of, but not limited to, the corticosteroid that is administered, the route of delivery of the corticosteroid, the time of administration, the nominal dosage administered to the subject, the number of doses administered, the volume of the dose administered, and the type of nebulizer used for delivery of the corticosteroid, or any combination of the above-recited conditions for administration. In some embodiments, “under the same conditions” can mean that the corticosteroid that is administered is the same. In other embodiments, “under the same conditions” can mean that the route of delivery of the corticosteroid is the same. In yet other embodiments, “under the same conditions” means that the time of administration of the corticosteroid is the same. In still other embodiments, “under the same conditions” means that the nominal dosage of the corticosteroid administered to the subject is the same. In yet still other embodiments, “under the same conditions” means that the number of doses administered is the same. In other embodiments, “under the same conditions” can mean that the time of administration for the corticosteroid is the same, but the nominal dosage of the corticosteroid is different. In still other embodiments, “under the same conditions” can mean that the corticosteroid administered is the same, but the nominal dosage of the corticosteroid is different. In yet still other embodiments, “under the same conditions” can mean that the corticosteroid administered is the same, but the nominal dosage of the corticosteroid and the type of nebulizer used for delivery of the corticosteroid is different. In still yet other embodiments, “under the same conditions” can mean that the nominal dosage of the corticosteroid is the same, but the time of administration for the corticosteroid is different. In other embodiments, “under the same conditions” can mean that the nominal dosage of the corticosteroid is the same, but the type of nebulizer used for delivery of the corticosteroid and the time of delivery are different. In still other embodiments, “under the same conditions” can mean that the corticosteroid administered is the same, but the normal dosage, the type of nebulizer used for delivery of the corticosteroid, and the time of administration are different.

“Bioavailability” refers to the percentage of the weight of a corticosteroid, such as budesonide, dose that is delivered into the general circulation of the animal or human being studied. The total exposure (AUC_{(0-∞)}) of a drug when administered intravenously is usually defined as 100% Bioavailable (F %).

“Blood plasma concentration” refers to the concentration of a corticosteroid, such as budesonide, in the plasma component of blood of a subject or patient population. It is understood that the plasma concentration of a corticosteroid, such as budesonide, may vary significantly between subjects, due to variability with respect to metabolism and/or possible interactions with other therapeutic agents. In accordance with one aspect of the present invention, the blood plasma concentration of a corticosteroid, such as budesonide, may vary from subject to subject. Likewise, values such as maximum plasma concentration (Cₘₐₓ) or time to reach maximum plasma concentration (Tₘₐₓ) or area under the curve from time zero to time of last measurable concentration (AUCₗₐₛₜ) or total area under the plasma concentration time curve (AUC_{(0-∞)}) may vary from subject to subject. Due to this variability, the amount necessary to constitute “a therapeutically effective amount” of a corticosteroid, such as budesonide, may vary from subject to subject.

“Bronchoconstrictive disorders,” as used herein, refers to any disease or condition which can be physically manifested by the constriction or narrowing of the bronchi. Examples of bronchoconstrictive disorders include, but are not limited to, asthma, pediatric asthma, bronchial asthma, allergic asthma, intrinsic asthma, chronic obstructive pulmonary disease (COPD), chronic bronchitis, and emphysema.

“Conventional inhalable corticosteroid therapies” or “inhalable suspensions comprising a corticosteroid,” as used herein, refers to the use of an available suspension-based corticosteroid formulation, for example, the commercially available Pulmicort® Respules (budesonide suspension), in combination with a nebulizer, preferably a jet nebulizer, e.g., Pari LC Jet Plus nebulizer, for the treatment of asthma and/or chronic obstructive pulmonary disease (COPD) or other bronchoconstrictive disorders at therapeutically effective dosages for a given subject, population or populations or those conventional dosages known to those of skill in the art, e.g., for the aforementioned commercial formulation, Pulmicort® Respules, from about 500 μg/day to 2000 μg/day. In particular, a preferred budesonide suspension comparator is Pulmicort® Respules, which are commercially available budesonide suspensions comprising either 250 μg of budesonide suspended in a 2 ml aqueous volume within a unit dose ampoule or 500 μg of budesonide suspended in a 2 ml aqueous volume within in a unit dose ampoule. Additional suspension-based corticosteroid preparations include beclomethasone dipropionate (Clenil®) and fluticasone propionate (Flixotide®), wherein the suspension-based corticosteroid preparations are administered by an inhalation nebulizer at therapeutically effective dosages or those conventional dosages known to those of skill in the art.

“Drug absorption” or “absorption” typically refers to the process of movement of drug from site of delivery of a drug across a barrier into a blood vessel or the site of action, e.g., a drug being absorbed in the pulmonary capillary beds of the alveoli.

“Equal” or “equivalent,” as used herein, refers to two or more parameters or values having substantially the same value. As one of ordinary skill in the art will recognize, the exact boundary of “equal” or “equivalent” will depend on the particular parameter or value being analyzed. Illustratively, the use of the terms “equal” or “equivalent,” as used herein, encompasses values slightly outside the cited values, i.e., plus or minus 0.1% to 25%. For example, in the context of this invention, a Cₘₐₓ of 578.2 (pg/ml) is equal to a Cₘₐₓ of 556.74 pg/ml. Similarly, in another example, a Cₘₐₓ of 1195.3 (pg/ml) is equal to a Cₘₐₓ of 1114.83 pg/ml.

“Inhalation nebulizer,” as used herein, refers to a device that turns medications, compositions, formulations, suspensions, and mixtures, etc. into a fine mist for delivery to the lungs.

“Inhaled aqueous mixture,” “aqueous inhalation mixture,” or “inhalable composition,” as used herein generally refer to any aqueous (including partially aqueous) dosage form for the inhaled delivery of an active agent other than a suspension. Examples of suitable aqueous mixtures or inhalable compositions include, but are not limited to, solutions, dispersions, nanoparticulate dispersions, nanoparticulate suspensions, emulsions, colloidal liquids, micelle or mixed micelle liquids, and liposomal liquids. Other suitable inhaled aqueous mixtures also include suspensions to which solubility enhancers have been added and at least part of the initial suspension has increased solubility.

In some embodiments of the invention, “inhaled aqueous mixture,” “aqueous inhalation mixture,” or “inhalable composition” do not include nano-dispersions and/or nano-suspensions. In other embodiments, “inhaled aqueous mixture,” “aqueous inhalation mixture,” or “inhalable composition” do not include micelle, mixed-micelle liquids or liposomal liquids. In still other embodiments, “inhaled aqueous mixture,” “aqueous inhalation mixture,” or “inhalable composition” do not include nano-dispersions and/or nano-suspensions, micelle, mixed-micelle liquids or liposomal liquids. In other embodiments, “inhalable compositions” include, but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

“Local bioavailability” refers to the fraction of the total dose of a pharmacologic agent that is bioavailable at the site of pharmacologic activity of that agent upon administration of the agent to a patient via a specific delivery route. Local bioavailability is to be contrasted with systemic bioavailability, which is the fraction of the total dose of an administered pharmacologic agent that reaches the systemic circulation of a patient. By way of a non-limiting example, the local bioavailability of an agent delivered by inhalation refers to the fraction of the total dose of the inhaled agent which is delivered to the lungs upon administration via the inhalation route of delivery.

“Nominal dosage,” as used herein, refers to the total amount of the pharmaceutically active agent, e.g., corticosteroid, present in an inhalable dosage form prior to the administration of the inhalable dosage form comprising the pharmaceutically active agent. Thus, by way of a non-limiting example, an aqueous inhalation mixture comprising budesonide at a nominal dosage of 120 μg/dose refers to an aqueous inhalation mixture comprising approximately 120 μg of budesonide prior the administration of the aqueous inhalation mixture to a patient. Likewise, a unit dose ampoule comprising an inhalable suspension with 1000 μg of a corticosteroid, e.g. budesonide, prior to administration, should have a nominal dosage, as used herein, of about 1000 μg/dose if the entire contents of the unit dose ampoule is put into a delivery device, e.g. a nebulize, for administration to a patient.

“Pharmacokinetics” refers to the factors which reflect the attainment and maintenance of a concentration of drug at a site of action.

“Enhanced pharmacokinetic profile,” as used herein, in some embodiments refers to a pharmacokinetic profile wherein one drug formulation (test formulation) displays increased absorption or distribution at the drug's site of action as compared to another drug formulation (reference formulation). In other embodiments, an enhanced pharmacokinetic profile results when administration of an aqueous inhalation mixture provides an equivalent absorption or distribution at the drug's site of action as compared to an inhalable suspension wherein the aqueous inhalation mixture is administered at a lower nominal dosage than the inhalable suspension (e.g., an equivalent absorption of the test formulation at a nominal dosage of 1:2 the reference product equals a two-fold enhanced pharmacokinetic profile, an equivalent absorption of the test formulation at a nominal dosage of 1:3 equals a three-fold pharmacokinetic profile, an equivalent absorption of the test formulation at a nominal dosage of 1:4 equals a four-fold pharmacokinetic profile, etc.). In other embodiments, an enhanced pharmacokinetic profile results when administration of an aqueous inhalation mixture provides greater absorption or distribution at the drug's site of action as compared an inhalable suspension wherein the aqueous inhalation mixture is administered at the same nominal dosage as the inhalable suspension. In certain other embodiments, an enhanced pharmacokinetic profile can be quantified on the basis of the increase in absorption or distribution at the drug's site of action of a aqueous inhalation mixture as compared to a inhalable suspension. For example, in certain embodiments, a two-fold enhanced pharmacokinetic profile results when administration of an aqueous inhalation mixture displays a pharmacokinetic profile wherein the numerical values representing the absorption or distribution at the drug's site of action values of the aqueous inhalation mixture are at least twice (2×) the numerical values representing the absorption or distribution at the drug's site of action values of an inhalable suspension. In some embodiments, the inhalable suspension can be Pulmicort Respules® displaying a pharmacokinetic profile as set forth in the package insert included with the Pulmicort Respules® commercial product (AstraZeneca LP, Wilmington Del., USA).

In some embodiments of this invention, “enhanced lung deposition,” as used herein, refers to lung deposition of a compound wherein one drug formulation (e.g. aqueous inhalation mixture) displays increased total lung deposition as compared to another drug formulation (e.g. inhalable suspension). In some embodiments of this invention, “enhanced lung deposition,” as used herein, refers to lung deposition of a compound wherein one drug formulation (e.g. aqueous inhalation mixture) displays a substantially equivalent total lung deposition as compared to another drug formulation (e.g. inhalable suspension), wherein the aqueous inhalation mixture is administered at a lower nominal dosage than the inhalable suspension. In some embodiments, the inhalable suspension can be Pulmicort® Respules.

As used herein, “respirable fraction” refers to the mass fraction of drug-containing particles exiting the nebulizer or mouthpiece of the nebulizer that is less than about 5 μm in aerodynamic diameter. Respirable fraction relates to the dose of drug leaving the nebulizer, rather than the nominal dose inside the nebulizer.

A “solubility enhancer,” as used herein, includes methods which provide enhanced solubility with or without a chemical agent acting. In certain embodiments, “solubility enhancer” can refer to a chemical agent that increases the solubility of a second chemical compound, such as an active ingredient, in a solvent. In other embodiments, the chemical agent can also be a solvent for the second chemical compound. In still other embodiments, the chemical agent is not a be a solvent for the second chemical compound. In yet other embodiments, “solubility enhancer” can refer to a method of formulation which provides enhanced solubility without a chemical agent acting as the means to increase solubility, e.g., the use of supercritical fluid production methods to generate nanoparticles for dispersion in a solvent.

“Substantially free,” as used herein in some embodiments, refers to a composition or mixture comprising a single therapeutically active agent. In certain embodiments, “substantially free” refers to a composition or mixture comprising a single therapeutically active agent wherein the composition or mixture does not comprise an appreciable amount of a second pharmaceutically active agent, or does not comprise a second pharmaceutically active agent in an amount sufficient to result in therapeutic activity.

A “therapeutically effective amount” or “effective amount” is that amount of a pharmaceutical agent which achieves a pharmacological effect. The term “therapeutically effective amount” includes, for example, a prophylactically effective amount. An “effective amount” of a corticosteroid, such as budesonide, is an amount effective to achieve a desired pharmacologic effect or therapeutic improvement without undue adverse side effects. The effective amount of a corticosteroid, such as budesonide, will be selected by those skilled in the art depending on the particular patient and the disease level. It is understood that “an effect amount” or “a therapeutically effective amount” can vary from subject to subject, and population to population, due to variation in metabolism of a corticosteroid, such as budesonide, age, weight, general condition of the subject, the condition being treated, the severity of the condition being treated, and the judgment of the prescribing physician.

“Treat” or “treatment” as used in the context of a bronchoconstrictive disorder refers to any treatment of a disorder or disease related to the contraction of the bronchi, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treatment” is used synonymously with the terms “prophylaxis” or “prevention.”

I. Inhalable Compositions Comprising a Corticosteroid which Provide Enhanced Lung Deposition

The present invention provides inhalable compositions comprising an effective amount of a corticosteroid, a solvent, and a solubility enhancer which can provide enhanced lung deposition for the delivered corticosteroid as compared to a corticosteroid administered via inhalation in the form of a suspension. In preferred embodiments, the inhalable compositions described herein can enable enhanced lung deposition of the delivered corticosteroid as compared to conventional inhalable corticosteroid therapies and further provide, inter alia, a means for reducing the dosage required to provide a local therapeutic effect. Likewise provided are methods of generating fine particles from an inhalable composition comprise adding a solvent and a solubility enhancer to an effective amount of corticosteroid, and operating a nebulizer to produce fine particles. In other embodiments, the methods provided herein comprise inhalable compositions that enable enhanced lung deposition of the delivered corticosteroid as compared to conventional therapies and further provide, inter alia, a means for reducing the dosage required to provide a local therapeutic effect.

In addition, methods and systems for the treatment of bronchoconstrictive disorders, e.g., asthma and/or chronic obstructive pulmonary disease (COPD), are provided that can enable the delivery of a corticosteroid having enhanced lung deposition as compared to a corticosteroid administered via inhalation in the form of a suspension, wherein the administration by the methods and systems described herein provides one or more of the following advantages: an increase in the lung deposition of the delivered corticosteroid; a method to reduce the nominal dosage of a corticosteroid required to provide a local therapeutic effect; a method to reduce the time required to administer an effective dose of the corticosteroid; a method to increase patient compliance with a therapeutic regimen comprising inhalation of nebulized corticosteroids; a method of enhanced delivery of a corticosteroid; a method for increasing the amount of corticosteroid deposited in the lung, e.g., bronchi and alveoli; and a method for reducing the side effects associated with inhalation of corticosteroids.

Certain aspects of the present invention relate to an inhalable compositions comprising an effective amount of a corticosteroid, a solvent and a solubility enhancer, wherein upon administration of the composition to a subject through a nebulizer, the composition achieves at least about 20% to about 55%, between about 20% to about 50%, or between about 20% to about 40% lung deposition e.g., bronchi and alveoli, based on the amount of corticosteroid in the mixture prior to administration. In certain embodiments, the inhalable compositions comprise an effective amount of a single corticosteroid, a solvent and a solubility enhancer and are substantially free of active pharmaceutical agents other than corticosteroid. In still other embodiments, the composition can achieve at least about 20% to about 55% lung deposition based on the amount of corticosteroid in the mixture prior to administration. In other embodiments, the composition can achieve at least 25% to about 45% lung deposition based on the amount of corticosteroid in the mixture prior to administration. In certain embodiments, the composition achieves at least about 25% lung deposition based on the amount of corticosteroid in the composition prior to administration. In other embodiments, the composition achieves at least about 30% lung deposition based on the amount of corticosteroid in the composition prior to administration. In still other embodiments, the composition achieves at least about 35% lung deposition based on the amount of corticosteroid in the composition prior to administration. In yet still other embodiments, the composition achieves at least about 40% lung deposition based on the amount of corticosteroid in the composition prior to administration. In other embodiments, the composition achieves at least about 45% lung deposition based on the amount of corticosteroid in the composition prior to administration. In still other embodiments, the composition achieves at least about 55% lung deposition based on the amount of corticosteroid in the composition prior to administration. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid and a solubility enhancer and are substantially free of active pharmaceutical agents other than corticosteroid. In still other embodiments, the inhalable compositions comprises an effective amount of a budesonide, a solvent and a solubility enhancer and are substantially free of active pharmaceutical agents other than the budesonide.

In certain embodiments of this invention, the inhalable compositions comprising an effective amount of a corticosteroid, a solvent, and a solubility enhancer which can provide enhanced lung deposition also achieves at least about 60% respirable fraction upon administration. In a more preferred embodiment of this invention, the composition also achieves at least about 70% respirable fraction upon administration. In a still more preferred embodiment of this invention, the composition also achieves at least about 80% respirable fraction upon administration. In the most preferred embodiment of this invention, the composition also achieves at least about 85% respirable fraction upon administration. In certain embodiments, the inhalable compositions comprise an effective amount of a single corticosteroid, a solvent and a solubility enhancer and are substantially free of active pharmaceutical agents other than corticosteroid.

In some embodiments of this invention, the inhalable compositions comprising an effective amount of a corticosteroid, a solvent, and a solubility enhancer which can provide enhanced lung deposition comprise an amount of corticosteroid in the composition prior to administration of about 15 to about 2000 μg of a corticosteroid. In certain embodiments, the inhalable compositions comprise an effective amount of a single corticosteroid, a solvent and a solubility enhancer and are substantially free of active pharmaceutical agents other than corticosteroid. In other embodiments, the inhalable compositions comprising an effective amount of a corticosteroid, a solvent, and a solubility enhancer which can provide enhanced lung deposition comprise an amount of corticosteroid in the composition prior to administration of about 250 to about 2000 μg of a corticosteroid. In still other embodiments, the inhalable compositions comprising an effective amount of a corticosteroid, a solvent, and a solubility enhancer which can provide enhanced lung deposition comprise an amount of corticosteroid in the composition prior to administration of about 60 to about 1500 μg of a corticosteroid. In yet other embodiments, the inhalable compositions comprising an effective amount of a corticosteroid, a solvent, and a solubility enhancer which can provide enhanced lung deposition comprise an amount of corticosteroid in the composition prior to administration of about 100 to about 1000 μg of a corticosteroid. In still other embodiments, the inhalable compositions comprising an effective amount of a corticosteroid, a solvent, and a solubility enhancer which can provide enhanced lung deposition comprise an amount of corticosteroid in the composition prior to administration of about 120 to about 1000 μg of a corticosteroid. In yet still other embodiments, the inhalable compositions comprising an effective amount of a corticosteroid, a solvent, and a solubility enhancer which can provide enhanced lung deposition comprise an amount of corticosteroid in the composition prior to administration of about 125 to about 500 μg of a corticosteroid. In certain embodiments, the inhalable compositions comprising an effective amount of a corticosteroid, a solvent, and a solubility enhancer which can provide enhanced lung deposition comprise an amount of corticosteroid in the composition prior to administration of about 40, about 60, about 100, about 120, about 125, about 240, about 250, about 500, about 1000, about 1500, or about 2000 μg of a corticosteroid. In one embodiment, the inhalable composition comprises an amount of corticosteroid in the composition prior to administration of about 40 μg of a corticosteroid. In another embodiment, the inhalable composition comprises an amount of corticosteroid in the composition prior to administration of about of 60 μg of a corticosteroid. In still another embodiment, the inhalable composition comprises an amount of corticosteroid in the composition prior to administration of about 100 μg of a corticosteroid. In yet another embodiment, the inhalable composition comprises an amount of corticosteroid in the composition prior to administration of about 120 μg of a corticosteroid. In still yet another embodiment, the inhalable composition comprises an amount of corticosteroid in the composition prior to administration of about 125 μg of a corticosteroid. In yet another embodiment, the inhalable composition comprises an amount of corticosteroid in the composition prior to administration of about 240 μg of a corticosteroid. In yet still another embodiment, the inhalable composition comprises an amount of corticosteroid in the composition prior to administration of less than about 250 μg of a corticosteroid. In another embodiment, the inhalable composition comprises an amount of corticosteroid in the composition prior to administration of less than about 500 μg of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, and a solubility enhancer and are substantially free of active pharmaceutical agents other than corticosteroid. In still other embodiments, the inhalable compositions comprises an effective amount of budesonide, and a solubility enhancer and are substantially free of active pharmaceutical agents other than the budesonide.

In certain embodiments, the inhalable compositions can comprise about 40 μg budesonide, a solvent and a solubility enhancer, wherein upon administration of the composition to a subject through a nebulizer, the composition achieves lung deposition of at least 15 μg of budesonide. In certain other embodiments, the inhalable compositions can comprise about 60 μg budesonide, a solvent and a solubility enhancer, wherein upon administration of the composition to a subject through a nebulizer, the composition achieves lung deposition of at least 20 μg of budesonide. In still other embodiments, the inhalable composition can comprise about 120 μg budesonide, a solvent and a solubility enhancer, wherein upon administration of the composition to a subject through a nebulizer, the composition achieves lung deposition of at least 40 μg of budesonide. In yet other embodiments, the inhalable composition can comprise about 240 μg budesonide, a solvent and a solubility enhancer, wherein upon administration of the composition to a subject through a nebulizer, the composition achieves lung deposition of at least 80 μg of budesonide.

In certain embodiments, the inhalable compositions can comprise about 40 μg budesonide, a solvent and a solubility enhancer, wherein upon administration of the composition to a subject through a nebulizer, the composition achieves lung deposition of at least 13 μg of budesonide, wherein the composition is substantially free of active pharmaceutical agents other than the budesonide. In certain other embodiments, the inhalable compositions can comprise about 60 μg budesonide, a solvent and a solubility enhancer, wherein upon administration of the composition to a subject through a nebulizer, the composition achieves lung deposition of at least 20 μg of budesonide, wherein the composition is substantially free of active pharmaceutical agents other than the budesonide. In still other embodiments, the inhalable composition can comprise about 120 μg budesonide, a solvent and a solubility enhancer, wherein upon administration of the composition to a subject through a nebulizer, the composition achieves lung deposition of at least 40 μg of budesonide, wherein the composition is substantially free of active pharmaceutical agents other than the budesonide. In yet other embodiments, the inhalable composition can comprise about 240 μg budesonide, a solvent and a solubility enhancer, wherein upon administration of the composition to a subject through a nebulizer, the composition achieves lung deposition of at least 80 μg of budesonide, wherein the composition is substantially free of active pharmaceutical agents other than the budesonide.

In some embodiments, suitable inhalable compositions comprising a corticosteroid include, but are not limited to, solutions, dispersions, nanoparticulate dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

The corticosteroids that are useful in the inhalable compositions described herein included, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednusone, methylprednisolone, mometasone, paramethasone, prednisolone, prednisone, rofleponide, RPR 106541, tixocortol, triamcinolone, and their respective pharmaceutically acceptable derivatives. In preferred embodiments, the corticosteroid is budesonide. In other preferred embodiments, the corticosteroid is budesonide wherein the budesonide is either an individual diastereomer or a mixture of the two diastereomers administered individually or together for a therapeutic effect.

In some embodiments of the inhalable compositions described herein, the inhalable composition comprises a solvent. In certain embodiments, the solvent is selected from the group comprising water, aqueous alcohol, propylene glycol, or aqueous organic solvent. In preferred embodiments, the solvent is water.

In other embodiments of the inhalable compositions described herein, the inhalable composition comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g., SBE7-β-CD (Captisol®). In one embodiment, the solubility enhancer can have a concentration (w/v) of about 2% when the solubility enhancer is a cyclodextrin or cyclodextrin derivative, e.g., SBE7-β-CD (Captisol®). In another embodiment, the solubility enhancer can have a concentration (w/v) of about 5% when the solubility enhancer is a cyclodextrin or cyclodextrin derivative, e.g., SBE7-β-CD (Captisol®). In yet another embodiment, the solubility enhancer can have a concentration (w/v) about 7% when the solubility enhancer is a cyclodextrin or cyclodextrin derivative, e.g., SBE7-β-CD (Captisol®). In still yet another embodiment, the solubility enhancer can have a concentration (w/v) of about 10% when the solubility enhancer is a cyclodextrin or cyclodextrin derivative, e.g., SBE7-β-CD (Captisol®).

Chemical agents acting as solubility enhancers suitable for use in the present invention include, but are not limited to, propylene glycol, non-ionic surfactants, phospholipids, cyclodextrins and derivatives thereof, and surface modifiers and/or stabilizers. In other embodiments, solubility enhancers refer to a formulation method which provides enhanced solubility without a chemical agent acting as the means to increase solubility, e.g. the use of supercritical fluid production methods to generate nanoparticles for dispersion in a solvent.

Additional solubility enhancers suitable for use in the inhalable compositions described herein are known in the art and are described in, e.g., U.S. Pat. Nos. 5,134,127, 5,145,684, 5,376,645, 6,241,969 and U.S. Pub. Appl. Nos. 2005/0244339 and 2005/0008707, each of which is specifically incorporated by reference herein. In addition, examples of suitable solubility enhancers are described below.

Suitable cyclodextrins and derivatives for use in the present invention are described in the art, for example, Challa et al., AAPS PharmSciTech 6(2): E329-E357 (2005), U.S. Pat. Nos. 5,134,127, 5,376,645, 5,874,418, each of which is specifically incorporated by reference herein. In some embodiments, suitable cyclodextrins or cyclodextrin derivatives for use in the present invention include, but are not limited to, α-cyclodextrins, β-cyclodextrins, γ-cyclodextrins, SAE-CD derivatives (e.g., SBE-α-CD, SBE-β-CD, SBE1-β-CD, SBE4-β-CD, SBE7-β-CD (Captisol®), and SBE-γ-CD) (Cydex, Inc. Lenexa, Kans.), hydroxyethyl, hydroxypropyl (including 2- and 3-hydroxypropyl) and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α-, β- and γ-cyclodextrin; and the maltosyl, glucosyl and maltotriosyl derivatives of α-, β- and γ-cyclodextrin, which may contain one or more sugar residues, e.g. glucosyl or diglucosyl, maltosyl or dimaltosyl, as well as various mixtures thereof, e.g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Procedures for preparing such cyclodextrin derivatives are well-known, for example, from U.S. Pat. No. 5,024,998, and references incorporated by reference therein. Other cyclodextrins suitable for use in the present invention include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. Nos. 6,610,671, 6,479,467, 6,660,804, or 6,509,323, each of which is specifically incorporated by reference herein.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e.g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, UK, 1999) and New Trends in Cyclodextrins and Derivatives (Ed. Dominique Duchene, Editions de Sante, Paris, France, 1991).

Examples of non-ionic surfactants which appear to have a particularly good physiological compatibility for use in the present invention are tyloxapol, polysorbates including, but not limited to, polyoxyethylene (20) sorbitan monolaurate, polyoxyethylene (20) sorbitan monopalmitate, polyoxyethylene (20) sorbitan monostearate (available under the tradename Tweens 20-40-60, etc.), Polysorbate 80, Polyethylene glycol 400; sodium lauryl sulfate; sorbitan laurate, sorbitan palmitate, sorbitan stearate (available under the tradename Span 20-40-60 etc.), benzalkonium chloride, PPO-PEO block copolymers (Pluronics), Cremophor-EL, vitamin E-TPGS (e.g., d-alpha-tocopheryl-polyethyleneglycol-1000-succinate), Solutol-HS-15, oleic acid PEO esters, stearic acid PEO esters, Triton-X100, Nonidet P-40, and macrogol hydroxystearates such as macrogol-15-hydroxystearate.

In some embodiments, the non-ionic surfactants suitable for use in the present invention are formulated with the corticosteroid to form liposome preparations, micelles or mixed micelles. Methods for the preparations and characterization of liposomes and liposome preparations are known in the art. Often, multi-lamellar vesicles will form spontaneously when amphiphilic lipids are hydrated, whereas the formation of small uni-lamellar vesicles usually requires a process involving substantial energy input, such as ultrasonication or high pressure homogenization. Further methods for preparing and characterizing liposomes have been described, for example, by S. Vernuri et al. (Preparation and characterization of liposomes as therapeutic delivery systems: a review. Pharm Acta Helv. 1995, 70(2):95-111) and U.S. Pat. Nos. 5,019,394, 5,192,228, 5,882,679, 6,656,497 each of which is specifically incorporated by reference herein.

In some cases, for example, micelles or mixed micelles may be formed by the surfactants, in which poorly soluble active agents can be solubilized. In general, micelles are understood as substantially spherical structures formed by the spontaneous and dynamic association of amphiphilic molecules, such as surfactants. Mixed micelles are micelles composed of different types of amphiphilic molecules. Both micelles and mixed micelles should not be understood as solid particles, as their structure, properties and behavior are much different from solids. The amphiphilic molecules which form the micelles usually associate temporarily. In a micellar solution, there is a dynamic exchange of molecules between the micelle-forming amphiphile and monomolecularly dispersed amphiphiles which are also present in the solution. The position of the drug molecules which are solubilized in such micelles or mixed micelles depends on the structure of these molecules as well as the surfactants used. For example, it is to be assumed that particularly non-polar molecules are localized mainly inside the colloidal structures, whereas polar substances are more likely to be found on the surface. In one embodiment of a micellar or mixed micellar solution, the average size of the micelles may be less than about 200 nm (as measured by photon correlation spectroscopy), such as from about 10 nm to about 100 nm. Particularly preferred are micelles with average diameters of about 10 nm to about 50 nm. Methods of producing micelles and mixed micelles are known in the art and described in, for example, U.S. Pat. Nos. 5,747,066 and 6,906,042, each of which is specifically incorporated by reference herein.

Phospholipids are defined as amphiphile lipids which contain phosphorus. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols, etc. Lecithins are natural mixtures of various phospholipids which usually have a high proportion of phosphatidyl cholines. Depending on the source of a particular lecithin and its method of extraction and/or enrichment, these mixtures may also comprise significant amounts of sterols, fatty acids, triglycerides and other substances.

Additional phospholipids which are suitable for delivery by inhalation on account of their physiological properties comprise, in particular, phospholipid mixtures which are extracted in the form of lecithin from natural sources such as soja beans (soy beans) or chickens egg yolk, preferably in hydrogenated form and/or freed from lysolecithins, as well as purified, enriched or partially synthetically prepared phospholipids, preferably with saturated fatty acid esters. Of the phospholipid mixtures, lecithin is particularly preferred. The enriched or partially synthetically prepared medium- to long-chain zwitterionic phospholipids are mainly free of unsaturations in the acyl chains and free of lysolecithins and peroxides. Examples for enriched or pure compounds are dimyristoyl phosphatidyl choline (DMPC), distearoyl phosphatidyl choline (DSPC) and dipalmitoyl phosphatidyl choline (DPPC). Of these, DMPC is currently more preferred. Alternatively, phospholipids with oleyl residues and phosphatidyl glycerol without choline residue are suitable for some embodiments and applications of the invention.

In some embodiments, the non-ionic surfactants and phospholipids suitable for use in the present invention are formulated with the corticosteroid to form colloidal structures. Colloidal solutions are defined as mono-phasic systems wherein the colloidal material dispersed within the colloidal solution does not have the measurable physical properties usually associated with a solid material. Methods of producing colloidal dispersions are known in the art, for example as described in U.S. Pat. No. 6,653,319, which is specifically incorporated by reference herein.

Suitable surface modifiers for use in the present invention are described in the art, for example, U.S. Pat. Nos. 5,145,684, 5,510,118, 5,565,188, and 6,264,922, each of which is specifically incorporated by reference herein. Examples of surface modifiers and/or surface stabilizers suitable for use in the present invention include, but are not limited to, hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, e.g., Tween 20™ and Tween 80™ (ICI Specialty Chemicals)), polyethylene glycols (e.g., Carbowaxs 3550™ and 934™ (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68™ and F108™, which are block copolymers of ethylene oxide and propylene oxide), poloxamines (e.g., Tetronic 908™, also known as Poloxamine 908™, which is a tetrafunctional block copolymer derived from sequential addition of propylene oxide and ethylene oxide to ethylenediamine (BASF Wyandotte Corporation, Parsippany, N.J.)), Tetronic 1508™ (T-1508) (BASF Wyandotte Corporation), Tritons X-200™, which is an alkyl aryl polyether sulfonate (Rohm and Haas), Crodestas F-100™, which is a mixture of sucrose stearate and sucrose distearate (Croda Inc.), p-isononylphenoxypoly-(glycidol), also known as Olin-10G™ or Surfactant 10™ (Olin Chemicals, Stamford, Conn.), Crodestas SL-40.RTM. (Croda, Inc.), and SA9OHCO, which is C18H37CH2(—CON(CH3)—CH2(CHOH)4(CH2OH)2 (Eastman Kodak Co.), decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-noyl-β-D-glucopyranoside, octanoyl-N-methylglucamide, n-octyl-β-D-glucopyranoside, octyl β-D-thioglucopyranoside, PEG-phospholipid, PEG-cholesterol, PEG-cholesterol derivative, PEG-vitamin A, PEG-vitamin E, lysozyme, random copolymers of vinyl pyrrolidone and vinyl acetate, and the like. (e.g. hydroxypropyl methylcellulose, hydroxypropylcellulose, polyvinylpyrrolidone, copolymers of vinyl acetate, vinyl pyrrolidone, sodium lauryl sulfate and dioctyl sodium sulfosuccinate).

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15 dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)4 ammonium chloride or bromide, N-alkyl (C12-18) dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkylamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylanmmonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, Mirapol™ and ALKAQUAT™ (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides, imide azolinium salts, protonated quaternary acrylamides, methylated quaternary polymers, such as poly[diallyl dimethylammonium chloride] and poly-[N-methyl vinyl pyridinium chloride], and cationic guar.

In certain embodiments, the inhalable compositions of the present invention comprises a solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain embodiments, the solubility enhancer is SBE7-β-CD (Captisol®).

In addition to aqueous inhalation mixtures or inhalable composition comprising a corticosteroid and a solubility enhancer, it is contemplated herein that in certain embodiments the aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

Furthermore, the processes for producing nanometer sized particles, including SCF, can permit selection of a desired morphology (e.g., amorphous, crystalline, resolved racemic) by appropriate adjustment of the conditions for particle formation during precipitation or condensation. As a consequence of selection of the desired particle form, extended release of the selected medicament can be achieved. These particle fabrication processes are used to obtain nanoparticulates that have high purity, low surface imperfections, low surface charges and low sedimentation rates. Such particle features inhibit particle cohesion, agglomeration and also prevent settling in liquid dispersions. Additionally, because processes such as SCF can separate isomers of certain medicaments, such separation could contribute to the medicament's enhanced activity, effectiveness as well as extreme dose reduction. In some instances, isomer separation also contributes to reduced side effects. In accordance with the present methods and systems, an aqueous inhalation mixture can be a composition fabricated into a powdered form by any process including SCF, spray drying, precipitation and volume exclusion, directly into a collection media, wherein the particulate compound is thus automatically generated into a dispersed formulation. In some embodiments, this formulation can be the final formulation.

In other embodiments of the present invention, the inhalable composition can further comprise a second therapeutic agent selected from the group consisting of a beta2-adrenoreceptor agonist, a dopamine (D2) receptor agonist, an anti-cholinergic agent, and a prophylactic therapeutic. In some embodiments of this invention, second therapeutic agent is a beta2-adrenoreceptor agonist selected from the group comprising albuterol, levalbuterol or pharmaceutical acceptable derivatives thereof.

Beta2-Adrenoreceptor agonists for use in the inhalable compositions provided herein include, but are not limited to, Albuterol (α-1(((1,1-dimethylethyl)amino)methyl)-4-hydroxy-1,3-benzenedimethanol); Bambuterol (dimethylcarbamic acid 5-(2-((1,1-dimethylethyl)amino)-1-hydroxyethyl)-1,3-phenyleneester); Bitolterol (4-methylbenzoic acid 4-(2-((1,1-dimethylethyl) amino)-1-hydroxyethyl)-1,2-phenyleneester); Broxaterol (3-bromo-alpha-(((1,1-dimethylethyl)amino)methyl)-5-isoxazolemethanol); Isoproterenol (4-(1-hydroxy-2-((1-methylethyl-)amino)ethyl)-1,2-benzene-diol); Trimetoquinol (1,2,3,4-tetrahydro-1-((3,4-,5-trimethoxyphenyl)-methyl)-6,7-isoquinolinediol); Clenbuterol (4-amino-3,5-dichloro-alpha-(((1,1-dimethylethyl)amino)methyl)benzenemethanol); Fenoterol (5-(1-hydroxy-2-((2-(4-hydroxyphenyl)-1-methylethyl)amino)ethyl)-1,3-benzenediol); Formoterol (2-hydroxy-5-((1RS)-1-hydroxy-2-(((1RS)-2-(p-methoxyphenyl)-1-methylethyl)amino)ethyl)formanilide); (R,R)-Formoterol; Desformoterol ((R,R) or (S,S)-3-amino-4-hydroxy-alpha-(((2-(4-methoxyphenyl)-1-methyl-ethyl)amino)methyl)benzenemethanol); Hexoprenaline (4,4′-(1,6-hexane-diyl)-bis(imino(1-hydroxy-2,1-ethanediyl)))bis-1,2-benzenediol); Isoetharine (4-(1-hydroxy-2-((1-methylethyl)amino)butyl)-1,2-benzenediol); Isoprenaline (4-(1-hydroxy-2-((1-methylethyl)amino)ethyl)-1,2-benzenediol); Meta-proterenol (5-(1-hydroxy-2-((1-methylethyl)amino)ethyl)-1,3-benzene-diol); Picumeterol (4-amino-3,5-dichloro-α-(((6-(2-(2-pyridinyl)ethoxy)hexyl)-amino)methyl)benzenemethanol); Pirbuterol (α-6-(((1,1-dimethylethyl)-amino)methyl)-3-hydroxy-2,6-pyridinemethanol); Procaterol (((R*,S*)-(+-)-8-hydroxy-5-(1-hydroxy-2-((1-methylethyl) amino)butyl)-2(1H)-quinolin-one); Reproterol ((7-(3-((2-(3,5-dihydroxyphenyl)-2-hydroxyethyl)amino)-propyl)-3,7-dihydro-1,3-dimethyl-1H-purine-2,6-dione); Rimiterol (4-(hydroxy-2-piperidinylmethyl)-1,2-benzenediol); Salbutamol ((+-)-alpha1-(((1,1-dimethylethyl)amino)methyl)-4-hydroxy-1,3-benzenedimethanol); (R)-Salbutamol; Salmeterol ((+-)-4-hydroxy-α-1-(((6-(4-phenylbutoxy)hexyl)-amino)methyl)-1,3-benzenedimethanol); (R)-Salmeterol; Terbutaline (5-(2-((1,1-dimethylethyl)amino)-1-hydroxyethyl)-1,3-benzenediol); Tulobuterol (2-chloro-α-(((1,1-dimethylethyl)amino)methyl)benzenemethanol); and TA-2005 (8-hydroxy-5-((1R)-1-hydroxy-2-(N-((1R)-2-(4-methoxyphenyl)-1-methylethyl)amino)ethyl)carbostyril hydrochloride).

Albuterol sulfate, α1[(tert-Butylamino)methyl]-4-hydroxy-m-xylene-α,α′-diol sulfate (2:1) (salt), is a relatively selective beta2-adrenergic bronchodilator having a chemical formula (C13H21NO3)2.H2SO4. Albuterol inhalation aerosol is indicated for the prevention and relief of bronchospasm in patients 4 years of age and older with reversible obstructive airway disease and for the prevention of exercise-induced bronchospasm in patients 4 years of age and older. Albuterol inhalation solution is indicated for the relief of bronchospasm in patients 2 years of age or older with reversible obstructive airway disease and acute attacks of bronchospasm.

Levalbuterol HCl, (R)-α1-[[(1,1-dimethylethyl)amino]methyl]-4-hydroxy-1,3-benzenedimethanol hydrochloride, having chemistry formula as C13H21NO3.HCl, a relatively selective beta2-adrenergic receptor agonist and is the (R)-enantiomer of the drug albuterol. Xopenex (levalbuterol HCl) Inhalation Solution is supplied in unit-dose vials and requires modulation before by nebulization. Each 3 mL unit-dose vial contains either 0.63 mg of levalbuterol (as 0.73 mg of levalbuterol HCl) or 1.25 mg of levalbuterol (as 1.44 mg of levalbuterol HCl), sodium chloride to adjust tonicity, and sulfuric acid to adjust the pH to 4.0 (3.3 to 4.5). Xopenex (levalbuterol HCl) Inhalation Solution is indicated for the treatment or prevention of bronchospasm in adults and adolescents 12 years of age and older with reversible obstructive airway disease.

Dopamine (D2) receptor agonists include, but are not limited to, Apomorphine ((r)-5,6,6a,7-tetrahydro-6-methyl-4H-dibenzo[de,g]quinoline-10,11-diol); Bromocriptine ((5′.alpha.)-2-bromo-12′-hydroxy-2′-(1-methylethyl)-5′-(2-methylpropyl)ergotaman-3′,6′,18-trione); Cabergoline ((8-β)-N-(3-(dimethylamino)propyl)-N-((ethylamino)carbonyl-1)-6-(2-propenyl)ergoline-8-carboxamide); Lisuride (N′-((8-α)-9,10-dideohydro-6-methylergolin-8-yl)-N,N-diethylurea); Pergolide ((8-β-)-8-((methylthio)methyl)-6-propylergoline); Levodopa (3-hydroxy-L-tryrosine); Pramipexole ((s)-4,5,6,7-tetrahydro-N6-prop-yl-2,6-benzothiazolediamine); Quinpirole hydrochloride (trans-(−)-4aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thia-zolo[4,5-d]azepin-2-amine). Other dopamine D2 receptor agonists for use herein are disclosed in International Patent Application Publication No. WO 99/36095, the relevant disclosure of which is hereby incorporated by reference.

Anti-cholinergic agents for use herein include, but are not limited to, ipratropium bromide, oxitropium bromide, atropine methyl nitrate, atropine sulfate, ipratropium, belladonna extract, scopolamine, scopolamine methobromide, homatropine methobromide, hyoscyamine, isopriopramide, orphenadrine, benzalkonium chloride, tiotropium bromide and glycopyrronium bromide.

Other active ingredients for use in the inhalable compositions described herein include, but are not limited to, IL-5 inhibitors such as those disclosed in U.S. Pat. No. 5,668,110, No. 5,683,983, No. 5,677,280, No. 6,071,910 and No. 5,654,276, each of which is incorporated by reference herein; anti-sense modulators of IL-5 such as those disclosed in U.S. Pat. No. 6,136,603, the relevant disclosure of which is hereby incorporated by reference; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623, which is incorporated by reference herein; tachykinin receptor antagonists such as those disclosed in U.S. Pat. No. 5,691,336, No. 5,877,191, No. 5,929,094, No. 5,750,549 and No. 5,780,467, each of which is incorporated by reference herein; leukotriene receptor antagonists such as montelukast sodium (Singular, R-(E)]-1-[[[1-[3-[2-(7-chloro-2-quinolinyl)-ethenyl]-phenyl]-3-[2-(I-hydroxy-1-methylethyl)-phenyl]-propyl]-thio]-methyl]cyclopropaneacetic acid, monosodium salt), 5-lypoxygenase inhibitors such as zileuton (Zyflo®, Abbott Laboratories, Abbott Park, Ill.), and anti-IgE antibodies such as Xolair (recombinant humanized anti-IgE monoclonal antibody (CGP 51901; IGE 025A; rhuMAb-E25), Genentech, Inc., South San Francisco, Calif.), and topical anesthetics such as lidocaine, N-arylamide, aminoalkylbenzoate, prilocaine, etidocaine (U.S. Pat. No. 5,510,339, U.S. Pat. No. 5,631,267, and U.S. Pat. No. 5,837,713, the relevant disclosures of which are hereby incorporated by reference).

In some embodiments of this invention, the inhalable composition is administered to a patient not more than once a day. In other embodiments, the inhalable composition is administered to a patient not more than twice a day. In some embodiments of this invention, the composition is administered to a patient twice a day or more than twice a day. In still other embodiments, the inhalable composition is administered to a patient not more than once a day in the evening.

Another aspect of this invention relates to an inhalable composition comprising an effective amount of corticosteroid, a solvent and a solubility enhancer, wherein the inhalable composition achieves a higher respirable fraction as compared to an inhalable suspension comprising a corticosteroid administered under the same conditions. In certain embodiments, the inhalable compositions comprising an effective amount of a corticosteroid, a solvent and a solubility enhancer are substantially free of active pharmaceutical agents other than corticosteroids. In a preferred embodiment of this invention, the inhalable composition achieves at least about 10% higher respirable fraction compared to an inhalable suspension comprising the corticosteroid administered under the same conditions. In a more preferred embodiment of this invention, the inhalable composition achieves at least about 15% higher respirable fraction compared to an inhalable suspension comprising the corticosteroid administered under the same conditions. In the most preferred embodiment of this invention, the inhalable composition achieves at least about 20% higher respirable fraction compared to an inhalable suspension comprising the corticosteroid administered under the same conditions.

In certain embodiments, the inhalable compositions comprising an effective amount of a corticosteroid, a solvent and a solubility enhancer described here achieve at least about 5% higher lung deposition compared to an inhalable suspension comprising the corticosteroid administered under the same conditions. In other embodiments, the inhalable composition achieves at least about 10% higher lung deposition compared to an inhalable suspension comprising the corticosteroid administered under the same conditions. In still other embodiments, the inhalable composition achieves at least about 15% higher lung deposition compared to an inhalable suspension comprising the corticosteroid administered under the same conditions. In yet other embodiments, the inhalable composition achieves at least about 20% higher lung deposition compared to an inhalable suspension comprising the corticosteroid administered under the same conditions. In still yet other embodiments, the inhalable composition achieves at least about 25% higher lung deposition compared to an inhalable suspension comprising the corticosteroid administered under the same conditions. In certain embodiments, the inhalable compositions comprising an effective amount of a corticosteroid, a solvent and a solubility enhancer are substantially free of active pharmaceutical agents other than corticosteroids.

In some embodiments of this invention, the inhalable compositions comprising an effective amount of a corticosteroid, a solvent and a solubility enhancer achieves about the same lung deposition of the corticosteroid as compared to an inhalable suspension comprising the corticosteroid, wherein the composition is administered at a lower nominal dosage than the inhalable suspension. In some embodiments of this invention, the inhalable composition achieves about 90% to 110% lung deposition of the corticosteroid as compared to an inhalable suspension comprising the corticosteroid, wherein the composition is administered at a lower nominal dosage than the inhalable suspension. In some embodiments of this invention, the inhalable composition achieves about 80% to 120% lung deposition of the corticosteroid as compared to an inhalable suspension comprising the corticosteroid, wherein the composition is administered at a lower nominal dosage than the inhalable suspension. In some embodiments of this invention, the inhalable composition achieves about 70% to 130% lung deposition of the corticosteroid as compared to an inhalable suspension comprising the corticosteroid, wherein the composition is administered at a lower nominal dosage than the inhalable suspension. In certain embodiments, the inhalable compositions comprising an effective amount of a corticosteroid, a solvent and a solubility enhancer are substantially free of active pharmaceutical agents other than corticosteroids.

In other embodiments of this invention, the inhalable compositions comprising an effective amount of a corticosteroid, a solvent and a solubility enhancer inhalable composition also achieves at least about 10% higher fine particle fraction compared to an inhalable suspension comprising the corticosteroid administered under the same conditions. In a more preferred embodiment of this invention, the inhalable composition achieves also at least about 15% higher fine particle fraction compared to an inhalable suspension comprising the corticosteroid administered under the same conditions. In the most preferred embodiment of this invention, the inhalable composition achieves at least about 20% higher fine particle fraction compared to an inhalable suspension comprising the corticosteroid administered under the same conditions. In certain embodiments, the inhalable compositions comprising an effective amount of a corticosteroid, a solvent and a solubility enhancer are substantially free of active pharmaceutical agents other than corticosteroids.

In certain embodiments, compositions of the present invention can also be administered with a pressurized metered dose inhaler (pMDI). A typical pMDI comprises a propellant, surfactant, and a drug in dissolved or suspended form. The device is designed to be portable and inexpensive as well as protecting the drug from light, oxygen, of moisture, and providing constant metering volume upon administration. Small spray particle size can be achieved after complete propellant evaporation. More volatile propellant used (evaporates faster), smaller particle size can be achieved. The most common technical difficulty is the drug's solubility in propellant. Therefore, solubility enhancers of this invention provide methods and systems for effective administration of corticosteroid, beta2-adrenoreceptor agonist, or their combination using MDI.

In some embodiments of this invention, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain other embodiments, the nebulizer is a Pari eFlow nebulizer.

In still other embodiments, the inhalable composition of the present invention is delivered in a significantly shorter period of time than conventional inhalable corticosteroid therapies. For example, the nebulization time for Pulmicort® Respules administered by a Pari LC Plus jet nebulizer typically takes at least 5 minutes to 8 minutes, and in some cases in excess of 10 minutes. By contrast, the inhalable composition comprising a corticosteroid, such as a budesonide, although not limited to a particular administration time, in preferred embodiments may be administered over a delivery time of less than about 5 minutes to less than about 1.5 minutes. In some embodiments, the delivery time can be about 5 minutes. In other embodiments, the delivery time can be less than about 5 minutes. In certain embodiments, the delivery time can be about 4.5 minutes. In certain other embodiments,. the delivery time can be less than about 4.5 minutes. In still other embodiments, the delivery time can be about 4 minutes. In yet other embodiments, the delivery time can be less than about 4 minutes. In still yet other embodiments, the delivery time can be about 3.5 minutes. In other embodiments, the delivery time can be less than about 3.5 minutes. In yet still other embodiments, the delivery time can be about 3 minutes. In other embodiments, the delivery time can be less than about 3 minutes. In certain embodiments, the delivery time can be about 2.5 minutes. In other certain embodiments, the delivery time can be less than about 2.5 minutes. In still other embodiments, the delivery time can be about 2 minutes. In yet still other embodiments, the delivery time can be less than about 2 minutes. In a preferred embodiment, the delivery time can be about 1.5 minutes. In a more preferred embodiment, the delivery time can be less than about 1.5 minutes.

Another aspect of this invention relates to an inhalable composition comprising albuterol and a solubility enhancer, wherein the composition achieves an enhanced lung deposition as compared to albuterol administered under the same conditions. An aspect of this invention also relates to an inhalable composition comprising albuterol and a solubility enhancer, wherein the composition achieves an enhanced pharmacokinetic profile as compared to albuterol administered under the same conditions. Also an aspect of this invention relates to a method of generating fine particles from an inhalable composition comprising an effective amount of albuterol, operating a Pari eFlow nebulizer, and generating an enhanced lung deposition of albuterol upon administration to a patient. Also another aspect of this invention relates to a method of generating fine particles from an inhalable composition comprising an effective amount of albuterol, operating a Pari eFlow nebulizer, and generating an enhanced pharmacokinetic profile of albuterol upon administration to a patient. In some embodiments of this invention, the inhalable composition comprises a corticosteroid.

II. Inhalable Compositions Comprising a Corticosteroid which Provide a Decreased Increase in the Concentration of the Corticosteroid within a Device

Another aspect of this invention relates to an inhalable composition comprising corticosteroid, a solvent and a solubility enhancer, wherein upon administration of the composition to a subject through a device, the composition achieves a rate of increasing concentration of the corticosteroid inside the device of about 5 μg/ml per minute or less over administration of the corticosteroid through the device. In certain embodiments, the composition achieves a rate of increasing concentration of the corticosteroid inside the device of about 5 μg/ml per minute or less over the first three minutes of administration. In certain other embodiments, the composition achieves a rate of increasing concentration of the corticosteroid inside the device of about 3.5 μg/ml per minute or less over the first three minutes of administration. In certain embodiments, the inhalable compositions comprise about 500 μg or less of a corticosteroid. In certain other embodiments, the inhalable compositions comprise about 250 μg or less of a corticosteroid. In other embodiments, the inhalable compositions comprise about 240 μg or less of a corticosteroid. In yet other embodiments, the inhalable compositions comprise about 120 μg or less of a corticosteroid. In still other embodiments, the inhalable compositions comprise about 60 μg or less of a corticosteroid. In yet still other embodiments, the inhalable compositions comprise about 40 μg or less of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, a solvent and a solubility enhancer and are substantially free of active pharmaceutical agents other than corticosteroid. In still other embodiments, the inhalable compositions comprises an effective amount of a budesonide, a solvent and a solubility enhancer and are substantially free of active pharmaceutical agents other than the budesonide.

In certain other embodiments, the inhalable compositions comprising corticosteroid, a solvent and a solubility enhancer achieve a rate of increase in concentration of the corticosteroid. inside the device of about 5% per minute or less over the first three minutes of administration. In some embodiments of this invention, the corticosteroid is budesonide or a pharmaceutical acceptable derivative. In certain other embodiments, the inhalable compositions comprising an effective amount of a corticosteroid, a solvent and a solubility enhancer are substantially free of active pharmaceutical agents other than corticosteroids.

In certain other embodiments, the inhalable composition comprising an effective amount of a corticosteroid, a solvent and a solubility enhancer, wherein upon administration of the composition to a subject through a device, the composition achieves rate of increasing concentration of the corticosteroid inside the device of about 60% or less or a rate of increasing concentration of the corticosteroid inside the device achieved by an inhalable suspension comprising the corticosteroid without a solubility enhancer administered under the same conditions.

In one embodiment, the rate of increasing concentration of the corticosteroid inside the device is achieved over the first 3 minutes of administration. In another embodiment, the rate of increasing concentration of the corticosteroid inside the device is achieved during the second and third minute of administration. In still another embodiment, the rate of increasing concentration of the corticosteroid inside the device is achieved during the third minute of administration. In certain embodiments, the inhalable compositions comprising an effective amount of a corticosteroid, a solvent and a solubility enhancer are substantially free of active pharmaceutical agents other than corticosteroids. In some embodiments of this invention, the corticosteroid is budesonide or a pharmaceutical acceptable derivative.

In one embodiment, the invention relates to an inhalable composition wherein administration of the composition through the device is achieved over five minutes or less, and administration of the inhalable suspension is achieved over five minutes or less. In another embodiment, the invention relates to an inhalable composition wherein the time of administration of the composition through the device and the time of administration of the inhalable suspension are the same. In still other embodiments, the invention relates to an inhalable composition wherein the time of administration of the composition through the device and the time of administration of the inhalable suspension are different.

In a preferred embodiment of this invention, the composition also achieves at least about 60% respirable fraction upon administration. In a more preferred embodiment of this invention, the composition also achieves at least about 70% respirable fraction upon administration. In a still more preferred embodiment of this invention, the composition also achieves at least about 80% respirable fraction upon administration. In the most preferred embodiment of this invention, the composition also achieves at least about 85% respirable fraction upon administration.

In some embodiments of this invention, the inhalable compositions comprising corticosteroid, a solvent and a solubility enhancer composition which can achieve a rate of increasing concentration of the corticosteroid inside the device of about 5 μg/ml per minute or less over the time of administration comprise about 15 to about 500 μg of a corticosteroid. In other embodiments, the inhalable composition comprises about 50 to about 500 μg of a corticosteroid. In still other embodiments, the inhalable composition comprises about 60 to about 250 μg of a corticosteroid. In yet still other embodiments, the composition comprises about 125 to about 500 μg of a corticosteroid. In certain embodiments, the inhalable composition comprises about 40, 60, 120, 125, 240, 250, 500, 1000, 1500, or 2000 μg of a corticosteroid. In one embodiment, the inhalable composition comprises nominal dosage about of about 40 μg of a corticosteroid. In another embodiment, the inhalable composition comprises nominal dosage about of about 60 μg of a corticosteroid. In yet another embodiment, the inhalable composition comprises nominal dosage about of about 100 μg of a corticosteroid. In still yet another embodiment, the inhalable composition comprises nominal dosage about of about 120 μg of a corticosteroid. In still another embodiment, the inhalable composition comprises nominal dosage about of about 125 μg of a corticosteroid. In still yet another embodiment, the inhalable composition comprises nominal dosage about of about 240 μg of a corticosteroid. In yet still another embodiment, the inhalable composition comprises nominal dosage about of less than about 250 μg of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, a solvent and a solubility enhancer and are substantially free of active pharmaceutical agents other than corticosteroid. In still other embodiments, the inhalable compositions comprises an effective amount of a budesonide, a solvent and a solubility enhancer and are substantially free of active pharmaceutical agents other than the budesonide.

In some embodiments, suitable inhalable compositions comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

The corticosteroids that are useful in the inhalable compositions described herein included, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In preferred embodiments, the corticosteroid is budesonide. In other preferred embodiments, the corticosteroid is budesonide wherein the budesonide is either an individual diastereomer or a mixture of the two diastereomers administered individually or together for a therapeutic effect.

In certain embodiments, the systems and methods described herein comprise a solvent. In certain embodiments, the solvent is selected from the group consisting of water, water/ethanol mixture, aqueous alcohol, propylene glycol, or aqueous organic solvent, or combinations thereof. In certain embodiments, the solvent comprises water. In preferred embodiments, the solvent is water.

In other embodiments of the inhalable compositions described herein, the inhalable composition comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

Chemical agents acting as solubility enhancers suitable for use in the present invention include, but are not limited to, propylene glycol, non-ionic surfactants, phospholipids, cyclodextrins and derivatives thereof, and surface modifiers and/or stabilizers. In other embodiments, solubility enhancers refer to a formulation method which provides enhanced solubility without a chemical agent acting as the means to increase solubility, e.g. the use of super critical fluid production methods to generate nanoparticles for dispersion in a solvent.

Additional solubility enhancers suitable for use in the inhalable compositions described herein are known in the art and are described in, e.g., U.S. Pat. Nos. 5,134,127, 5,145,684, 5,376,645, 6,241,969 and U.S. Pub. Appl. Nos. 2005/0244339 and 2005/0008707, each of which is specifically incorporated by reference herein. In addition, examples of suitable solubility enhancers are described below.

Suitable cyclodextrins and derivatives for use in the present invention are described in the art, for example, Challa et al., AAPS PharmSciTech 6(2): E329-E357 (2005), U.S. Pat. Nos. 5,134,127, 5,376,645, 5,874,418, each of which is specifically incorporated by reference herein. In some embodiments, suitable cyclodextrins or cyclodextrin derivatives for use in the present invention include, but are not limited to, α-cyclodextrins, β-cyclodextrins, γ-cyclodextrins, SAE-CD derivatives (e.g., SBE-α-CD, SBE-β-CD, SBE1-β-CD, SBE4-β-CD, SBE7-β-CD (Captisol®), and SBE-γ-CD) (Cydex, Inc. Lenexa, Kans.), hydroxyethyl, hydroxypropyl (including 2- and 3-hydroxypropyl) and dihydroxypropyl ethers, their corresponding mixed ethers and further mixed ethers with methyl or ethyl groups, such as methylhydroxyethyl, ethyl-hydroxyethyl and ethyl-hydroxypropyl ethers of α-, β- and γ-cyclodextrin; and the maltosyl, glucosyl and maltotriosyl derivatives of α-, β- and γ-cyclodextrin, which may contain one or more sugar residues, e.g. glucosyl or diglucosyl, maltosyl or dimaltosyl, as well as various mixtures thereof, e.g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Procedures for preparing such cyclodextrin derivatives are well-known, for example, from U.S. Pat. No. 5,024,998, and references incorporated by reference therein. Other cyclodextrins suitable for use in the present invention include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. Nos. 6,610,671, 6,479,467, 6,660,804, or 6,509,323, each of which is specifically incorporated by reference herein.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e.g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, UK, 1999) and New Trends in Cyclodextrins and Derivatives (Ed. Dominique Duchene, Editions de Sante, Paris, France, 1991).

Examples of non-ionic surfactants which appear to have a particularly good physiological compatibility for use in the present invention are tyloxapol, polysorbates including, but not limited to, polyoxyethylene (20) sorbitan monolaurate, polyoxyethylene (20) sorbitan monopalmitate, polyoxyethylene (20) sorbitan monostearate (available under the tradename Tweens 20-40-60, etc.), Polysorbate 80, Polyethylene glycol 400; sodium lauryl sulfate; sorbitan laurate, sorbitan palmitate, sorbitan stearate (available under the tradename Span 20-40-60 etc.), benzalkonium chloride, PPO-PEO block copolymers (Pluronics), Cremophor-EL, vitamin E-TPGS (e.g., d-alpha-tocopheryl-polyethyleneglycol-1000-succinate), Solutol-HS-15, oleic acid PEO esters, stearic acid PEO esters, Triton-X100, Nonidet P-40, and macrogol hydroxystearates such as macrogol-15-hydroxystearate.

In some embodiments, the non-ionic surfactants suitable for use in the present invention are formulated with the corticosteroid to form liposome preparations, micelles or mixed micelles. Methods for the preparations and characterization of liposomes and liposome preparations are known in the art. Often, multi-lamellar vesicles will form spontaneously when amphiphilic lipids are hydrated, whereas the formation of small uni-lamellar vesicles usually requires a process involving substantial energy input, such as ultrasonication or high pressure homogenization. Further methods for preparing and characterizing liposomes have been described, for example, by S. Vemuri et al. (Preparation and characterization of liposomes as therapeutic delivery systems: a review. Pharm Acta Helv. 1995, 70(2):95-111) and U.S. Pat. Nos. 5,019,394, 5,192,228, 5,882,679, 6,656,497 each of which is specifically incorporated by reference herein.

In some cases, for example, micelles or mixed micelles may be formed by the surfactants, in which poorly soluble active agents can be solubilized. In general, micelles are understood as substantially spherical structures formed by the spontaneous and dynamic association of amphiphilic molecules, such as surfactants. Mixed micelles are micelles composed of different types of amphiphilic molecules. Both micelles and mixed micelles should not be understood as solid particles, as their structure, properties and behavior are much different from solids. The amphiphilic molecules which form the micelles usually associate temporarily. In a micellar solution, there is a dynamic exchange of molecules between the micelle-forming amphiphile and monomolecularly dispersed amphiphiles which are also present in the solution. The position of the drug molecules which are solubilized in such micelles or mixed micelles depends on the structure of these molecules as well as the surfactants used. For example, it is to be assumed that particularly non-polar molecules are localized mainly inside the colloidal structures, whereas polar substances are more likely to be found on the surface. In one embodiment of a micellar or mixed micellar solution, the average size of the micelles may be less than about 200 nm (as measured by photon correlation spectroscopy), such as from about 10 nm to about 100 nm. Particularly preferred are micelles with average diameters of about 10 to about 50 nm. Methods of producing micelles and mixed micelles are known in the art and described in, for example, U.S. Pat. Nos. 5,747,066 and 6,906,042, each of which is specifically incorporated by reference herein.

Phospholipids are defined as amphiphile lipids which contain phosphorus. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols, etc. Lecithins are natural mixtures of various phospholipids which usually have a high proportion of phosphatidyl cholines. Depending on the source of a particular lecithin and its method of extraction and/or enrichment, these mixtures may also comprise significant amounts of sterols, fatty acids, triglycerides and other substances.

Additional phospholipids which are suitable for delivery by inhalation on account of their physiological properties comprise, in particular, phospholipid mixtures which are extracted in the form of lecithin from natural sources such as soja beans (soy beans) or chickens egg yolk, preferably in hydrogenated form and/or freed from lysolecithins, as well as purified, enriched or partially synthetically prepared phospholipids, preferably with saturated fatty acid esters. Of the phospholipid mixtures, lecithin is particularly preferred. The enriched or partially synthetically prepared medium- to long-chain zwitterionic phospholipids are mainly free of unsaturations in the acyl chains and free of lysolecithins and peroxides. Examples for enriched or pure compounds are dimyristoyl phosphatidyl choline (DMPC), distearoyl phosphatidyl choline (DSPC) and dipalmitoyl phosphatidyl choline (DPPC). Of these, DMPC is currently more preferred. Alternatively, phospholipids with oleyl residues and phosphatidyl glycerol without choline residue are suitable for some embodiments and applications of the invention.

In some embodiments, the non-ionic surfactants and phospholipids suitable for use in the present invention are formulated with the corticosteroid to form colloidal structures. Colloidal solutions are defined as mono-phasic systems wherein the colloidal material dispersed within the colloidal solution does not have the measurable physical properties usually associated with a solid material. Methods of producing colloidal dispersions are known in the art, for example as described in U.S. Pat. No. 6,653,319, which is specifically incorporated by reference herein.

Suitable surface modifiers for use in the present invention are described in the art, for example, U.S. Pat. Nos. 5,145,684, 5,510,118, 5,565,188, and 6,264,922, each of which is specifically incorporated by reference herein. Examples of surface modifiers and/or surface stabilizers suitable for use in the present invention include, but are not limited to, hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, e.g., Tween 20™ and Tween 80™ (ICI Specialty Chemicals)), polyethylene glycols (e.g., Carbowaxs 3550™ and 934™ (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68™ and F108™, which are block copolymers of ethylene oxide and propylene oxide), poloxamines (e.g., Tetronic 908™, also known as Poloxamine 908™, which is a tetrafunctional block copolymer derived from sequential addition of propylene oxide and ethylene oxide to ethylenediamine (BASF Wyandotte Corporation, Parsippany, N.J.)), Tetronic 1508™ (T-1508) (BASF Wyandotte Corporation), Tritons X-200™, which is an alkyl aryl polyether sulfonate (Rohm and Haas), Crodestas F-100™, which is a mixture of sucrose stearate and sucrose distearate (Croda Inc.), p-isononylphenoxypoly-(glycidol), also known as Olin-10G™ or Surfactant 10™ (Olin Chemicals, Stamford, Conn.), Crodestas SL-40.RTM. (Croda, Inc.), and SA9OHCO, which is C18H37CH2(—CON(CH3)—CH2(CHOH)4(CH2OH)2 (Eastman Kodak Co.), decanoyl-N-methylglucamide, n-decyl-β-D-glucopyranoside, n-decyl-β-D-maltopyranoside, n-dodecyl β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-noyl-β-D-glucopyranoside, octanoyl-N-methylglucamide, n-octyl-β-D-glucopyranoside, octyl β-D-thioglucopyranoside, PEG-phospholipid, PEG-cholesterol, PEG-cholesterol derivative, PEG-vitamin A, PEG-vitamin E, lysozyme, random copolymers of vinyl pyrrolidone and vinyl acetate, and the like. (e.g. hydroxypropyl methylcellulose, hydroxypropylcellulose, polyvinylpyrrolidone, copolymers of vinyl acetate, vinyl pyrrolidone, sodium lauryl sulfate and dioctyl sodium sulfosuccinate).

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15 dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)4 ammonium chloride or bromide, N-alkyl (C12-18) dimethylbenzyl ammonium chloride, N-alkyl (C14-18) dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkylamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, Mirapol™ and ALKAQUAT™ (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, arid amine oxides, imide azolinium salts, protonated quaternary acrylamides, methylated quaternary polymers, such as poly[diallyl dimethylammonium chloride] and poly-[N-methyl vinyl pyridinium chloride], and cationic guar.

In certain embodiments, the inhalable compositions of the present invention comprises a solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain other embodiments, the solubility enhancer is SBE7-β-CD (Captisol®).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

Furthermore, the processes for producing nanometer sized particles, including SCF, can permit selection of a desired morphology (e.g., amorphous, crystalline, resolved racemic) by appropriate adjustment of the conditions for particle formation during precipitation or condensation. As a consequence of selection of the desired particle form, extended release of the selected medicament can be achieved. These particle fabrication processes are used to obtain nanoparticulates that have high purity, low surface imperfections, low surface charges and low sedimentation rates. Such particle features inhibit particle cohesion, agglomeration and also prevent settling in liquid dispersions. Additionally, because processes such as SCF can separate isomers of certain medicaments, such separation could contribute to the medicament's enhanced activity, effectiveness as well as extreme dose reduction. In some instances, isomer separation also contributes to reduced side effects. In accordance with the present methods and systems, an aqueous inhalation mixture can be a composition fabricated into a powdered form by any process including SCF, spray drying, precipitation and volume exclusion, directly into a collection media, wherein the particulate compound is thus automatically generated into a dispersed formulation. In some embodiments, this formulation can be the final formulation.

In some embodiments of this invention, the inhalable composition further comprises a second therapeutic agent selected from the group consisting of a beta2-adrenoreceptor agonist, a prophylactic therapeutic, and an anti-cholinergic agent. In some embodiments of this invention, the beta2-adrenoreceptor agonist is albuterol, levalbuterol or a pharmaceutical acceptable derivative.

In some embodiments of this invention, the inhalable composition is administered to a patient not more than once a day. In other embodiments, the inhalable composition is administered to a patient not more than twice a day. In some embodiments of this invention, the composition is administered to a patient twice a day or more than twice a day. In still other embodiments, the inhalable composition is administered to a patient not more than once a day in the evening.

In some embodiments of this invention, the device is a nebulizer. In certain embodiments, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In certain other embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain other embodiments, the nebulizer is a Pari eFlow nebulizer.

III. Methods for Generating Fine Particles from an Inhalable Composition Comprising Corticosteroids which Provides Enhanced Lung Deposition

The present invention further provides a method of generating fine particles from an inhalable composition comprising adding a solvent and a solubility enhancer to an effective amount of corticosteroid, and operating a nebulizer to produce fine particles of said composition, wherein upon administration of the composition to a subject through the nebulizer, the method achieves at least about 20% to about 40%, between about 20% to about 50%, or between about 20% to about 55% lung deposition e.g., bronchi and alveoli, based on the amount of corticosteroid in the mixture prior to administration. In certain embodiments, the inhalable compositions comprising an effective amount of a single corticosteroid, a solvent and a solubility enhancer and are substantially free of active pharmaceutical agents other than corticosteroids. In some embodiments, the methods can achieve at least about 25% to about 45% lung deposition based on the amount of corticosteroid in the mixture prior to administration. In other embodiments, the methods can achieve at least 35% to about 40% lung deposition based on the amount of corticosteroid in the mixture prior to administration. In certain embodiments, the composition achieves at least about 25% lung deposition based on the amount of corticosteroid in the composition prior to administration. In other embodiments, the composition achieves at least about 30% lung deposition based on the amount of corticosteroid in the composition prior to administration. In still other embodiments, the composition achieves at least about 35% lung deposition based on the amount of corticosteroid in the composition prior to administration. In yet still other embodiments, the composition achieves at least about 40% lung deposition based on the amount of corticosteroid in the composition prior to administration. In other embodiments, the composition achieves at least about 45% lung deposition based on the amount of corticosteroid in the composition prior to administration. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid and a solubility enhancer and are substantially free of active pharmaceutical agents other than corticosteroid. In still other embodiments, the inhalable compositions comprises an effective amount of a budesonide, and a solubility enhancer and are substantially free of active pharmaceutical agents other than the budesonide.

In a preferred embodiment, the methods of generating fine particles from an inhalable composition also achieve at least about 60% respirable fraction upon administration. In a more preferred embodiment of this invention, the methods also achieve at least about 70% respirable fraction upon administration. In a still more preferred embodiment of this invention, the methods also achieve at least about 80% respirable fraction upon administration. In the most preferred embodiment of this invention, the methods also achieve at least about 85% respirable fraction upon administration.

In some embodiments of this invention, the methods of generating fine particles from an inhalable composition comprising a corticosteroid comprise an amount of corticosteroid in the composition prior to administration of about 15 to about 2000 μg of a corticosteroid. In other embodiments, the inhalable compositions comprising an effective amount of a corticosteroid, a solvent, and a solubility enhancer which can provide enhanced lung deposition comprise an amount of corticosteroid in the composition prior to administration of about 250 to about 2000 μg of a corticosteroid. In still other embodiments, the inhalable compositions comprising an effective amount of a corticosteroid, a solvent, and a solubility enhancer which can provide enhanced lung deposition comprise an amount of corticosteroid in the composition prior to administration of about 60 to about 1500 μg of a corticosteroid. In yet other embodiments, the inhalable compositions comprising an effective amount of a corticosteroid, a solvent, and a solubility enhancer which can provide enhanced lung deposition comprise an amount of corticosteroid in the composition prior to administration of about 100 to about 1000 μg of a corticosteroid. In still other embodiments, the inhalable compositions comprising an effective amount of a corticosteroid, a solvent, and a solubility enhancer which can provide enhanced lung deposition comprise an amount of corticosteroid in the composition prior to administration of about 120 to about 1000 μg of a corticosteroid. In yet still other embodiments, the inhalable compositions comprising an effective amount of a corticosteroid, a solvent, and a solubility enhancer which can provide enhanced lung deposition comprise an amount of corticosteroid in the composition prior to administration of about 125 to about 500 μg of a corticosteroid. In certain embodiments, the inhalable compositions comprising an effective amount of a corticosteroid, a solvent, and a solubility enhancer which can provide enhanced lung deposition comprise an amount of corticosteroid in the composition prior to administration of about 40, about 60, about 100, about 120, about 125, about 240, about 250, about 500, about 1000, about 1500, or about 2000 μg of a corticosteroid. In one embodiment, the inhalable composition comprises an amount of corticosteroid in the composition prior to administration of about 40 μg of a corticosteroid. In another embodiment, the inhalable composition comprises an amount of corticosteroid in the composition prior to administration of about of 60 μg of a corticosteroid. In still another embodiment, the inhalable composition comprises an amount of corticosteroid in the composition prior to administration of about 100 μg of a corticosteroid. In yet another embodiment, the inhalable composition comprises an amount of corticosteroid in the composition prior to administration of about 120 μg of a corticosteroid. In still yet another embodiment, the inhalable composition comprises an amount of corticosteroid in the composition prior to administration of about 125 μg of a corticosteroid. In yet another embodiment, the inhalable composition comprises an amount of corticosteroid in the composition prior to administration of about 240 μg of a corticosteroid. In yet still another embodiment, the inhalable composition comprises an amount of corticosteroid in the composition prior to administration of less than about 250 μg of a corticosteroid. In another embodiment, the inhalable composition comprises an amount of corticosteroid in the composition prior to administration of less than about 500 μg of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, a solvent and a solubility enhancer and are substantially free of active pharmaceutical agents other than corticosteroid. In still other embodiments, the inhalable compositions comprises an effective amount of a budesonide, a solvent and a solubility enhancer and are substantially free of active pharmaceutical agents other than the budesonide.

In some embodiments, suitable inhalable compositions comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, the inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

The corticosteroids that are useful in the inhalable compositions described herein included, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In preferred embodiments, the corticosteroid is budesonide. In other preferred embodiments, the corticosteroid is budesonide wherein the budesonide is either an individual diastereomer or a mixture of the two diastereomers administered individually or together for a therapeutic effect.

In some embodiments of the inhalable compositions described herein, the inhalable composition comprises a solvent. In certain embodiments, the solvent is selected from the group comprising water, aqueous alcohol, propylene glycol, or aqueous organic solvent. In preferred embodiments, the solvent is water.

In other embodiments of the inhalable compositions described herein, the inhalable composition comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

In other embodiments, the inhalable composition for use in the present methods further comprises a solubility enhancer. In certain embodiments, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In preferred embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain other embodiments, the solubility enhancer is SBE7-β-CD (Captisol®).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein.

In some embodiments of this invention, the inhalable composition for use in the present methods further comprises a second therapeutic agent selected from the group consisting of a beta2-adrenoreceptor agonist, a prophylactic therapeutic, and an anti-cholinergic agent. In other embodiments of this invention, the beta2-adrenoreceptor agonist is albuterol, levalbuterol or a pharmaceutical acceptable derivative.

In some embodiments of this invention, the present methods comprise administering the inhalable composition described herein to a patient no more than once a day. In other embodiments of this invention, present methods comprise administering the inhalable composition described herein to a patient twice a day or more than twice a day.

Any known inhalation nebulizer is suitable for use in the presently described invention. Such nebulizers include, e.g., jet nebulizers, ultrasonic nebulizers, pulsating membrane nebulizers, nebulizers with a vibrating mesh or plate with multiple apertures, and nebulizers comprising a vibration generator and an aqueous chamber (e.g., Pari eFlow®). Commercially available air driven jet, ultrasonic or pulsating membrane nebulizers suitable for use in the present invention include the Aeroneb®, Aeroneb GO® (Aerogen, San Francisco, Calif.), Pari LC PLUS®, Pari Boy® N and Pari Duraneb® (PARI Respiratory Equipment, Inc., Monterey, Calif.), MicroAir® (Omron Healthcare, Inc., Vernon Hills, Ill.), Halolite® (Profile Therapeutics Inc., Boston, Mass.), Respimat® (Boehringer Ingelheim Ingelheim, Germany), Aerodose® (Aerogen, Inc., Mountain View, Calif.), Omron Elite® (Omron Healthcare, Inc., Vernon Hills, Ill.), Omron Microair® (Omron Healthcare, Inc., Vernon Hills, Ill.), Mabismist II® (Mabis Healthcare, Inc., Lake Forest, Ill.), Lumiscope® 6610, (The Lumiscope Company, Inc., East Brunswick, N.J.), Airsep Mystique®, (AirSep Corporation, Buffalo, N.Y.), Acorn-1 and Acorn-II (Vital Signs, Inc., Totowa, N.J.), Aquatower® (Medical Industries America, Adel, Iowa), Ava-Neb® (Hudson Respiratory Care Incorporated, Temecula, Calif.), Cirrus® (Intersurgical Incorporated, Liverpool, N.Y.), Dart® (Professional Medical Products, Greenwood, S.C.), Devilbiss® Pulmo Aide (DeVilbiss Corp. Somerset, Pa.), Downdraft® (Marquest, Englewood, Colo.), Fan Jet® (Marquest, Englewood, Colo.), MB-5 (Mefar, Bovezzo, Italy), Misty Neb® (Baxter, Valencia, Calif.), Salter 8900 (Salter Labs, Arvin, Calif.), Sidestream® (Medic-Aid, Sussex, UK), Updraft-II® (Hudson Respiratory Care; Temecula, Calif.), Whisper Jet® (Marquest Medical Products, Englewood, Colo.), Aiolos® (Aiolos Medicnnsk Teknik, Karlstad, Sweden), Inspiron® (Intertech Resources, Inc., Bannockburn, Ill.), Optimist® (Unomedical Inc., McAllen, Texas), Prodomo®, Spira® (Respiratory Care Center, Hameenlinna, Finland), AERx (Aradigm Corporation, Hayward, Calif.), Sonik® LDI Nebulizer (Evit Labs, Sacramento, Calif.), and Swirler W Radioaerosol System (AMICI, Inc., Spring City, Pa.).

Any of these and other known nebulizers can be used to deliver the aqueous inhalation mixtures described in the present invention. In some embodiments, the nebulizers are available from, e.g., Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others.

Other nebulizers suitable for use in the methods and systems describe herein include, but are not limited to, jet nebulizers (optionally sold with compressors), ultrasonic nebulizers, and others. Exemplary jet nebulizers for use herein include Pari LC plus/ProNeb, Pari LC plus/ProNeb Turbo, Pari LCPlus/Dura Neb 1000 & 2000 Pari LC plus/Walkhaler, Pari LC plus/Pari Master, Pari LC star, Omron CompAir XL Portable Nebulizer System (NE-C18 and JetAir Disposable nebulizer), Omron compare Elite Compressor Nebulizer System (NE-C21 and Elite Air Reusable Nebulizer, Pari LC Plus or Pari LC Star nebulizer with Proneb Ultra compressor, Pulomo-aide, Pulmo-aide LT, Pulmo-aide traveler, Invacare Passport, Inspiration Healthdyne 626, Pulmo-Neb Traveler, DeVilbiss 646, Whisper Jet, AcornII, Misty-Neb, Allied aerosol, Schuco Home Care, Lexan Plasic Pocet Neb, Side Stream Hand Held Neb, Mobil Mist, Up-Draft, Up-DraftII, T Up-Draft, ISO-NEB, Ava-Neb, Micro Mist, and PuImoMate.

Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer, and Mabismist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb Portable Nebulizer System, Aerodose Inhaler, and AeroEclipse Breath Actuated Nebulizer. Exemplary nebulizers comprising a vibrating mesh or plate with multiple apertures are described by R. Dhand in New Nebuliser Technology—Aerosol Generation by Using a Vibrating Mesh or Plate with Multiple Apertures, Long-Term Healthcare Strategies 2003, (July 2003), p. 1-4 and Respiratory Care, 47: 1406-1416 (2002), the entire disclosure of each of which is hereby incorporated by reference.

Additional nebulizers suitable for use in the presently described invention include nebulizers comprising a vibration generator and an aqueous chamber. Such nebulizers are sold commercially as, e.g., Pari eFlow, and are described in U.S. Pat. Nos. 6,962,151, 5,518,179, 5,261,601, and 5,152,456, each of which is specifically incorporated by reference herein.

The parameters used in nebulization, such as flow rate, mesh membrane size, aerosol inhalation chamber size, mask size and materials, valves, and power source may be varied in accordance with the principles of the present invention to maximize their use with different types and aqueous inhalation mixtures or different types of corticosteroids.

In addition to the above cited nebulizers, atomizers are also suitable for the systems and methods described herein for the delivery of an aqueous inhalation solution comprising a corticosteroid and a solubility enhancer. Atomizers are known in the art and are described in, for example, U.S. Pat. Nos. 5,954,047, 6,026,808, 6,095,141 and 6,527,151, each of which is specifically incorporated by reference.

In certain embodiments of this invention, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain other embodiments, the nebulizer is a Pari eFlow nebulizer.

IV. Methods for Generating Fine Particles from an Inhalable Compositions Comprising a Corticosteroid which Provide a Decreased Increase in the Concentration of the Corticosteroid within a Device

Another aspect of this invention relates to a method of generating fine particles from an inhalable composition comprising forming the composition by adding a solvent and a solubility enhancer to an effective amount of corticosteroid, and operating a nebulizer to produce fine particles of said composition, wherein upon administration of the composition to a subject through the nebulizer, the composition achieves rate of increasing concentration of the corticosteroid inside the device of about 60% or less of a rate of increasing concentration of the corticosteroid inside the device achieved by an inhalable suspension comprising the corticosteroid without a solubility enhancer administered under the same conditions. In certain embodiments, the methods comprise an inhalable composition comprising a single corticosteroid that is substantially free of active pharmaceutical agents other than the corticosteroid.

In certain embodiments, the rate of increasing concentration of the corticosteroid inside the device is achieved over the first 3 minutes of administration. In other embodiments, the rate of increasing concentration of the corticosteroid inside the device is achieved during the second and third minute of administration. In still other embodiments, the rate of increasing concentration of the corticosteroid inside the device is achieved during the third minute of administration.

In one embodiment, the invention relates to an inhalable composition wherein administration of the composition through the device is achieved over five minutes or less, and administration of the inhalable suspension is achieved over five minutes or less. In another embodiment, the invention relates to an inhalable composition wherein the time of administration of the composition through the device and the time of administration of the inhalable suspension are the same. In still other embodiments, the invention relates to an inhalable composition wherein the time of administration of the composition through the device and the time of administration of the inhalable suspension are different.

In certain embodiments of this invention, the inhalable composition also achieves at least about 60% respirable fraction upon administration. In a more preferred embodiment of this invention, the inhalable composition also achieves at least about 70% respirable fraction upon administration. In a still more preferred embodiment of this invention, the inhalable composition also achieves at least about 80% respirable fraction upon administration. In the most preferred embodiment of this invention, the inhalable composition also achieves at least about 85% respirable fraction upon administration

In some embodiments of this invention, the inhalable composition comprises about 15 to about 2000 μg of a corticosteroid. In other embodiments, the composition comprises about 50 to about 2000 μg of a corticosteroid. In still other embodiments, the composition comprises about 60 to about 1500 μg of a corticosteroid. In yet other embodiments, the composition comprises about 120 to about 1000 μg of a corticosteroid. In yet still other embodiments, the composition comprises about 125 to about 500 μg of a corticosteroid. In some embodiments of this invention, the composition comprises about 40, 60, 120, 125, 240, 250, 500, 1000, 1500, or 2000 μg of said corticosteroid. In some embodiments of this invention, the composition comprises a nominal dosage of from about 60 to 2000 μg of said corticosteroid. In one embodiment, the inhalable composition comprises a nominal dosage of about 40 μg of a corticosteroid. In another embodiment, the inhalable composition comprises a nominal dosage of about 60 μg of a corticosteroid. In yet another embodiment, the inhalable composition comprises a nominal dosage of about 100 μg of a corticosteroid. In yet another embodiment, the inhalable composition comprises a nominal dosage of about 120 μg of a corticosteroid. In still another embodiment , the inhalable composition comprises a nominal dosage of about 125 μg of a corticosteroid. In yet still another embodiment, the inhalable composition comprises a nominal dosage of about 240 μg of a corticosteroid. In still yet another embodiment, the inhalable composition comprises a nominal dosage of less than about 250 μg of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, a solvent and a solubility enhancer and are substantially free of active pharmaceutical agents other than corticosteroid. In still other embodiments, the inhalable compositions comprises an effective amount of a budesonide, a solvent and a solubility enhancer and are substantially free of active pharmaceutical agents other than the budesonide.

In some embodiments, suitable inhalable compositions comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid; such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

The corticosteroids that are useful in the inhalable compositions described herein included, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In preferred embodiments, the corticosteroid is budesonide. In other preferred embodiments, the corticosteroid is budesonide wherein the budesonide is either an individual diastereomer or a mixture of the two diastereomers administered individually or together for a therapeutic effect.

In some embodiments of the inhalable compositions described herein, the inhalable composition comprises a solvent. In certain embodiments, the solvent is selected from the group comprising water, aqueous alcohol, propylene glycol, or aqueous organic solvent. In preferred embodiments, the solvent is water.

In other embodiments of the inhalable compositions described herein, the inhalable composition comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

In other embodiments, the inhalable composition for use in the present methods further comprises a solubility enhancer. In certain embodiments, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In preferred embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain other embodiments, the solubility enhancer is SBE7-β-CD (Captisol®).

In addition to aqueous inhalation mixtures or inhalable composition comprising a corticosteroid and a solubility enhancer; it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

In some embodiments of this invention, the inhalable composition for use in the present methods further comprises a second therapeutic agent selected from the group consisting of a beta2-adrenoreceptor agonist, a prophylactic therapeutic, and an anti-cholinergic agent. In other embodiments of this invention, the beta2-adrenoreceptor agonist is albuterol, levalbuterol or a pharmaceutical acceptable derivative.

In some embodiments of this invention, the inhalable composition is administered to a patient not more than once a day. In other embodiments, the inhalable composition is administered to a patient not more than twice a day. In some embodiments of this invention, the composition is administered to a patient twice a day or more than twice a day. In still other embodiments, the inhalable composition is administered to a patient not more than once a day in the evening.

In certain embodiments of this invention, the device is a nebulizer. In certain other embodiments, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain other embodiments, the nebulizer is a Pari eFlow nebulizer.

Any known inhalation nebulizer is suitable for use in the presently described invention. Such nebulizers include, e.g., jet nebulizers, ultrasonic nebulizers, pulsating membrane nebulizers, nebulizers with a vibrating mesh or plate with multiple apertures, and nebulizers comprising a vibration generator and an aqueous chamber (e.g., Pari eFlow®). Commercially available air driven jet, ultrasonic or pulsating membrane nebulizers suitable for use in the present invention include the Aeroneb®, Aeroneb GO® (Aerogen, San Francisco, Calif.), Pari LC PLUS®, Pari Boy® N and Pari Duraneb®) (PARI Respiratory Equipment, Inc., Monterey, Calif.), MicroAir® (Omron Healthcare, Inc., Vernon Hills, Ill.), Halolite® (Profile Therapeutics Inc., Boston, Mass.), Respimat® (Boehringer Ingelheim Ingelheim, Germany), Aerodose® (Aerogen, Inc., Mountain View, Calif.), Omron Elite® (Omron Healthcare, Inc., Vernon Hills, Ill.), Omron Microair® (Omron Healthcare, Inc., Vernon Hills, Ill.), Mabismist II® (Mabis Healthcare, Inc., Lake Forest, Ill.), Lumiscope® 6610, (The Lumiscope Company, Inc., East Brunswick, N.J.), Airsep Mystique®, (AirSep Corporation, Buffalo, N.Y.), Acorn-1 and Acorn-II (Vital Signs, Inc., Totowa, N.J.), Aquatower® (Medical Industries America, Adel, Iowa), Ava-Neb® (Hudson Respiratory Care Incorporated, Temecula, Calif.), Cirrus® (Intersurgical Incorporated, Liverpool, N.Y.), Dart® (Professional Medical Products, Greenwood, S.C.), Devilbiss® Pulmo Aide (DeVilbiss Corp. Somerset, Pa.), Downdraft® (Marquest, Englewood, Colo.), Fan Jet® (Marquest, Englewood, Colo.), MB-5 (Mefar, Bovezzo, Italy), Misty Neb® (Baxter, Valencia, Calif.), Salter 8900 (Salter Labs, Arvin, Calif.), Sidestream® (Medic-Aid, Sussex, UK), Updraft-II® (Hudson Respiratory Care; Temecula, Calif.), Whisper Jet® (Marquest Medical Products, Englewood, Colo.), Aiolos® (Aiolos Medicnnsk Teknik, Karlstad, Sweden), Inspiron® (Intertech Resources, Inc., Bannockburn, Ill.), Optimist® (Unomedical Inc. , McAllen, Texas), Prodomo®, Spira® (Respiratory Care Center, Hameenlinna, Finland), AERx (Aradigm Corporation, Hayward, Calif.), Sonik® LDI Nebulizer (Evit Labs, Sacramento, Calif.), and Swirler W Radioaerosol System (AMICI, Inc., Spring City, Pa.).

Any of these and other known nebulizers can be used to deliver the aqueous inhalation mixtures described in the present invention. In some embodiments, the nebulizers are available from, e.g., Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others.

Other nebulizers suitable for use in the methods and systems describe herein include, but are not limited to, jet nebulizers (optionally sold with compressors), ultrasonic nebulizers, and others. Exemplary jet nebulizers for use herein include Pari LC plus/ProNeb, Pari LC plus/ProNeb Turbo, Pari LCPlus/Dura Neb 1000 & 2000 Pari LC plus/Walkhaler, Pari LC plus/Pari Master, Pari LC star, Omron CompAir XL Portable Nebulizer System (NE-C18 and JetAir Disposable nebulizer), Omron compare Elite Compressor Nebulizer System (NE-C21 and Elite Air Reusable Nebulizer, Pari LC Plus or Pari LC Star nebulizer with Proneb Ultra compressor, Pulomo-aide, Pulmo-aide LT, Pulmo-aide traveler, Invacare Passport, Inspiration Healthdyne 626, Pulmo-Neb Traveler, DeVilbiss 646, Whisper Jet, AcornII, Misty-Neb, Allied aerosol, Schuco Home Care, Lexan Plasic Pocet Neb, Side Stream Hand Held Neb, Mobil Mist, Up-Draft, Up-DraftII, T Up-Draft, ISO-NEB, Ava-Neb, Micro Mist, and PulmoMate.

Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer, and Mabismist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb Portable Nebulizer System, Aerodose Inhaler, and AeroEclipse Breath Actuated Nebulizer. Exemplary nebulizers comprising a vibrating mesh or plate with multiple apertures are described by R. Dhand in New Nebuliser Technology—Aerosol Generation by Using a Vibrating Mesh or Plate with Multiple Apertures, Long-Term Healthcare Strategies 2003, (July 2003), p. 1-4 and Respiratory Care, 47: 1406-1416 (2002), the entire disclosure of each of which is hereby incorporated by reference.

Additional nebulizers suitable for use in the presently described invention include nebulizers comprising a vibration generator and an aqueous chamber. Such nebulizers are sold commercially as, e.g., Pari eFlow, and are described in U.S. Pat. Nos. 6,962,151, 5,518,179, 5,261,601, and 5,152,456, each of which is specifically incorporated by reference herein.

The parameters used in nebulization, such as flow rate, mesh membrane size, aerosol inhalation chamber size, mask size and materials, valves, and power source may be varied in accordance with the principles of the present invention to maximize their use with different types and aqueous inhalation mixtures or different types of corticosteroids.

In addition to the above cited nebulizers, atomizers are also suitable for the systems and methods described herein for the delivery of an aqueous inhalation solution comprising a corticosteroid and a solubility enhancer. Atomizers are known in the art and are described in, for example, U.S. Pat. Nos. 5,954,047, 6,026,808, 6,095,141 and 6,527,151, each of which is specifically incorporated by reference.

VI. Corticosteroid Inhalation System for Achieving Enhanced Lung Deposition

Another aspect of this invention relates to a system for delivering a therapeutically effective dose of a corticosteroid to a patient comprising (a) an aqueous inhalation mixture comprising the corticosteroid and a solubility enhancer, and (b) a nebulizer, whereby upon administration of the composition to a subject through a nebulizer, the system achieves at least about 20% to about 40%, between about 20% to about 50%, or between about 20% to about 55% lung deposition e.g., bronchi and alveoli, based on the amount of corticosteroid in the mixture prior to administration. In some embodiments, the system can achieve at least about 25% to about 45% lung deposition based on the amount of corticosteroid in the mixture prior to administration. In other embodiments, the system can achieve at least 35% to about 40% lung deposition based on the amount of corticosteroid in the mixture prior to administration. In certain embodiments, the system achieves about 25% lung deposition based on the amount of corticosteroid in the mixture prior to administration. In other embodiments, the system achieves at least about 30% lung deposition based on the amount of corticosteroid in the mixture prior to administration. In still other embodiments, the system achieves at least about 35% lung deposition based on the amount of corticosteroid in the mixture prior to administration. In yet other embodiments, the system achieves at least about 40% lung deposition based on the amount of corticosteroid in the mixture prior to administration. In still other embodiments, the system achieves at least about 45% lung deposition based on the amount of corticosteroid in the mixture prior to administration. In yet still other embodiments, the system achieves at least about 50% lung deposition based on the amount of corticosteroid in the mixture prior to administration. In still another embodiment, the system achieves at least about 40% to about 55% lung deposition based on the amount of corticosteroid in the mixture prior to administration. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, and a solubility enhancer and are substantially free of active pharmaceutical agents other than corticosteroid. In still other embodiments, the inhalable compositions comprises an effective amount of a budesonide, and a solubility enhancer and are substantially free of active pharmaceutical agents other than the budesonide.

In certain embodiments of this invention, the system for delivering delivering a therapeutically effective dose of an aqueous inhalation mixture comprising the corticosteroid by said nebulizer produces at least about 60% respirable fraction. In other embodiments, the system for delivering a therapeutically effective dose of an aqueous inhalation mixture comprising the corticosteroid by said nebulizer produces at least about 75% respirable fraction. In still other embodiments, the system for delivering a therapeutically effective dose of an aqueous inhalation mixture comprising the corticosteroid by said nebulizer produces at least about 80% respirable fraction. In yet still other embodiments, the system for delivering a therapeutically effective dose of an aqueous inhalation mixture comprising the corticosteroid by said nebulizer produces at least about 85% respirable fraction. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, a solvent and a solubility enhancer and are substantially free of active pharmaceutical agents other than corticosteroid. In still other embodiments, the inhalable compositions comprises an effective amount of a budesonide, a solvent and a solubility enhancer and are substantially free of active pharmaceutical agents other than the budesonide.

In some embodiments of this invention, the system comprises an aqueous inhalation mixture comprising an amount of corticosteroid in mixture prior to administration of about 15 to about 2000 μg of a corticosteroid. In other embodiments, the inhalation mixtures comprise an amount of corticosteroid in mixture prior to administration of about 250 to about 2000 μg of a corticosteroid. In still other embodiments, the inhalation mixtures comprise an amount of corticosteroid in mixture prior to administration of about 60 to about 1500 μg of a corticosteroid. In yet other embodiments, the inhalation mixtures comprise an amount of corticosteroid in mixture prior to administration of about 100 to about 1000 μg of a corticosteroid. In still other embodiments, the inhalation mixtures comprise an amount of corticosteroid in mixture prior to administration of about 120 to about 1000 μg of a corticosteroid. In yet still other embodiments, the inhalation mixtures comprise an amount of corticosteroid in mixture prior to administration of about 125 to about 500 μg of a corticosteroid. In certain embodiments, the inhalation mixtures comprise an amount of corticosteroid in mixture prior to administration of about about 40, about 60, about 100, about 120, about 125, about 240, about 250, about 500, about 1000, about 1500, or about 2000 μg of a corticosteroid. In one embodiment, the inhalation mixture comprises an amount of corticosteroid in mixture prior to administration of about 40 μg of a corticosteroid. In another embodiment, the inhalation mixture comprises an amount of corticosteroid in mixture prior to administration of about 60 μg of a corticosteroid. In still another embodiment, the inhalation mixture comprises an amount of corticosteroid in mixture prior to administration of about 100 μg of a corticosteroid. In yet another embodiment, the inhalation mixture comprises an amount of corticosteroid in mixture prior to administration of about 120 μg of a corticosteroid. In yet still another embodiment, the inhalation mixture comprises an amount of corticosteroid in mixture prior to administration of about 125 μg of a corticosteroid. In still another embodiment, the inhalation mixture comprises an amount of corticosteroid in mixture prior to administration of about 240 μg of a corticosteroid. In still yet another embodiment, the inhalation mixture comprises an amount of corticosteroid in mixture prior to administration of less than about 250 μg of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, and a solubility enhancer and are substantially free of active pharmaceutical agents other than corticosteroid. In still other embodiments, the inhalable compositions comprises an effective amount of a budesonide, and a solubility enhancer and are substantially free of active pharmaceutical agents other than the budesonide.

In some embodiments, suitable aqueous inhalation mixture comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer. In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

The corticosteroids that are useful in the inhalation mixtures described herein included, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone; fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In preferred embodiments, the corticosteroid is budesonide. In other preferred embodiments, the corticosteroid is budesonide wherein the budesonide is either an individual diastereomer or a mixture of the two diastereomers administered individually or together for a therapeutic effect.

In certain embodiments, the systems and methods described herein comprise a solvent. In certain embodiments, the solvent is selected from the group consisting of water, water/ethanol mixture, aqueous alcohol, propylene glycol, or aqueous organic solvent, or combinations thereof. In certain embodiments, the solvent comprises water. In preferred embodiments, the solvent is water.

In other embodiments of the inhalation mixtures described herein, the inhalation mixture comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

In other embodiments, the inhalation mixture for use in the present methods further comprises a solubility enhancer. In certain embodiments, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In preferred embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain embodiments, the solubility enhancer is SBE7-β-CD (Captisol®).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein.

In still other embodiments, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain other embodiments, the nebulizer is a Pari eFlow nebulizer.

An aspect of this invention relates to an inhalation system for delivering a therapeutically effective dose of a corticosteroid to a patient comprising (a) an inhalable aqueous mixture comprising the corticosteroid and a solubility enhancer, and (b) a nebulizer, whereby upon administration of said inhalable aqueous mixture through said nebulizer, the system delivers an enhanced lung deposition of the corticosteroid as compared to an inhalable suspension comprising a corticosteroid administered under the same conditions. In a preferred embodiment of this invention, the system achieves at least about 10% higher respirable fraction compared to an inhalable suspension comprising the corticosteroid administered under the same conditions. In a more preferred embodiment of this invention, the system achieves at least about 15% higher respirable fraction compared to an inhalable suspension comprising the corticosteroid administered under the same conditions. In the most preferred embodiment of this invention, the system achieves at least about 20% higher respirable fraction compared to an inhalable suspension comprising the corticosteroid administered under the same conditions.

In a preferred embodiment of this invention, the system comprising an inhalable aqueous mixture comprising a corticosteroid and a solubility enhancer achieves at least about 5% higher lung deposition compared to an inhalable suspension comprising the corticosteroid administered under the same conditions. In another preferred embodiment of this invention, the system comprising an inhalable aqueous mixture comprising a corticosteroid and a solubility enhancer achieves at least about 10% higher lung deposition compared to an inhalable suspension comprising the corticosteroid administered under the same conditions. In a more preferred embodiment of this invention, the system comprising an inhalable aqueous mixture comprising a corticosteroid and a solubility enhancer achieves at least about 15% higher lung deposition compared to an inhalable suspension comprising the corticosteroid administered under the same conditions. In another more preferred embodiment of this invention, the system comprising an inhalable aqueous mixture comprising a corticosteroid and a solubility enhancer achieves at least about 20% higher lung deposition compared to an inhalable suspension comprising the corticosteroid administered under the same conditions. In the most preferred embodiment of this invention, the system comprising an inhalable aqueous mixture comprising a corticosteroid and a solubility enhancer achieves at least about 25% higher lung deposition compared to an inhalable suspension comprising the corticosteroid administered under the same conditions.

In some embodiments of this invention, the system comprising an inhalable aqueous mixture comprising a corticosteroid and a solubility enhancer achieves about the same lung deposition of the corticosteroid as compared to an inhalable suspension comprising the corticosteroid, wherein the composition is administered at a lower nominal dosage than the inhalable suspension. In some embodiments of this invention, the system comprising an inhalable aqueous mixture comprising a corticosteroid and a solubility enhancer achieves about 90% to 110% lung deposition of the corticosteroid as compared to an inhalable suspension comprising the corticosteroid. In some embodiments of this invention, the system comprising an inhalable aqueous mixture comprising a corticosteroid and a solubility enhancer achieves about 80% to 120% lung deposition of the corticosteroid as compared to an inhalable suspension comprising the corticosteroid. In some embodiments of this invention, the system comprising an inhalable aqueous mixture comprising a corticosteroid and a solubility enhancer achieves about 70% to 130% lung deposition of the corticosteroid as compared to an inhalable suspension comprising the corticosteroid.

The systems described herein can deliver an inhalable aqueous mixture comprising a corticosteroid, e.g., budesonide, a solvent and a solubility enhancer to the subject in a manner wherein the active is delivered in accordance with good medical practice, taking into account the clinical condition of the individual patient, the site and method of administration, scheduling of administration, and other factors known to medical practitioners. In human therapy, the systems and methods described herein can deliver inhalable aqueous mixtures comprising corticosteroids, e.g., a budesonide solution, that maintain a therapeutically effective amount of the corticosteroid, e.g., budesonide, at the site of action which reduces or mitigates symptoms related to bronchoconstrictive disorders.

In another aspect, the inhalable aqueous mixture comprises a corticosteroid, such as budesonide, wherein the inhalable aqueous mixture is administered according to the methods described herein no more than twice a day (b.i.d.). In yet another aspect, the inhalable aqueous mixture comprises a corticosteroid, such as budesonide, wherein the inhalable aqueous mixture is administered according to the methods described herein no more than once a day. In still another embodiment, the inhalable aqueous mixture comprises a corticosteroid, such as budesonide, wherein the inhalable aqueous mixture is administered no more than once a day in the evening.

In still other embodiments, the systems of the present invention can deliver a therapeutically effective amount of a corticosteroid in a significantly shorter period of time than conventional inhalable corticosteroid therapies. For example, the nebulization time for Pulmicort® Respules administered by a Pari LC Plus jet nebulizer takes at least 5 minutes to 8 minutes, and in some cases in excess of 10 minutes. By contrast, the methods and systems of the present invention can deliver a therapeutically effective amount of a corticosteroid, such as a budesonide, over a delivery time of less than about 5 minutes to less than about 1.5 minutes. In some embodiments, the delivery time can be about 5 minutes. In other embodiments, the delivery time can be less than about 5 minutes. In certain embodiments, the delivery time can be about 4.5 minutes. In certain other embodiments, the delivery time can be less than about 4.5 minutes. In still other embodiments, the delivery time can be about 4 minutes. In yet other embodiments, the delivery time can be less than about 4 minutes. In still yet other embodiments, the delivery time can be about 3.5 minutes. In other embodiments, the delivery time can be less than about 3.5 minutes. In yet still other embodiments, the delivery time can be about 3 minutes. In other embodiments, the delivery time can be less than about 3 minutes. In certain embodiments, the delivery time can be about 2.5 minutes. In other certain embodiments, the delivery time can be less than about 2.5 minutes. In still other embodiments, the delivery time can be about 2 minutes. In yet still other embodiments, the delivery time can be less than about 2 minutes. In a preferred embodiment, the delivery time can be about 1.5 minutes. In a more preferred embodiment, the delivery time can be less than about 1.5 minutes.

As previously stated, the nebulization time for Pulmicort® Respules administered by a Pari LC Plus jet nebulizer can take in excess of 10 minutes. This prolonged administration time is very burdensome on the patient, especially when the patient is a pediatric patient. Thus, a system or method that can reduce the time of delivery of a corticosteroid by inhalation can increase the patient's compliance with the therapeutic regimen. By contrast, the methods and systems of the present invention can deliver a therapeutically effective amount of a corticosteroid, such as a budesonide, over a delivery time of less than about 5 minutes to less than about 1.5 minutes. In some embodiments, the delivery time can be about 5 minutes. In other embodiments, the delivery time can be less than about 5 minutes. In certain embodiments, the delivery time can be about 4.5 minutes. In certain other embodiments, the delivery time can be less than about 4.5 minutes. In still other embodiments, the delivery time can be about 4 minutes. In yet other embodiments, the delivery time can be less than about 4 minutes. In still yet other embodiments, the delivery time can be about 3.5 minutes. In other embodiments, the delivery time can be less than about 3.5 minutes. In yet still other embodiments, the delivery time can be about 3 minutes. In other embodiments, the delivery time can be less than about 3 minutes. In certain embodiments, the delivery time can be about 2.5 minutes. In other certain embodiments, the delivery time can be less than about 2.5 minutes. In still other embodiments, the delivery time can be about 2 minutes. In yet still other embodiments, the delivery time can be less than about 2 minutes. In a preferred embodiment, the delivery time can be about 1.5 minutes. In a more preferred embodiment, the delivery time can be less than about 1.5 minutes.

In still other embodiments of this invention, the inhalation mixture for use in the present methods further comprises a second therapeutic agent selected from the group consisting of a beta2-adrenoreceptor agonist, a prophylactic therapeutic, and an anti-cholinergic agent. In other embodiments of this invention, the beta2-adrenoreceptor agonist is albuterol, levalbuterol or a pharmaceutical acceptable derivative.

Another aspect of this invention relates to an inhalation system for delivering a therapeutically effective dose of albuterol to a patient comprising (a) an aqueous inhalation mixture comprising albuterol, and (b) a Pari eFlow nebulizer, whereby delivering said inhalation mixture by said nebulizer delivers an enhanced lung deposition of the corticosteroid as compared to albuterol with another nebulizer administered under the same conditions. Also an aspect of this invention relates to an inhalation system for delivering a therapeutically effective dose of albuterol to a patient comprising (a) an aqueous inhalation mixture comprising albuterol, and (b) a Pari eFlow nebulizer, whereby delivering said inhalation mixture by said nebulizer delivers an enhanced pharmacokinetic profile of the corticosteroid as compared to albuterol administered with another nebulizer under the same conditions. In some embodiments of this invention, the inhalable composition comprises a corticosteroid.

VII. Corticosteroid Inhalation Systems which Provide a Decreased Increase in the Concentration of the Corticosteroid within a Device

Another aspect of this invention relates to an inhalation system for delivering a therapeutically effective dose of a corticosteroid to a patient comprising (a) an aqueous inhalation mixture comprising the corticosteroid, a solvent and a solubility enhancer, and (b) a nebulizer, whereby upon administration of the composition to a subject through a nebulizer, the system achieves rate of increasing concentration of the corticosteroid inside the device of about 60% or less or a rate of increasing concentration of the corticosteroid inside the device achieved by an inhalable suspension comprising the corticosteroid without a solubility enhancer administered under the same conditions. In certain embodiments, the aqueous inhalation mixture comprises a single corticosteroid and is substantially free of active pharmaceutical agents other than the corticosteroid.

In certain embodiments, the rate of increasing concentration of the corticosteroid inside the device is achieved over the first 3 minutes of administration. In other embodiments, the rate of increasing concentration of the corticosteroid inside the device is achieved during the second and third minute of administration. In still other embodiments, the rate of increasing concentration of the corticosteroid inside the device is achieved during the third minute of administration.

In one embodiment, the invention relates to an inhalable composition wherein administration of the composition through the device is achieved over five minutes or less, and administration of the inhalable suspension is achieved over five minutes or less. In another embodiment, the invention relates to an inhalable composition wherein the time of administration of the composition through the device and the time of administration of the inhalable suspension are the same. In still other embodiments, the invention relates to an inhalable composition wherein the time of administration of the composition through the device and the time of administration of the inhalable suspension are different.

In certain embodiments of this invention, the inhalable composition also achieves at least about 60% respirable fraction upon administration. In a more preferred embodiment of this invention, the inhalable composition also achieves at least about 70% respirable fraction upon administration. In a still more preferred embodiment of this invention, the inhalable composition also achieves at least about 80% respirable fraction upon administration. In the most preferred embodiment of this invention, the inhalable composition also achieves at least about 85% respirable fraction upon administration.

In some embodiments of this invention, the system comprises an aqueous inhalation mixture comprising about 15 to about 2000 μg of a corticosteroid. In other embodiments, the inhalation mixture comprises about 50 to about 2000 μg of a corticosteroid. In still other embodiments, the inhalation mixtures comprise about 60 to about 1500 μg of a corticosteroid. In yet other embodiments, the inhalation mixtures comprise about 100 to about 1000 μg of a corticosteroid. In still other embodiments, the inhalation mixtures comprising comprise about 120 to about 1000 μg of a corticosteroid. In yet still other embodiments, the inhalation mixtures comprise about 125 to about 500 μg of a corticosteroid. In certain embodiments, the inhalation mixtures comprise about 40, 60, 100, 120, 125, 240; 250, 500, 1000, 1500, or 2000 μg of a corticosteroid. In one embodiment, the inhalation mixture comprises a nominal dosage of about 40 μg of a corticosteroid. In another embodiment, the inhalation mixture comprises a nominal dosage of about 60 μg of a corticosteroid. In yet another embodiment, the inhalation mixture comprises a nominal dosage of about 100 μg of a corticosteroid. In yet still another embodiment, the inhalation mixture comprises a nominal dosage of about 120 μg of a corticosteroid. In still another embodiment, the inhalation mixture comprises a nominal dosage of about 125 μg of a corticosteroid. In still yet another embodiment, the inhalation mixture comprises a nominal dosage of about 240 μg of a corticosteroid. In still yet another embodiment, the inhalation mixture comprises a nominal dosage of less than about 250 μg of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalation mixtures comprise an effective amount of a single corticosteroid, a solvent and a solubility enhancer and are substantially free of active pharmaceutical agents other than corticosteroid. In still other embodiments, the inhalation mixtures comprise an effective amount of a budesonide, a solvent and a solubility enhancer and are substantially free of active pharmaceutical agents other than the budesonide.

In some embodiments, suitable aqueous inhalation mixture comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

The corticosteroids that are useful in the inhalation mixtures described herein included, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In preferred embodiments, the corticosteroid is budesonide. In other preferred embodiments, the corticosteroid is budesonide wherein the budesonide is either an individual diastereomer or a mixture of the two diastereomers administered individually or together for a therapeutic effect.

In certain embodiments, the systems and methods described herein comprise a solvent. In certain embodiments, the solvent is selected from the group consisting of water, water/ethanol mixture, aqueous alcohol, propylene glycol, or aqueous organic solvent, or combinations thereof. In certain embodiments, the solvent comprises water. In preferred embodiments, the solvent is water.

In certain embodiments of the inhalation mixtures described herein, the inhalation mixture comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

In some embodiments of this invention, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-7-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain embodiments, the solubility enhancer is SBE7-β-CD (Captisol®).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein.

In some embodiments of this invention, the composition further comprises a second therapeutic agent selected from the group consisting of a beta2-adrenoreceptor agonist, a prophylactic therapeutic, and an anti-cholinergic agent. In some embodiments of this invention, the beta2-adrenoreceptor agonist is albuterol, levalbuterol or a pharmaceutical acceptable derivative.

In another aspect, the inhalable aqueous mixture comprises a corticosteroid, such as budesonide, wherein the inhalable aqueous mixture is administered according to the methods described herein no more than twice a day (b.i.d.). In yet another aspect, the inhalable aqueous mixture comprises a corticosteroid, such as budesonide, wherein the inhalable aqueous mixture is administered according to the methods described herein no more than once a day. In still another embodiment, the inhalable aqueous mixture comprises a corticosteroid, such as budesonide, wherein the inhalable aqueous mixture is administered no more than once a day in the evening.

In some embodiments of this invention, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain embodiments, the nebulizer is a Pari eFlow nebulizer.

VIII. Methods of Treatment to Achieve Enhanced Lung Deposition

In other aspects of the present invention, methods are provided for the delivery of a therapeutically effective dose of a corticosteroid to a patient. In certain embodiments, the methods described herein are directed to the treatment of a bronchoconstrictive disorder in a patient comprising providing an inhalable aqueous mixture comprising a corticosteroid, a solvent and a solubility enhancer and delivering the aqueous inhalation mixture via an inhalation nebulizer.

In certain embodiments, the present invention can provide a method for the treatment of a bronchoconstrictive disorder in a patient in need of treatment thereof comprising forming a mixture by adding a solvent and a solubility enhancer to an amount of corticosteroid and operating a nebulizer, wherein upon administration of the mixture to a subject through the nebulizer, the methods can achieves at least about 20% to about 40%, between about 20% to about 50%, or between about 20% to about 55% lung deposition e.g., bronchi and alveoli, based on the amount of corticosteroid in the mixture prior to administration. In some embodiments, the methods can achieve at least about 20% to about 35% lung deposition based on the amount of corticosteroid in the mixture prior to administration. In other embodiments, the methods can achieve at least 20% to about 30% lung deposition based on the amount of corticosteroid in the mixture prior to administration. In certain embodiments, the methods achieve about 25% lung deposition based on the amount of corticosteroid in the composition prior to administration. In other embodiments, the methods achieve at least about 30% lung deposition based on the amount of corticosteroid in the composition prior to administration. In still other embodiments, the methods achieve at least about 35% lung deposition based on the amount of corticosteroid in the composition prior to administration. In yet other embodiments, the methods achieve at least about 40% lung deposition based on the amount of corticosteroid in the composition prior to administration. In yet still other embodiments, the methods achieve at least about 45% lung deposition based on the amount of corticosteroid in the composition prior to administration. In still yet other embodiments, the methods achieve at least about 50% lung deposition based on the amount of corticosteroid in the composition prior to administration. In other embodiments, the methods achieve at least about 40% to about 55% lung deposition based on the amount of corticosteroid in the composition prior to administration. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, and a solubility enhancer and are substantially free of active pharmaceutical agents other than corticosteroid. In still other embodiments, the inhalable compositions comprises an effective amount of budesonide, and a solubility enhancer and are substantially free of active pharmaceutical agents other than the budesonide.

In other embodiments of this invention, the methods also achieve at least about 60% respirable fraction upon administration. In more preferred embodiments of this invention, the methods also achieve at least about 70% respirable fraction upon administration. In still more preferred embodiments of this invention, the methods also achieve at least about 80% respirable fraction upon administration. In the most preferred embodiments of this invention, the methods also achieve at least about 85% respirable fraction upon administration. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, and a solubility enhancer and are substantially free of active pharmaceutical agents other than corticosteroid. In still other embodiments, the inhalable compositions comprises an effective amount of a budesonide, and a solubility enhancer and are substantially free of active pharmaceutical agents other than the budesonide.

In certain embodiments, the methods of treatment of a bronchoconstrictive disorder comprise the delivery of an inhalable aqueous mixture comprising a corticosteroid. The corticosteroids that are useful is in the present invention include, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In some embodiments, the corticosteroid is budesonide. In other embodiments, the corticosteroid is budesonide wherein the budesonide is either an individual diastereomer or a mixture of the two diastereomers administered individually or together for a therapeutic effect.

In some embodiments of this invention, the methods of treatment comprise an aqueous inhalation mixture comprising an amount of corticosteroid in mixture prior to administration of about 15 to about 2000 μg of a corticosteroid. In other embodiments, the inhalation mixtures comprise an amount of corticosteroid in mixture prior to administration of about 250 to about 2000 μg of a corticosteroid. In still other embodiments, the inhalation mixtures comprise an amount of corticosteroid in mixture prior to administration of about 60 to about 1500 μg of a corticosteroid. In yet other embodiments, the inhalation mixtures comprise an amount of corticosteroid in mixture prior to administration of about 100 to about 1000 μg of a corticosteroid. In still other embodiments, the inhalation mixtures comprise an amount of corticosteroid in mixture prior to administration of about 120 to about 1000 μg of a corticosteroid. In yet still other embodiments, the inhalation mixtures comprise an amount of corticosteroid in mixture prior to administration of about 125 to about 500 μg of a corticosteroid. In certain embodiments, the inhalation mixtures comprise an amount of corticosteroid in mixture prior to administration of about about 40, about 60, about 100, about 120, about 125, about 240, about 250, about 500, about 1000, about 1500, or about 2000 μg of a corticosteroid. In one embodiment, the inhalation mixture comprises an amount of corticosteroid in mixture prior to administration of about 40 μg of a corticosteroid. In another embodiment, the inhalation mixture comprises an amount of corticosteroid in mixture prior to administration of about 60 μg of a corticosteroid. In still another embodiment, the inhalation mixture comprises an amount of corticosteroid in mixture prior to administration of about 100 μg of a corticosteroid. In yet another embodiment, the inhalation mixture comprises an amount of corticosteroid in mixture prior to administration of about 120 μg of a corticosteroid. In yet still another embodiment, the inhalation mixture comprises an amount of corticosteroid in mixture prior to administration of about 125 μg of a corticosteroid. In still another embodiment, the inhalation mixture comprises an amount of corticosteroid in mixture prior to administration of about 240 μg of a corticosteroid. In still yet another embodiment, the inhalation mixture comprises an amount of corticosteroid in mixture prior to administration of less than about 250 μg of a corticosteroid. In one embodiment, the corticosteroid is budesonide. In another embodiment, the corticosteroid is budesonide wherein the budesonide is either an individual diastereomer or a mixture of the two diastereomers administered individually or together for a therapeutic effect. In certain embodiments, the inhalable compositions comprise an effective amount of a single corticosteroid, a solvent and a solubility enhancer and are substantially free of active pharmaceutical agents other than corticosteroid. In still other embodiments, the inhalable compositions comprises an effective amount of a budesonide, a solvent and a solubility enhancer and are substantially free of active pharmaceutical agents other than the budesonide.

In certain embodiments, the inhalable mixtures can comprise about 40 μg budesonide, a solvent and a solubility enhancer, wherein upon administration of the composition to a subject through a nebulizer, the composition achieves lung deposition of at least 13 μg of budesonide. In certain other embodiments, the inhalable mixtures can comprise about 60 μg budesonide, a solvent and a solubility enhancer, wherein upon administration of the composition to a subject through a nebulizer, the composition achieves lung deposition of at least 20 μg of budesonide. In still other embodiments, the inhalable composition can comprise about 120 μg budesonide, a solvent and a solubility enhancer, wherein upon administration of the composition to a subject through a nebulizer, the composition achieves lung deposition of at least 40 μg of budesonide. In yet other embodiments, the inhalable composition can comprise about 240 μg budesonide, a solvent and a solubility enhancer, wherein upon administration of the composition to a subject through a nebulizer, the composition achieves lung deposition of at least 80 μg of budesonide.

In certain embodiments, the inhalable mixtures can comprise about 40 μg budesonide, a solvent and a solubility enhancer, wherein upon administration of the composition to a subject through a nebulizer, the composition achieves lung deposition of at least 13 μg of budesonide, wherein the composition is substantially free of active pharmaceutical agents other than the budesonide. In certain other embodiments, the inhalable mixtures can comprise about 60 μg budesonide, a solvent and a solubility enhancer, wherein upon administration of the composition to a subject through a nebulizer, the composition achieves lung deposition of at least 20 μg of budesonide, wherein the composition is substantially free of active pharmaceutical agents other than the budesonide. In still other embodiments, the inhalable composition can comprise about 120 μg budesonide, a solvent and a solubility enhancer, wherein upon administration of the composition to a subject through a nebulizer, the composition achieves lung deposition of at least 40 μg of budesonide, wherein the composition is substantially free of active pharmaceutical agents other than the budesonide. In yet other embodiments, the inhalable composition can comprise about 240 μg budesonide, a solvent and a solubility enhancer, wherein upon administration of the composition to a subject through a nebulizer, the composition achieves lung deposition of at least 80 μg of budesonide, wherein the composition is substantially free of active pharmaceutical agents other than the budesonide.

In some embodiments, suitable aqueous inhalation mixture comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer. In certain embodiments, the systems and methods described herein comprise a solvent. In certain embodiments, the solvent is selected from the group consisting of water, water/ethanol mixture, aqueous alcohol, propylene glycol, or aqueous organic solvent, or combinations thereof. In certain embodiments, the solvent comprises water. In preferred embodiments, the solvent is water.

In some embodiments, the methods of treatment of a bronchoconstrictive disorder comprise the delivery of an inhalable aqueous mixture comprising a corticosteroid and a solvent. In certain embodiments, the solvent is selected from the group consisting of water, aqueous alcohol, propylene glycol, or aqueous organic solvent. In preferred embodiments, the solvent is water.

In certain embodiments of the inhalation mixtures described herein, the inhalation mixture comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

In some embodiments of this invention, the methods of treatment of a bronchoconstrictive disorder comprise the delivery of an inhalable aqueous mixture comprising a corticosteroid and a solubility enhancer. In certain embodiments, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In other embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain embodiments, the solubility enhancer is SBE7-β-CD (Captisol®).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein.

In some embodiments of this invention, the composition further comprises a second therapeutic agent selected from the group consisting of a beta2-adrenoreceptor agonist, a prophylactic therapeutic, and an anti-cholinergic agent. In some embodiments of this invention, the beta2-adrenoreceptor agonist is albuterol, levalbuterol or a pharmaceutical acceptable derivative.

In certain embodiments of this invention, the bronchoconstrictive disorder is selected from the group consisting of asthma, pediatric asthma, bronchial asthma, allergic asthma, intrinsic asthma, chronic obstructive pulmonary disease (COPD), chronic bronchitis, and emphysema.

In another aspect, the inhalable aqueous mixture comprises a corticosteroid, such as budesonide, wherein the inhalable aqueous mixture is administered according to the methods described herein no more than twice a day (b.i.d). In yet another aspect, the inhalable aqueous mixture comprises a corticosteroid, such as budesonide, wherein the inhalable aqueous mixture is administered according to the methods described herein no more than once a day. In still another embodiment, the inhalable aqueous mixture comprises a corticosteroid, such as budesonide, wherein the inhalable aqueous mixture is administered no more than once a day in the evening.

In some embodiments of this invention, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain embodiments, the nebulizer is a Pari eFlow nebulizer.

In still other embodiments, the present invention can provide a method for the treatment of a bronchoconstrictive disorder in a patient comprising providing an inhalable aqueous mixture comprising a corticosteroid, a solvent and a solubility enhancer and delivering the aqueous inhalation mixture via an inhalation nebulizer, wherein the delivery of the inhalable aqueous mixture provides that no more than about 10% to about 30% of the corticosteroid is delivered outside of the lung, e.g., in the mouth, esophagus, and/or stomach. In one embodiment, the method can provide the delivery of an inhalable aqueous mixture wherein no more than about 10% of the corticosteroid is delivered outside of the lung. In another embodiment, the method can provide the delivery of an inhalable aqueous mixture wherein no more than about 15% of the corticosteroid is delivered outside of the lung. In yet another embodiment, the method can provide the delivery of an inhalable aqueous mixture wherein no more than about 20% of the corticosteroid is delivered outside of the lung. In still another embodiment, the method can provide the delivery of an inhalable aqueous mixture wherein no more than about 25% of the corticosteroid is delivered outside of the lung. In yet another embodiment, the method can provide the delivery of an inhalable aqueous mixture wherein no more than about 30% of the corticosteroid is delivered outside of the lung.

In other embodiments, the present invention can provide a method for the prophylaxis of a bronchoconstrictive disorder in a patient comprising providing an inhalable aqueous mixture comprising a corticosteroid, a solvent and a solubility enhancer and delivering the aqueous inhalation mixture via an inhalation nebulizer. In another embodiment, the present invention can provide a method for reducing the risk of side effects associated with corticosteroid inhalation therapy whereby a lower nominal dosage of the corticosteroid is required to achieve a therapeutic effect as compared to conventional inhalable corticosteroid therapies. In one embodiment, the risk of side effects is lowered compared to conventional inhalable corticosteroid therapies.

IX. Methods of Treatment which Provide a Decreased Increase in the Concentration of Corticosteroid within the Device

An additional aspect of this invention relates to a method of the treatment of a bronchoconstrictive disorder in a patient in need of treatment thereof comprising forming a composition by adding a solvent and a solubility enhancer to a corticosteroid and operating a nebulizer, wherein upon administration of the composition to a subject through the nebulizer, the composition achieves rate of increasing concentration of the corticosteroid inside the device of about 60% or less or a rate of increasing concentration of the corticosteroid inside the device achieved by an inhalable suspension comprising the corticosteroid without a solubility enhancer administered under the same conditions. In certain embodiments, the composition comprises a single corticosteroid and is substantially free of active pharmaceutical agents other than the corticosteroid. In other embodiments of this invention, the corticosteroid is budesonide or a pharmaceutical acceptable derivative. In still other embodiments, the corticosteroid is budesonide wherein the budesonide is either an individual diastereomer or a mixture of the two diastereomers administered individually or together for a therapeutic effect.

In certain embodiments, the rate of increasing concentration of the corticosteroid inside the device is achieved over the first 3 minutes of administration. In other embodiments, the rate of increasing concentration of the corticosteroid inside the device is achieved during the second and third minute of administration. In still other embodiments, the rate of increasing concentration of the corticosteroid inside the device is achieved during the third minute of administration.

In one embodiment, the invention relates to an inhalable composition wherein administration of the composition through the device is achieved over five minutes or less, and administration of the inhalable suspension is achieved over five minutes or less. In another embodiment, the invention relates to an inhalable composition wherein the time of administration of the composition through the device and the time of administration of the inhalable suspension are the same. In still other embodiments, the invention relates to an inhalable composition wherein the time of administration of the composition through the device and the time of administration of the inhalable suspension are different.

In certain embodiments of this invention, the inhalable composition also achieves at least about 60% respirable fraction upon administration. In a preferred embodiment of this invention, the inhalable composition also achieves at least about 70% respirable fraction upon administration. In a more preferred embodiment of this invention, the inhalable composition also achieves at least about 80% respirable fraction upon administration. In the most preferred embodiment of this invention, the inhalable composition also achieves at least about 85% respirable fraction upon administration.

In some embodiments of this invention, the system comprises an aqueous inhalation mixture comprising about 15 to about 2000 μg of a corticosteroid. In other embodiments, the inhalation mixtures comprise about 50 to about 2000 μg of a corticosteroid. In still other embodiments, the inhalation mixtures comprise about 60 to about 1500 μg of a corticosteroid. In yet other embodiments, the inhalation mixtures comprise about 100 to about 1000 μg of a corticosteroid. In still other embodiments, the inhalation mixtures comprising comprise about 120 to about 1000 μg of a corticosteroid. In yet still other embodiments, the inhalation mixtures comprise about 125 to about 500 μg of a corticosteroid. In certain embodiments, the inhalation mixtures comprise about 40, 60, 100, 120, 125, 240, 250, 500, 1000, 1500, or 2000 μg of a corticosteroid. In one embodiment, the inhalation mixture comprises a nominal dosage of about 40 μg of a corticosteroid. In another embodiment, the inhalation mixture comprises a nominal dosage of about 60 μg of a corticosteroid. In still another embodiment, the inhalation mixture comprises a nominal dosage of about 100 μg of a corticosteroid. In yet another embodiment, the inhalation mixture comprises a nominal dosage of about 120 μg of a corticosteroid. In yet still another embodiment, the inhalation mixture comprises a nominal dosage of about 125 μg of a corticosteroid. In still another embodiment, the inhalation mixture comprises a nominal dosage of about 240 μg of a corticosteroid. In still yet another embodiment, the inhalation mixture comprises a nominal dosage of less than about 250 μg of a corticosteroid. In other embodiments of this invention, the corticosteroid is budesonide or a pharmaceutical acceptable derivative. In other preferred embodiments, the corticosteroid is budesonide wherein the budesonide is either an individual diastereomer or a mixture of the two diastereomers administered individually or together for a therapeutic effect.

In some embodiments, suitable aqueous inhalation mixture comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

In certain embodiments, the systems and methods described herein comprise a solvent. In certain embodiments, the solvent is selected from the group consisting of water, water/ethanol mixture, aqueous alcohol, propylene glycol, or aqueous organic solvent, or combinations thereof. In certain embodiments, the solvent comprises water. In preferred embodiments, the solvent is water.

In certain embodiments of the inhalation mixtures described herein, the inhalation mixture comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

In some embodiments of this invention, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®)), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain other embodiments, the solubility enhancer is SBE7-β-CD (Captisol®).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein.

In some embodiments of this invention, the composition further comprises a second therapeutic agent selected from the group consisting of a beta2-adrenoreceptor agonist, a prophylactic therapeutic, and an anti-cholinergic agent. In some embodiments of this invention, the beta2-adrenoreceptor agonist is albuterol, levalbuterol or a pharmaceutical acceptable derivative.

In another aspect, the inhalable aqueous mixture comprises a corticosteroid, such as budesonide, wherein the inhalable aqueous mixture is administered according to the methods described herein no more than twice a day (b.i.d). In yet another aspect, the inhalable aqueous mixture comprises a corticosteroid, such as budesonide, wherein the inhalable aqueous mixture is administered according to the methods described herein no more than once a day. In still another embodiment, the inhalable aqueous mixture comprises a corticosteroid, such as budesonide, wherein the inhalable aqueous mixture is administered no more than once a day in the evening.

In some embodiments of this invention, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain embodiments, the nebulizer is a Pari eFlow nebulizer.

In certain embodiments of this invention, the bronchoconstrictive disorder is selected from the group consisting of asthma, pediatric asthma, bronchial asthma, allergic asthma, intrinsic asthma, chronic obstructive pulmonary disease (COPD), chronic bronchitis, and emphysema.

X. Methods of Manufacturing the Inhalable Compositions of the Present Invention

Another aspect of this invention relates to use of a corticosteroid in the manufacture of an inhalable composition for the treatment or prophylaxis of a bronchoconstrictive disorder in a patient in need of treatment thereof, comprising adding a solvent and a solubility enhancer to an amount of corticosteroid and operating a nebulizer, wherein the composition achieves at least about 25% lung deposition based on the amount of corticosteroid in the composition prior to administration. In some embodiments of this invention, the composition also achieves at least about 60% respirable fraction upon administration. In certain embodiments, the inhalable compositions comprise a single corticosteroid, a solvent and a solubility enhancer and are substantially free of active pharmaceutical agents other than corticosteroids. In some embodiments, the corticosteroid is budesonide. In other embodiments, the corticosteroid is budesonide wherein the budesonide is either an individual diastereomer or a mixture of the two diastereomers administered individually or together for a therapeutic effect.

An aspect of this invention also relates to use of a corticosteroid in the manufacture of an inhalable composition for the treatment of a bronchoconstrictive disorder in a patient in need of treatment thereof, comprising adding a solvent and a solubility enhancer to an amount of corticosteroid and operating a nebulizer, wherein the composition achieves at least about 5% higher lung deposition compared to an inhalable suspension comprising the corticosteroid administered under the same conditions to deliver a therapeutically effective amount of said corticosteroid. In certain embodiments, the inhalable compositions comprise a single corticosteroid, a solvent and a solubility enhancer and are substantially free of active pharmaceutical agents other than corticosteroids.

An aspect of this invention also relates to use of a corticosteroid in the manufacture of an inhalable composition for the treatment of a bronchoconstrictive disorder in a patient in need of treatment thereof, comprising adding a solvent and a solubility enhancer to an amount of corticosteroid and operating a nebulizer, wherein the composition achieves about the same lung deposition compared to an inhalable suspension comprising the corticosteroid, wherein the composition is administered at a lower nominal dosage than the inhalable suspension to deliver a therapeutically effective amount of said corticosteroid. In certain embodiments, the inhalable compositions comprise a single corticosteroid, a solvent and a solubility enhancer and are substantially free of active pharmaceutical agents other than corticosteroids.

In some embodiments of this invention, the system comprises an aqueous inhalation mixture comprising a corticosteroid with about 15 to about 2000 μg of a corticosteroid. In other embodiments, the inhalation mixture comprises about 50 to about 2000 μg of a corticosteroid. In still other embodiments, the inhalation mixtures comprise about 60 to about 1500 μg of a corticosteroid. In yet other embodiments, the inhalation mixtures comprise about 100 to about 1000 μg of a corticosteroid. In still other embodiments, the inhalation mixtures comprising comprise about 120 to about 1000 μg of a corticosteroid. In yet still other embodiments, the inhalation mixtures comprise about 125 to about 500 μg of a corticosteroid. In certain embodiments, the inhalation mixtures comprise about 40, 60, 100, 120, 125, 240, 250, 500, 1000, 1500, or 2000 μg of a corticosteroid. In one embodiment, the inhalation mixture comprises a nominal dosage of about 40 μg of a corticosteroid. In another embodiment, the inhalation mixture comprises a nominal dosage of about 60 μg of a corticosteroid. In still another embodiment, the inhalation mixture comprises a nominal dosage of about 100 μg of a corticosteroid. In yet another embodiment, the inhalation mixture comprises a nominal dosage of about 120 μg of a corticosteroid. In yet still another embodiment, the inhalation mixture comprises a nominal dosage of about 125 μg of a corticosteroid. In still another embodiment, the inhalation mixture comprises a nominal dosage of about 240 μg of a corticosteroid. In still yet another embodiment, the inhalation mixture comprises a nominal dosage of less than about 250 μg of a corticosteroid. In other embodiments of this invention, the corticosteroid is budesonide or a pharmaceutical acceptable derivative. In other preferred embodiments, the corticosteroid is budesonide wherein the budesonide is either an individual diastereomer or a mixture of the two diastereomers administered individually or together for a therapeutic effect.

In certain embodiments, the systems and methods described herein comprise a solvent. In certain embodiments, the solvent is selected from the group consisting of water, water/ethanol mixture, aqueous alcohol, propylene glycol, or aqueous organic solvent, or combinations thereof. In certain embodiments, the solvent comprises water. In preferred embodiments, the solvent is water.

In some embodiments of this invention, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer is SBE7-β-CD (Captisol®).

In certain other embodiments, the inhalable compositions of the present invention comprises a solubility enhancer is selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain embodiments, the solubility enhancer is SBE7-β-CD (Captisol®).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein.

In some embodiments of this invention, the nebulizer is a jet nebulizer, an ultrasonic nebulizer, a pulsating membrane nebulizer, a nebulizer with a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain other embodiments, the nebulizer is a Pari eFlow nebulizer.

In some embodiments of this invention, the composition further comprises a second therapeutic agent selected from the group consisting of a beta2-adrenoreceptor agonist, a prophylactic therapeutic, and an anti-cholinergic agent. In some embodiments of this invention, the beta2-adrenoreceptor agonist is albuterol, levalbuterol or a pharmaceutical acceptable derivative.

In some embodiments of this invention, the bronchoconstrictive disorder is selected from the group consisting of asthma, pediatric asthma, bronchial asthma, allergic asthma, intrinsic asthma, chronic obstructive pulmonary disease (COPD), chronic bronchitis, and emphysema.

In another aspect, the inhalable compositions comprise a corticosteroid, such as budesonide, wherein the inhalable composition is administered according to the methods described herein no more than twice a day (b.i.d). In yet another aspect, the inhalable composition comprises a corticosteroid, such as budesonide, wherein the inhalable composition is administered according to the methods described herein no more than once a day. In still another embodiment, the inhalable composition comprises a corticosteroid, such as budesonide, wherein the inhalable composition is administered no more than once a day in the evening.

XI. Methods of Treatment with Enhanced pK Profiles

The methods and systems for the treatment or prophylaxis of a bronchoconstrictive disorder in a patient in need thereof described herein can provide enhanced pharmacokinetic profiles for the delivered corticosteroid as compared to a corticosteroid administered via inhalation in the form of a suspension. In preferred embodiments, the methods and systems for the treatment or prophylaxis of a bronchoconstrictive disorder in a patient in need thereof described herein can enable increased local bioavailability of the delivered corticosteroid as compared to conventional inhalation therapies and further provide, inter alia, a means for reducing the dosage required to provide a local therapeutic effect. Likewise provided are methods and systems for the treatment of bronchoconstrictive disorders, e.g., asthma, that can enable the delivery of a corticosteroid having enhanced pharmacokinetic properties as compared to a corticosteroid administered via inhalation in the form of a suspension, wherein the administration by the methods and systems described herein provides one or more of the following advantages: an increase in the local bioavailability of the delivered corticosteroid; a method to reduce the nominal dosage of a corticosteroid required to provide a local therapeutic effect; a method to reduce the time required to administer an effective dose of the corticosteroid; a method to increase patient compliance with a therapeutic regimen comprising inhalation of nebulized corticosteroids; a method of enhanced delivery of a corticosteroid; a method for increasing the amount of corticosteroid deposited in the lung, e.g., bronchi and alveoli; and a method for reducing the side effects associated with inhalation of corticosteroids.

In some embodiments, the methods and systems for the treatment or prophylaxis of a bronchoconstrictive disorder in a patient in need thereof can provide a method for the treatment of a bronchoconstrictive disorder in a patient comprising providing an aqueous inhalation mixture comprising a corticosteroid and a solubility enhancer and delivering the aqueous inhalation mixture with an inhalation nebulizer wherein the corticosteroid is administered at nominal dosage of less than about 250 μg/dose. In one embodiment, the corticosteroid can be administered at nominal dosage of less than about 240 μg/dose. In another embodiment, the corticosteroid can be administered at nominal dosage of less than about 200 μg/dose. In yet another embodiment, the corticosteroid can be administered at nominal dosage of less than about 150 μg/dose. In still another embodiment, the corticosteroid can be administered at nominal dosage of less than about 125 μg/dose. In another embodiment, the corticosteroid can be administered at nominal dosage of about 120 μg/dose. In yet still anther embodiment, the corticosteroid can be administered at nominal dosage of about 100 μg/dose. In yet another embodiment, the corticosteroid can be administered at nominal dosage of about 60 μg/dose. In yet still another embodiment, the corticosteroid can be administered at nominal dosage of about 50 μg/dose. In still another embodiment, the corticosteroid can be administered at nominal dosage of about 40 μg/dose. In certain other embodiments of the methods described herein, the aqueous inhalation mixture can comprise a corticosteroid nominal dosage ranging from about 15 μg/dose to about 250 μg/dose, or about 40 μg/dose to about 250 μg/dose, or about 60 μg/dose to about 250 μg/dose, or about 40 μg/dose to about 200 μg/dose, or about 60 μg/dose to about 200 μg/dose, or about 40 μg/dose to about 150 μg/dose, or about 60 μg/dose to about 150 μg/dose, or about 40 μg/dose to about 125 μg/dose, or about 60 μg/dose to about 125 μg/dose, or about 40 μg/dose to about 100 μg/dose, or about 60 μg/dose to about 100 μg/dose, or about 25 μg/dose to about 50 μg/dose, or about 25 μg/dose to about 60 μg/dose. In some embodiments, the corticosteroid is budesonide. In other embodiments, the corticosteroid is budesonide wherein the budesonide is either an individual diastereomer or a mixture of the two diastereomers administered individually or together for a therapeutic effect.

In other embodiments, the methods and systems for the treatment or prophylaxis of a bronchoconstrictive disorder in a patient in need thereof can provide a method for the treatment of a bronchoconstrictive disorder in a patient comprising providing an aqueous inhalation mixture comprising a single corticosteroid and a solubility enhancer and delivering the aqueous inhalation mixture with an inhalation nebulizer wherein the corticosteroid is administered at nominal dosage of less than about 250 μg/dose and the inhalation mixture is substantially free of pharmaceutically active agents other than the corticosteroid. In one embodiment, the corticosteroid can be administered at nominal dosage of less than about 240 μg/dose and the inhalation mixture is substantially free of pharmaceutically active agents other than the corticosteroid. In another embodiment, the corticosteroid can be administered at nominal dosage of less than about 200 μg/dose and the inhalation mixture is substantially free of pharmaceutically active agents other than the corticosteroid. In yet another embodiment, the corticosteroid can be administered at nominal dosage of less than about 150 μg/dose and the inhalation mixture is substantially free of pharmaceutically active agents other than the corticosteroid. In still another embodiment, the corticosteroid can be administered at nominal dosage of less than about 125 μg/dose and the inhalation mixture is substantially free of pharmaceutically active agents other than the corticosteroid. In another embodiment, the corticosteroid can be administered at nominal dosage of 120 μg/dose and the inhalation mixture is substantially free of pharmaceutically active agents other than the corticosteroid. In yet still anther embodiment, the corticosteroid can be administered at nominal dosage of about 100 μg/dose and the inhalation mixture is substantially free of pharmaceutically active agents other than the corticosteroid. In yet another embodiment, the corticosteroid can be administered at nominal dosage of about 60 μg/dose and the inhalation mixture is substantially free of pharmaceutically active agents other than the corticosteroid. In yet still another embodiment, the corticosteroid can be administered at nominal dosage of about 50 μg/dose and the inhalation mixture is substantially free of pharmaceutically active agents other than the corticosteroid. In another embodiment, the corticosteroid can be administered at nominal dosage of about 40 μg/dose and the inhalation mixture is substantially free of pharmaceutically active agents other than the corticosteroid. In certain other embodiments of the methods described herein, the aqueous inhalation mixture can comprise a corticosteroid nominal dosage ranging from about 15 μg/dose to about 250 μg/dose, or about 40 μg/dose to about 250 μg/dose, or about 60 μg/dose to about 250 μg/dose, or about 40 μg/dose to about 200 μg/dose, or about 60 μg/dose to about 200 μg/dose, or about 40 μg/dose to about 150 μg/dose, or about 60 μg/dose to about 150 μg/dose, or about 40 μg/dose to about 125 μg/dose, or about 60 μg/dose to about 125 μg/dose, or about 40 μg/dose to about 100 μg/dose, or about 60 μg/dose to about 100 μg/dose, or about 25 μg/dose to about 50 μg/dose, or about 25 μg/dose to about 60 μg/dose and wherein the inhalation mixture is substantially free of pharmaceutically active agents other than the corticosteroid. In some embodiments, the corticosteroid is budesonide. In other embodiments, the corticosteroid is budesonide wherein the budesonide is either an individual diastereomer or a mixture of the two diastereomers administered individually or together for a therapeutic effect.

In still other embodiments, the systems and methods described herein provide the delivery of an aqueous inhalation mixture comprising a corticosteroid, a solvent, and a solubility enhancer wherein the delivery of the corticosteroid provides enhanced pharmacokinetic profiles of the corticosteroid as compared to the delivery of the corticosteroid by conventional inhalable suspension-based therapies. In some embodiments, the corticosteroid is budesonide. In other embodiments, the corticosteroid is budesonide wherein the budesonide is either an individual diastereomer or a mixture of the two diastereomers administered individually or together for a therapeutic effect.

The corticosteroids that are useful in the present invention include, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In some embodiments, the corticosteroid is budesonide. In other embodiments, the corticosteroid is budesonide wherein the budesonide is either an individual diastereomer or a mixture of the two diastereomers administered individually or together for a therapeutic effect.

In certain other embodiments, the corticosteroid is selected group of corticosteroids in the foregoing paragraph not including the betamethasone.

In another embodiment, the present invention can provide a method for reducing the risk of side effects associated with corticosteroid inhalation therapy whereby a lower nominal dosage of the corticosteroid is required to achieve a therapeutic effect as compared to conventional inhalable corticosteroid therapies. In one embodiment, the risk of side effects is lowered compared to conventional inhalable corticosteroid therapies wherein the corticosteroid is administered at nominal dosage of less than about 250 μg/dose. In another embodiment, the risk of side effects is lowered compared to conventional inhalable corticosteroid therapies wherein the corticosteroid is administered at nominal dosage of less than about 240 μg/dose. In still another embodiment, the risk of side effects is lowered compared to conventional inhalable corticosteroid therapies wherein the corticosteroid is administered at nominal dosage of less than about 200 μg/dose. In yet another embodiment, the risk of side effects is lowered compared to conventional inhalable corticosteroid therapies wherein the corticosteroid is administered at nominal dosage of less than about 150 μg/dose. In yet another embodiment, the risk of side effects is lowered compared to conventional inhalable corticosteroid therapies wherein the corticosteroid is administered at nominal dosage of less than about 125 μg/dose. In still yet another embodiment, the risk of side effects is lowered compared to conventional inhalable corticosteroid therapies wherein the corticosteroid is administered at nominal dosage of less than about 100 μg/dose. In yet still another embodiment, the risk of side effects is lowered compared to conventional inhalable corticosteroid therapies wherein the corticosteroid is administered at nominal dosage of less than about 60 μg/dose. In still yet another embodiment, the risk of side effects is lowered compared to conventional inhalable corticosteroid therapies wherein the corticosteroid is administered at nominal dosage of less than about 50 μg/dose. In some embodiments, the corticosteroid is budesonide administered at nominal dosage of less than about 250 μg/dose. In other embodiments, the corticosteroid is budesonide administered at nominal dosage of less than about 125 μg/dose. In still other embodiments, the corticosteroid is budesonide administered at nominal dosage of about 120 μg/dose. In yet other embodiments, the corticosteroid is budesonide administered at nominal dosage of about 60 μg/dose. In still yet other embodiments, the corticosteroid is budesonide administered at nominal dosage of about 40 μg/dose.

The systems and methods described herein provide the delivery of an aqueous inhalation mixture comprising a corticosteroid and a solubility enhancer. In some embodiments, suitable aqueous inhalation mixtures comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

The systems and methods described herein provide the delivery of an aqueous inhalation mixture comprising a corticosteroid, a solvent and a solubility enhancer. In some embodiments, suitable aqueous inhalation mixtures comprising a corticosteroid include, but are not limited to, solutions, dispersions, nano-dispersions, emulsions, colloidal liquids, micelle or mixed micelle solutions, and liposomal liquids. In one embodiment, the aqueous inhalation mixture is a solution comprising a corticosteroid, such as budesonide, and a solubility enhancer. In another embodiment, the aqueous inhalation mixture is a mixed micelle solution comprising a corticosteroid, such as budesonide, and a solubility enhancer. In yet another embodiment, the aqueous inhalation mixture is a liposomal solution comprising a corticosteroid, such as budesonide, and a solubility enhancer.

In some embodiments of the invention, inhalable compositions comprising a corticosteroid do not include nano-dispersions and/or nano-suspensions. In other embodiments, inhalable compositions comprising a corticosteroid do not include micelle, mixed-micelle liquids or liposomal liquids. In still other embodiments, inhalable compositions comprising a corticosteroid do not include nano-dispersions and/or nano-suspensions, micelle, mixed-micelle liquids or liposomal liquids. In other embodiments, inhalable compositions, include but are not limited to, solutions, emulsions, and colloidal liquids. In one embodiment, the inhalable composition is a solution comprising a corticosteroid, such as budesonide, and a solubility enhancer. In another embodiment, the inhalable composition is an emulsion comprising a corticosteroid, such as budesonide, and a solubility enhancer.

In certain embodiments, the systems and methods described herein comprise a solvent. In certain embodiments, the solvent is selected from the group consisting of water, water/ethanol mixture, aqueous alcohol, propylene glycol, or aqueous organic solvent, or combinations thereof. In certain embodiments, the solvent comprises water. In preferred embodiments, the solvent is water.

In some embodiments of the systems and methods described herein, a corticosteroid-containing aqueous inhalation mixture is employed which further comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

Chemical agents acting as solubility enhancers suitable for use in the present invention include, but are not limited to, propylene glycol, non-ionic surfactants, phospholipids, cyclodextrins and derivatives thereof, and surface modifiers and/or stabilizers. In other embodiments, solubility enhancers refer to a formulation method which provides enhanced solubility without a chemical agent acting as the means to increase solubility, e.g. the use of super critical fluid production methods to generate nanoparticles for dispersion in a solvent.

Additional solubility enhancers are known in the art and are described in, e.g., U.S. Pat. Nos. 5,134,127, 5,145,684, 5,376,645, 6,241,969 and U.S. Pub. Appl. Nos. 2005/0244339 and 2005/0008707, each of which is specifically incorporated by reference herein. In addition, examples of suitable solubility enhancers are described below.

Suitable cyclodextrins and derivatives for use in the present invention are described in the art, for example, Challa et al., AAPS PharmSciTech 6(2): E329-E357 (2005), U.S. Pat. Nos. 5,134,127, 5,376,645, 5,874,418, each of which is specifically incorporated by reference herein. In some embodiments, suitable cyclodextrins or cyclodextrin derivatives for use in the present invention include, but are not limited to, α-cyclodextrins, β-cyclodextrins, -γ-cyclodextrins, SAE-CD derivatives (e.g., SBE-α-CD, SBE-β-CD, SBE1-β-CD, SBE4-β-CD, SBE7-β-CD (Captisol®), and SBE-γ-CD) (Cydex, Inc. Lenexa, Kans.), hydroxyethyl, hydroxypropyl (including 2- and 3-hydroxypropyl) and dihydroxypropyl ethers, their corresponding mixed ethers and further mixed ethers with methyl or ethyl groups, such as methylhydroxyethyl, ethyl-hydroxyethyl and ethyl-hydroxypropyl ethers of α-, β- and γ-cyclodextrin; and the maltosyl, glucosyl and maltotriosyl derivatives of α-, β- and -γ-cyclodextrin, which may contain one or more sugar residues, e.g. glucosyl or diglucosyl, maltosyl or dimaltosyl, as well as various mixtures thereof, e.g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Procedures for preparing such cyclodextrin derivatives are well-known, for example, from U.S. Pat. No. 5,024,998, and references incorporated by reference therein. Other cyclodextrins suitable for use in the present invention include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. Nos. 6,610,671, 6,479,467, 6,660,804, or 6,509,323, each of which is specifically incorporated by reference herein.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e.g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, UK, 1999) and New Trends in Cyclodextrins and Derivatives (Ed. Dominique Duchene, Editions de Sante, Paris, France, 1991).

Examples of non-ionic surfactants which appear to have a particularly good physiological compatibility for use in the present invention are tyloxapol, polysorbates including, but not limited to, polyoxyethylene (20) sorbitan monolaurate, polyoxyethylene (20) sorbitan monopalmitate, polyoxyethylene (20) sorbitan monostearate (available under the tradename Tweens 20-40-60, etc.), Polysorbate 80, Polyethylene glycol 400; sodium lauryl sulfate; sorbitan laurate, sorbitan palmitate, sorbitan stearate (available under the tradename Span 20-40-60,etc.), benzalkonium chloride, PPO-PEO block copolymers (Pluronics), Cremophor-EL, vitamin E-TPGS (e.g., d-alpha-tocopheryl-polyethyleneglycol-1000-succinate), Solutol-HS-15, oleic acid PEO esters, stearic acid PEO esters, Triton-X100, Nonidet P-40, and macrogol hydroxystearates such as macrogol-15-hydroxystearate.

In some embodiments, the non-ionic surfactants suitable for use in the present invention are formulated with the corticosteroid to form liposome preparations, micelles or mixed micelles. Methods for the preparations and characterization of liposomes and liposome preparations are known in the art. Often, multi-lamellar vesicles will form spontaneously when amphiphilic lipids are hydrated, whereas the formation of small uni-lamellar vesicles usually requires a process involving substantial energy input, such as ultrasonication or high pressure homogenization. Further methods for preparing and characterizing liposomes have been described, for example, by S. Vemuri et al. (Preparation and characterization of liposomes as therapeutic delivery systems: a review. Pharm Acta Helv. 1995, 70(2):95-111) and U.S. Pat. Nos. 5,019,394, 5,192,228, 5,882,679, 6,656,497 each of which is specifically incorporated by reference herein.

In some cases, for example, micelles or mixed micelles may be formed by the surfactants, in which poorly soluble active agents can be solubilized. In general, micelles are understood as substantially spherical structures formed by the spontaneous and dynamic association of amphiphilic molecules, such as surfactants. Mixed micelles are micelles composed of different types of amphiphilic molecules. Both micelles and mixed micelles should not be understood as solid particles, as their structure, properties and behavior are much different from solids. The amphiphilic molecules which form the micelles usually associate temporarily. In a micellar solution, there is a dynamic exchange of molecules between the micelle-forming amphiphile and monomolecularly dispersed amphiphiles which are also present in the solution. The position of the drug molecules which are solubilized in such micelles or mixed micelles depends on the structure of these molecules as well as the surfactants used. For example, it is to be assumed that particularly non-polar molecules are localized mainly inside the colloidal structures, whereas polar substances are more likely to be found on the surface. In one embodiment of a micellar or mixed micellar solution, the average size of the micelles may be less than about 200 nm (as measured by photon correlation spectroscopy), such as from about 10 mm to about 100 nm. Particularly preferred are micelles with average diameters of about 10 to about 50 mm. Methods of producing micelles and mixed micelles are known in the art and described in, for example, U.S. Pat. Nos. 5,747,066 and 6,906,042, each of which is specifically incorporated by reference herein.

Phospholipids are defined as amphiphile lipids which contain phosphorus. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols, etc. Lecithins are natural mixtures of various phospholipids which usually have a high proportion of phosphatidyl cholines. Depending on the source of a particular lecithin and its method of extraction and/or enrichment, these mixtures may also comprise significant amounts of sterols, fatty acids, triglycerides and other substances.

Additional phospholipids which are suitable for delivery by inhalation on account of their physiological properties comprise, in particular, phospholipid mixtures which are extracted in the form of lecithin from natural sources such as soja beans (soy beans) or chickens egg yolk, preferably in hydrogenated form and/or freed from lysolecithins, as well as purified, enriched or partially synthetically prepared phospholipids, preferably with saturated fatty acid esters. Of the phospholipid mixtures, lecithin is particularly preferred. The enriched or partially synthetically prepared medium- to long-chain zwitterionic phospholipids are mainly free of unsaturations in the acyl chains and free of lysolecithins and peroxides. Examples for enriched or pure compounds are dimyristoyl phosphatidyl choline (DMPC), distearoyl phosphatidyl choline (DSPC) and dipalmitoyl phosphatidyl choline (DPPC). Of these, DMPC is currently more preferred. Alternatively, phospholipids with oleyl residues and phosphatidyl glycerol without choline residue are suitable for some embodiments and applications of the invention.

In some embodiments, the non-ionic surfactants and phospholipids suitable for use in the present invention are formulated with the corticosteroid to form colloidal structures. Colloidal solutions are defined as mono-phasic systems wherein the colloidal material dispersed within the colloidal solution does not have the measurable physical properties usually associated with a solid material. Methods of producing colloidal dispersions are known in the art, for example as described in U.S. Pat. No. 6,653,319, which is specifically incorporated by reference herein.

Suitable surface modifiers for use in the present invention are described in the art, for example, U.S. Pat. Nos. 5,145,684, 5,510,118, 5,565,188, and 6,264,922, each of which is specifically incorporated by reference herein. Examples of surface modifiers and/or surface stabilizers suitable for use in the present invention include, but are not limited to, hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens®, e.g., Tween 20® and Tween 80® (ICI Specialty Chemicals)), polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide), poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)), Tetronic 1508® (T-1508) (BASF Wyandotte Corporation), Tritons X-200®, which is an alkyl aryl polyether sulfonate (Rohm and Haas), Crodestas F-100®, which is a mixture of sucrose stearate and sucrose distearate (Croda Inc.), p-isononylphenoxypoly-(glycidol), also known as Olin-10G® or Surfactant 10® (Olin Chemicals, Stamford, Conn.), Crodestas SL-40.RTM. (Croda, Inc.), and SA9OHCO, which is C₁₈H₃₇CH₂(—CON(CH₃)—CH₂(CHOH)₄(CH₂OH)₂ (Eastman Kodak Co.), decanoyl-N-methylglucamide, n-decyl-β-D-glucopyranoside, n-decyl-β-D-maltopyranoside, n-dodecyl β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-noyl-β-D-glucopyranoside, octanoyl-N-methylglucamide, n-octyl-β-D-glucopyranoside, octyl β-D-thioglucopyranoside, PEG-phospholipid, PEG-cholesterol, PEG-cholesterol derivative, PEG-vitamin A, PEG-vitamin E, lysozyme, random copolymers of vinyl pyrrolidone and vinyl acetate, and the like (e.g. hydroxypropyl methylcellulose, hydroxypropylcellulose, polyvinylpyrrolidone, copolymers of vinyl acetate, vinyl pyrrolidone, sodium lauryl sulfate and dioctyl sodium sulfosuccinate).

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅ dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈) dimethylbenzyl ammonium chloride, N-alkyl (C₁₄₋₁₈) dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkylamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336®), POLYQUAT 10®, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, Mirapol® and ALKAQUAT® (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinyl salt, and alkylimidazolium salt, and amine oxides, imide azolinium salts, protonated quaternary acrylamides, methylated quaternary polymers, such as poly[diallyl dimethylammonium chloride] and poly-[N-methyl vinyl pyridinium chloride], and cationic guar.

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

Furthermore, the processes for producing nanometer sized particles, including SCF, can permit selection of a desired morphology (e.g., amorphous, crystalline, resolved racemic) by appropriate adjustment of the conditions for particle formation during precipitation or condensation. As a consequence of selection of the desired particle form, extended release of the selected medicament can be achieved. These particle fabrication processes are used to obtain nanoparticulates that have high purity, low surface imperfections, low surface charges and low sedimentation rates. Such particle features inhibit particle cohesion, agglomeration and also prevent settling in liquid dispersions. Additionally, because processes such as SCF can separate isomers of certain medicaments, such separation could contribute to the medicament's enhanced activity, effectiveness as well as extreme dose reduction. In some instances, isomer separation also contributes to reduced side effects. In accordance with the present methods and systems, an aqueous inhalation mixture can be a composition fabricated into a powdered form by any process including SCF, spray drying, precipitation and volume exclusion, directly into a collection media, wherein the particulate compound is thus automatically generated into a dispersed formulation. In some embodiments, this formulation can be the final formulation.

In some embodiments of this invention, the solubility enhancer is a chemical agent selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain embodiments, the solubility enhancer is SBE7-β-CD (Captisol®).

Any known inhalation nebulizer is suitable for use in the presently described invention. Such nebulizers include, e.g., jet nebulizers, ultrasonic nebulizers, pulsating membrane nebulizers, nebulizers with a vibrating mesh or plate with multiple apertures, and nebulizers comprising a vibration generator and an aqueous chamber (e.g., Pari eFlow®). Commercially available air driven jet, ultrasonic or pulsating membrane nebulizers suitable for use in the present invention include the Aeroneb®, Aeroneb GO® (Aerogen, San Francisco, Calif.), Pari LC PLUS®, Pari Boy® N and Pari Duraneb® (PARI Respiratory Equipment, Inc., Monterey, Calif.), MicroAir® (Omron Healthcare, Inc., Vernon Hills, Ill.), Halolite® (Profile Therapeutics Inc., Boston, Mass.), Respimat® (Boehringer Ingelheim Ingelheim, Germany), Aerodose® (Aerogen, Inc., Mountain View, Calif.), Omron Elite® (Omron Healthcare, Inc., Vernon Hills, Ill.), Omron Microair® (Omron Healthcare, Inc., Vernon Hills, Ill.), Mabismist II® (Mabis Healthcare, Inc., Lake Forest, Ill.), Lumiscope® 6610, (The Lumiscope Company, Inc., East Brunswick, N.J.), Airsep Mystique®, (AirSep Corporation, Buffalo, N.Y.), Acorn-1 and Acorn-II (Vital Signs, Inc., Totowa, N.J.), Aquatower® (Medical Industries America, Adel, Iowa), Ava-Neb® (Hudson Respiratory Care Incorporated, Temecula, Calif.), Cirrus® (Intersurgical Incorporated, Liverpool, N.Y.), Dart® (Professional Medical Products, Greenwood, S.C.), Devilbiss® Pulmo Aide (DeVilbiss Corp. Somerset, Pa.), Downdraft® (Marquest, Englewood, Colo.), Fan Jet® (Marquest, Englewood, Colo.), MB-5 (Mefar, Bovezzo, Italy), Misty Neb® (Baxter, Valencia, Calif.), Salter 8900 (Salter Labs, Arvin, Calif.), Sidestream® (Medic-Aid, Sussex, UK), Updraft-II® (Hudson Respiratory Care; Temecula, Calif.), Whisper Jet® (Marquest Medical Products, Englewood, Colo.), Aiolos® (Aiolos Medicnnsk Teknik, Karlstad, Sweden), Inspiron® (Intertech Resources, Inc., Bannockburn, Ill.), Optimist® (Unomedical Inc., McAllen, Texas), Prodomo®, Spira® (Respiratory Care Center, Hameenlinna, Finland), AERx (Aradigm Corporation, Hayward, Calif.), Sonik® LDI Nebulizer (Evit Labs, Sacramento, Calif.), and Swirler W Radioaerosol System (AMICI, Inc., Spring City, Pa.).

Any of these and other known nebulizers can be used to deliver the aqueous inhalation mixtures described in the present invention. In some embodiments, the nebulizers are available from, e.g., Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others.

Other nebulizers suitable for use in the methods and systems describe herein include, but are not limited to, jet nebulizers (optionally sold with compressors), ultrasonic nebulizers, and others. Exemplary jet nebulizers for use herein include Pari LC plus/ProNeb, Pari LC plus/ProNeb Turbo, Pari LCPlus/Dura Neb 1000 & 2000 Pari LC plus/Walkhaler, Pari LC plus/Pari Master, Pari LC star, Omron CompAir XL Portable Nebulizer System (NE-C18 and JetAir Disposable nebulizer), Omron compare Elite Compressor Nebulizer System (NE-C21 and Elite Air Reusable Nebulizer, Pari LC Plus or Pari LC Star nebulizer with Proneb Ultra compressor, Pulomo-aide, Pulmo-aide LT, Pulmo-aide traveler, Invacare Passport, Inspiration Healthdyne 626, Pulmo-Neb Traveler, DeVilbiss 646, Whisper Jet, AcornII, Misty-Neb, Allied aerosol, Schuco Home Care, Lexan Plasic Pocet Neb, Side Stream Hand Held Neb, Mobil Mist, Up-Draft, Up-DraftII, T Up-Draft, ISO-NEB, Ava-Neb, Micro Mist, and PulmoMate.

Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer, and Mabismist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb Portable Nebulizer System, Aerodose Inhaler, and AeroEclipse Breath Actuated Nebulizer. Exemplary nebulizers comprising a vibrating mesh or plate with multiple apertures are described by R. Dhand, (New Nebuliser Technology—Aerosol Generation by Using a Vibrating Mesh or Plate with Multiple Apertures, Long-Term Healthcare Strategies 2003, (July 2003), p. 1-4) and Respiratory Care, 47: 1406-1416 (2002), the entire disclosure of each of which is hereby incorporated by reference.

Additional nebulizers suitable for use in the presently described invention include nebulizers comprising a vibration generator and an aqueous chamber. Such nebulizers are sold commercially as, e.g., Pari eFlow®, and are described in U.S. Pat. Nos. 6,962,151, 5,518,179, 5,261,601, and 5,152,456, each of which is specifically incorporated by reference herein.

The parameters used in nebulization, such as flow rate, mesh membrane size, aerosol inhalation chamber size, mask size and materials, valves, and power source may be varied in accordance with the principles of the present invention to maximize their use with different types and aqueous inhalation mixtures or different types of corticosteroids.

In addition to the above cited nebulizers, atomizers are also suitable for the systems and methods described herein for the delivery of an aqueous inhalation solution comprising a corticosteroid and a solubility enhancer. Atomizers are known in the art and are described in, for example, U.S. Pat. Nos. 5,954,047, 6,026,808, 6,095,141 and 6,527,151, each of which is specifically incorporated by reference.

In certain preferred embodiments, the methods and systems described herein comprise a nebulizer selected from the group consisting of a jet nebulizer, an ultrasonic nebulizer, a pulsating membrane nebulizer, a nebulizer comprising a vibrating mesh or plate with multiple apertures, or a nebulizer comprising a vibration generator and an aqueous chamber. In some embodiments of this invention, the nebulizer is selected from the group consisting of Pari LC Jet Plus, Intertech, Baxter Misty-Neb, Hudson T-Updraft II, Hudson Ava-Neb, Aiolos, Pari LC Jet, DeVilbiss Pulmo-Neb, Hudson Iso-Neb (B), Hudson T-Updraft Neb-U-Mist, Pari-Jet 1460, and AeroTech with T-piece. In certain other embodiments, the nebulizer is a Pari eFlow nebulizer.

In other aspects of the invention, the methods and systems described herein can deliver an aqueous inhalable mixture comprising a corticosteroid, e.g. budesonide, to a subject in therapeutically effective amount for the treatment of a subject that has had or is anticipating a bronchoconstrictive disorder selected from the group consisting of asthma, pediatric asthma, bronchial asthma, allergic asthma, intrinsic asthma, chronic obstructive pulmonary disease (COPD), chronic bronchitis, emphysema, or a combination of any of the above.

In still other aspects of the invention, the methods and systems described herein comprise an aqueous inhalation mixture comprising a corticosteroid administered according to the methods and systems described herein not more than twice a day (b.i.d). In still another aspect, the aqueous inhalation mixture comprises a corticosteroid, such as budesonide, wherein the aqueous inhalation mixture is administered according to the methods and systems described herein twice a day. In yet another aspect, the aqueous inhalation mixture comprises a corticosteroid, such as budesonide, wherein the aqueous inhalation mixture is administered according to the methods and systems described herein not more than once a day. In still yet another aspect, the aqueous inhalation mixture comprises a corticosteroid, such as budesonide, wherein the aqueous inhalation mixture is administered according to the methods and systems described herein once a day. In still another embodiment, the aqueous inhalation mixture comprises a corticosteroid, such as budesonide, wherein the aqueous inhalation mixture is administered according to the methods and systems described herein not more than once a day in the evening.

In other embodiments, the methods and systems described herein can further include administering aqueous inhalation mixtures comprising a corticosteroid in combination with one or more active agents. In some embodiments, the corticosteroid, e.g., budesonide, can be administered in combination with one or more other drugs one or more active agents selected from the group consisting of: (a) a B2-adrenoreceptor agonist; (b) a dopamine (D2) receptor agonist; (c) a prophylactic therapeutic, such as a steroid; (d) a topical anesthetic; or (e) an anti-cholinergic agent; either simultaneously with, prior to or subsequent to the inhalable composition provided herein.

In another aspect of the present invention, the systems and methods described herein can provide a more efficient dose by weight of a corticosteroid. In some embodiments, a significantly greater amount of the corticosteroid dose by weight provided in an aqueous inhalation mixture according to the systems and methods described herein can be absorbed into the bloodstream of a patient as compared to conventional inhalable corticosteroid therapies. In certain embodiments, the systems and methods described herein can deliver an inhalation mixture comprising budesonide and a solubility enhancer wherein greater than greater than about 55%; or greater than about 50%; or greater than about 45%; or greater than about 40%; or greater than about 35%; or greater than about 30%; or greater than about 25%; or greater than about 20% of the budesonide dosed by weight is absorbed into the bloodstream.

In still other embodiments, the methods and systems of the present invention can deliver a therapeutically effective amount of a corticosteroid in a significantly shorter period of time than conventional inhalable corticosteroid therapies. For example, the nebulization time for Pulmicort® Respules administered by a Pari LC Plus jet nebulizer takes at least 5 minutes to 8 minutes, and in some cases in excess of 10 minutes. By contrast, the methods and systems of the present invention can deliver a therapeutically effective amount of a corticosteroid, such as a budesonide, over a delivery time of less than about 5 minutes to less than about 1.5 minutes. In some embodiments, the delivery time can be about 5 minutes. In other embodiments, the delivery time can be less than about 5 minutes. In certain embodiments, the delivery time can be about 4.5 minutes. In certain other embodiments, the delivery time can be less than about 4.5 minutes. In still other embodiments, the delivery time can be about 4 minutes. In yet other embodiments, the delivery time can be less than about 4 minutes. In still yet other embodiments, the delivery time can be about 3.5 minutes. In other embodiments, the delivery time can be less than about 3.5 minutes. In yet still other embodiments, the delivery time can be about 3 minutes. In other embodiments, the delivery time can be less than about 3 minutes. In certain embodiments, the delivery time can be about 2.5 minutes. In other certain embodiments, the delivery time can be less than about 2.5 minutes. In still other embodiments, the delivery time can be about 2 minutes. In yet still other embodiments, the delivery time can be less than about 2 minutes. In a preferred embodiment, the delivery time can be about 1.5 minutes. In a more preferred embodiment, the delivery time can be less than about 1.5 minutes.

In other embodiments, the methods and systems of the present invention can deliver substantially all of the nominal dosage of a corticosteroid in a significantly shorter period of time than conventional inhalable corticosteroid therapies. For example, the nebulization time for Pulmicort® Respules administered by a Pari LC Plus jet nebulizer takes at least 5 minutes to 8 minutes, and in some cases in excess of 10 minutes. By contrast, the methods and systems of the present invention can deliver an nominal dosage of a corticosteroid, such as a budesonide, over a delivery time of less than about 5 minutes to less than about 1.5 minutes. In some embodiments, substantially all of the nominal dosage can be delivered in about 5 minutes. In other embodiments, substantially all of the nominal dosage can be delivered in less than about 5 minutes. In certain embodiments, substantially all of the nominal dosage can be delivered in about 4.5 minutes. In certain other embodiments, substantially all of the nominal dosage can be delivered in than about 4.5 minutes. In still other embodiments, substantially all of the nominal dosage can be delivered in about 4 minutes. In yet other embodiments, substantially all of the nominal dosage can be delivered in less than about 4 minutes. In still yet other embodiments, substantially all of the nominal dosage can be delivered in about 3.5 minutes. In other embodiments, substantially all of the nominal dosage can be delivered than about 3.5 minutes. In yet still other embodiments, substantially all of the nominal dosage can be delivered in about 3 minutes. In other embodiments, substantially all of the nominal dosage can be delivered in less than about 3 minutes. In certain embodiments, substantially all of the nominal dosage can be delivered in about 2.5 minutes. In other certain embodiments, substantially all of the nominal dosage can be delivered in less than about 2.5 minutes. In still other embodiments, substantially all of the nominal dosage can be delivered about 2 minutes. In yet still other embodiments, substantially all of the nominal dosage can be delivered in less than about 2 minutes. In a preferred embodiment, substantially all of the nominal dosage can be delivered in about 1.5 minutes. In a more preferred embodiment, substantially all of the nominal dosage can be delivered in less than about 1.5 minutes.

In certain other embodiments, the methods and systems of the present invention can deliver a therapeutically effective amount of a corticosteroid in a unit dose comprising a smaller volume than conventional inhalable corticosteroid therapies. For example, the systems and methods of the present invention can deliver a therapeutically effective amount of a corticosteroid, such as a budesonide, wherein the volume of the aqueous inhalation mixture is from about 0.5 ml to less than 5 mls. In some embodiments, the volume of the aqueous inhalation mixture can about 3.5 mls. In other embodiments, the volume of the aqueous inhalation mixture can be about 3.0 mls. In still other embodiments, the volume of the aqueous inhalation mixture can be about 2.5 mls. In yet other embodiments, the volume of the aqueous inhalation mixture can be about 2.0 mls. In certain embodiments, the volume of the aqueous inhalation mixture can be about 1.5 mls. In other certain embodiments, the volume of the aqueous inhalation mixture can be about 1.0 mls. In a preferred embodiment, the volume of the aqueous inhalation mixture can be about 0.5 mls.

It is to be understood that the aspects of the methods and systems described herein can comprise one or more of any or all of the advantages provided by the present invention, and additional embodiments are within the scope of the invention. For example, the methods and systems described herein can provide an aqueous inhalation mixture comprising a corticosteroid in a nominal dosage of about 60 μg/dose and a solubility enhancer with a volume of the inhalation mixture of about 0.5 mls and an inhalable nebulizer, wherein the delivery of the aqueous mixture comprising the corticosteroid by the nebulizer is less than about 2 minutes, and wherein the delivery of the aqueous inhalation mixture comprising the corticosteroid by the nebulizer results in an enhanced pharmacokinetic profile of the corticosteroid such that the Cₘₐₓ is equal to the Cₘₐₓ of a inhalable suspension comprising a corticosteroid when the aqueous inhalation mixture comprising the corticosteroid at a nominal dosage of less than about 100 μg/dose is about 60% of the nominal dosage of the inhalable suspension comprising a corticosteroid. The above embodiment is merely an example of one embodiment of the invention that incorporates numerous aspects or variations of the invention, and in no way is intended to limit the scope of the invention.

A. Delivery of a Corticosteroid Displaying an Enhanced Pharmacokinetic Profile

The present invention can also provide a method or system for the treatment or prophylaxis of a bronchoconstrictive disorder in a patient comprising providing an aqueous inhalation mixture comprising a nominal dosage of a corticosteroid and a solubility enhancer and delivering the aqueous inhalation mixture via an inhalation nebulizer. In these embodiments, the methods can provide the delivery of the corticosteroid displaying an enhanced pharmacokinetic profile as compared to a suspension-based corticosteroid formulation, administered under the same conditions.

In certain embodiments, the methods and systems of the present invention, in certain embodiments, can provide for treatment or prophylaxis of a bronchoconstrictive disorder in a patient in need thereof comprising (a) providing an aqueous inhalation mixture comprising a nominal dosage of a corticosteroid and a solubility enhancer and (b) delivering the aqueous inhalation mixture comprising the corticosteroid with the nebulizer, whereby the methods and systems provide at least a two-fold enhanced pharmacokinetic profile of the aqueous inhalation mixture comprising a nominal dosage of a corticosteroid as compared to the pharmacokinetic profile of an inhalable suspension comprising a nominal dosage of a corticosteroid, e.g. Pulmicort Respules®, administered under the same conditions. In some embodiments, the inhalable aqueous mixtures can display substantially equivalent bioavailability for the corticosteroid as compared to conventional inhalable suspensions comprising a corticosteroid, while using significantly lower nominal dosages. In other embodiments, the inhalable aqueous mixtures can display increased bioavailability of the corticosteroid as compared to conventional inhalable suspensions comprising a corticosteroid when delivered at the same nominal dosage. In certain embodiments, the ratio of the nominal dosage of the corticosteroid in the aqueous inhalation mixture to the nominal dosage the corticosteroid in the inhalable suspension is from about 0.01:1 to about 1:100.

In other embodiments, methods and systems of the present invention can provide for treatment or prophylaxis of a bronchoconstrictive disorder in a patient in need thereof comprising (a) providing an aqueous inhalation mixture comprising a nominal dosage of a single corticosteroid and a solubility enhancer and (b) delivering the aqueous inhalation mixture comprising the corticosteroid with the nebulizer, whereby the methods and systems provide at least a two-fold enhanced pharmacokinetic profile of the aqueous inhalation mixture comprising a nominal dosage of a corticosteroid as compared to the pharmacokinetic profile of an inhalable suspension comprising a nominal dosage of a corticosteroid, e.g. Pulmicort Respules®, administered under the same conditions wherein the inhalation mixture is substantially free of pharmaceutically active agents other than the corticosteroid. In some embodiments, the inhalable aqueous mixtures can display substantially equivalent bioavailability for the corticosteroid as compared to conventional inhalable suspensions comprising a corticosteroid, while using significantly lower nominal dosages. In other embodiments, the inhalable aqueous mixtures can display increased bioavailability of the corticosteroid as compared to conventional inhalable suspensions comprising a corticosteroid when delivered at the same nominal dosage. In certain embodiments, the ratio of the nominal dosage of the corticosteroid in the aqueous inhalation mixture to the nominal dosage the corticosteroid in the inhalable suspension is from about 0.01:1 to about 1:100.

In certain embodiments, the aqueous inhalation mixture comprising a corticosteroid delivered by the methods described herein can have a Cₘₐₓ greater than the Cₘₐₓ of a suspension-based corticosteroid formulation administered at the same nominal dosage under the same conditions. In other embodiments, the aqueous inhalation mixture comprising a corticosteroid delivered by the methods described herein can have an AUC₍ₗₐₛₜ₎ greater than the AUC₍ₗₐₛₜ₎ of a suspension-based corticosteroid formulation administered at the same nominal dosage under the same conditions. In still other embodiments, the aqueous inhalation mixture comprising a corticosteroid delivered by the method described herein can have an AUC_{(0-∞)} greater than the AUC_{(0-∞)} of a suspension-based corticosteroid formulation administered at the same nominal dosage under the same conditions. In yet other embodiments, the aqueous inhalation mixture comprising a corticosteroid delivered by the method described herein can have a Tₘₐₓ less than the Tₘₐₓ of a suspension-based corticosteroid formulation administered under the same conditions.

In certain other embodiments, the aqueous inhalation mixture comprising a corticosteroid delivered by the methods described herein can have a Cₘₐₓ equivalent to the Cₘₐₓ of a suspension-based corticosteroid formulation wherein the nominal dosage of the aqueous inhalation mixture is lower than the nominal dosage of the suspension-based corticosteroid formulation administered under the same conditions. In other embodiments, the aqueous inhalation mixture comprising a corticosteroid delivered by the methods described herein can have an AUC₍ₗₐₛₜ₎ equivalent to the AUC₍ₗₐₛₜ₎ of a suspension-based corticosteroid formulation wherein the nominal dosage of the aqueous inhalation mixture is lower than the nominal dosage of the suspension-based corticosteroid formulation administered under the same conditions. In still other embodiments, the aqueous inhalation mixture comprising a corticosteroid delivered by the method described herein can have an AUC_{(0-∞)} equivalent to the AUC_{(0-∞)} of a suspension-based corticosteroid formulation wherein the nominal dosage of the aqueous inhalation mixture is lower than the nominal dosage of the suspension-based corticosteroid formulation administered under the same conditions. In yet other embodiments, the aqueous inhalation mixture comprising a corticosteroid delivered by the method described herein can have a Tₘₐₓ less than the Tₘₐₓ of a suspension-based corticosteroid formulation wherein the nominal dosage of the aqueous inhalation mixture is lower than the nominal dosage of the suspension-based corticosteroid formulation administered under the same conditions.

As previously stated, increased exposure to elevated blood plasma levels of a corticosteroid can result in undesirable side effects. Thus, lower doses of a corticosteroid which can achieve the same or better therapeutic effects as those observed with larger doses of conventional inhalable corticosteroid therapies are desired. Such lower doses can be realized with the methods and system described herein as a result of the greater bioavailability of the corticosteroid as compared to conventional inhalable suspensions comprising a corticosteroid. The methods and system described herein can deliver a corticosteroid with an enhanced pharmacokinetic profile of the aqueous inhalation mixture comprising a nominal dosage of a corticosteroid as compared to the pharmacokinetic profile of an inhalable suspension comprising a nominal dosage of a corticosteroid, e.g. Pulmicort Respules®, administered under the same conditions in a range of between at least about 1.5 fold (150%) to about 10 fold (1000%) the specified therapeutic parameter (e.g., AUC_{(0-∞)}) to provide an enhanced pharmacokinetic profile. In certain embodiments, the ratio of the nominal dosage of the corticosteroid in the aqueous inhalation mixture to the nominal dosage the corticosteroid in the inhalable suspension is from about 0.01:1 to about 1:100.

In other embodiments, the methods and system described herein can deliver an aqueous inhalation mixture comprising a nominal dosage of a corticosteroid with an enhanced pharmacokinetic profile comprising an equivalent bioavailability (e.g., equivalent AUC_{(0-∞)}) compared to conventional inhalable suspensions comprising a nominal dosage of a corticosteroid wherein the nominal dosage of the corticosteroid in the aqueous inhalation mixtures is in a range of between at least about 1:1.5 to about 1:10 the nominal dose of the corticosteroid in conventional inhalable suspension to provide an enhanced pharmacokinetic profile (1.5 fold to about 10 fold enhanced pharmacokinetic profile).

In certain embodiments, the methods and systems of the present invention can provide for treatment or prophylaxis of a bronchoconstrictive disorder in a patient in need thereof comprising (a) providing an aqueous inhalation mixture comprising a nominal dosage of a corticosteroid and a solubility enhancer and (b) delivering the aqueous inhalation mixture comprising the corticosteroid with an inhalation nebulizer, whereby the methods and systems deliver about a 1.5 fold (150%) to about a 10 fold (1000%) enhanced pharmacokinetic profile of the aqueous inhalation mixture comprising the nominal dosage of the corticosteroid as compared to the pharmacokinetic profile of an inhalable suspension comprising a nominal dosage of a corticosteroid administered under the same conditions. In other embodiments, the inhalation mixture comprises a single corticosteroid and is substantially free of any other pharmaceutically active agent other than the corticosteroid. In one embodiment, the aqueous inhalation mixture comprising a nominal dosage of a corticosteroid administered by the systems and method described herein can deliver about a 1.5 fold (150%) and about a 9 fold (900%) an enhanced pharmacokinetic profile compared to conventional inhalable suspensions comprising a nominal dosage of a corticosteroid administered under the same conditions. In another embodiment, the aqueous inhalation mixture comprising nominal dosage of a corticosteroid administered by the systems and method described herein can deliver about a 1.5 fold (150%) and about a 8 fold (800%) have an enhanced pharmacokinetic profile compared to conventional inhalable suspensions comprising a nominal dosage of a corticosteroid administered under the same conditions. In yet another embodiment, the aqueous inhalation mixture comprising a nominal dosage of a corticosteroid administered by the systems and method described herein can have an enhanced pharmacokinetic profile compared to conventional inhalable suspensions comprising nominal dosage of a corticosteroid in a range of between about a 1.5 fold (150%) and about a 7 fold (700%) enhanced pharmacokinetic profile when administered under the same conditions. In still another embodiment, the aqueous inhalation mixture comprising a nominal dosage of a corticosteroid administered by the systems and method described herein can have an enhanced pharmacokinetic profile compared to conventional inhalable suspensions comprising a nominal dosage of corticosteroid in a range of between about a 1.5 fold (150%) and about a 6 fold (600%) enhanced pharmacokinetic profile when administered under the same conditions. In one embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid in a range of between about a 1.5 fold (150%) and about a 5 fold (500%) enhanced pharmacokinetic profile when administered under the same conditions. In yet another embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid in a range of between about a 1.5 fold (150%) and about a 4 fold (400%) enhanced pharmacokinetic profile when administered under the same conditions. In still another embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid in a range of between about a 1.5 fold (150%) and about a 3 fold (300%) enhanced pharmacokinetic profile when administered under the same conditions. In yet still another embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid in a range of between about a 1.5 fold (150%) and about a 2 fold (200%) enhanced pharmacokinetic profile when administered under the same conditions. In one embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid of about 2 fold (200%) when administered under the same conditions. In another embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid of about 3 fold (300%) when administered under the same conditions. In yet another embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid of about 4 fold (400%) when administered under the same conditions. In still another embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid of about 5 fold (500%) when administered under the same conditions. In yet still another embodiment, the aqueous inhalation mixture comprising a nominal dosage of a corticosteroid administered by the systems and method described herein can have an enhanced pharmacokinetic profile compared to conventional inhalable suspensions comprising a nominal dosage of a corticosteroid of about 6 fold (600%) when administered under the same conditions.

In certain other embodiments, an aqueous inhalation mixture comprising a nominal dosage of budesonide and a solubility enhancer is delivered by the systems and method described herein and has an enhanced pharmacokinetic profile compared to conventional inhalable suspensions comprising a nominal dosage of budesonide in a range of between at least about a 2 fold (200%) and about a 6 fold (600%) enhanced pharmacokinetic profile when administered under the same conditions. In one embodiment, the aqueous inhalation mixture comprising a nominal dosage of budesonide administered by the systems and method described herein can have an enhanced pharmacokinetic profile compared to conventional inhalable suspensions comprising a nominal dosage of budesonide of about 2 fold (200%) when administered under the same conditions. In another embodiment, the aqueous inhalation mixture comprising a nominal dosage of budesonide administered by the systems and method described herein can have an enhanced pharmacokinetic profile compared to conventional inhalable suspensions comprising a nominal dosage of budesonide of about 3 fold (300%) when administered under the same conditions. In yet another embodiment, the aqueous inhalation mixture comprising a nominal dosage of budesonide administered by the systems and method described herein can have an enhanced pharmacokinetic profile compared to conventional inhalable suspensions comprising a nominal dosage of budesonide of about 4 fold (400%) when administered under the same conditions. In still another embodiment, the aqueous inhalation mixture comprising a nominal dosage of budesonide administered by the systems and method described herein can have an enhanced pharmacokinetic profile compared to conventional inhalable suspensions comprising a nominal dosage of budesonide of about 5 fold (500%) when administered under the same conditions. In yet still another embodiment, the aqueous inhalation mixture comprising a nominal dosage of budesonide administered by the systems and method described herein can have an enhanced pharmacokinetic profile compared to conventional inhalable suspensions comprising a nominal dosage of budesonide of about 6 fold (600%) when administered under the same conditions.

Due to the enhanced pharmacokinetic profiles of corticosteroids provided by the methods and systems described herein, the present methods and systems for the treatment or prophylaxis of a bronchoconstrictive disorder in a patient in need thereof can deliver an aqueous inhalable mixture comprising a corticosteroid with the equivalent bioavailability as a inhalable suspension comprising of a corticosteroid wherein the aqueous inhalable mixture comprising a corticosteroid has a nominal dosage in a range of between about 1:1.5 to about 1:10 the nominal dosage of the inhalable suspension comprising a corticosteroid. In one embodiment, the methods and systems of the present invention can provide for treatment or prophylaxis of a bronchoconstrictive disorder in a patient in need thereof comprising (1) providing an aqueous inhalation mixture comprising a corticosteroid and a solubility enhancer and (2) delivering the aqueous inhalation mixture with a inhalation nebulizer, wherein the delivery of the aqueous mixture comprising the corticosteroid by the nebulizer can result in equivalent bioavailability of the corticosteroid as compared to conventional inhalable suspensions comprising a corticosteroid wherein the nominal dosage of the aqueous inhalation mixture comprising a corticosteroid is about 1:1.5 to about 1:10 the nominal dosage of the inhalable suspension comprising a corticosteroid. In another embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 to about 1:9 the nominal dosage of the inhalable suspension comprising a corticosteroid. In still another embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 to about 1:8 the nominal dosage of the inhalable suspension comprising a corticosteroid. In yet another embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 to about 1:7 the nominal dosage of the inhalable suspension comprising a corticosteroid. In still another embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 to about 1:6 the nominal dosage of the inhalable suspension comprising a corticosteroid. In one embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 and about 1:5 the nominal dosage of the inhalable suspension comprising a corticosteroid. In yet another embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 to about 1:4 the nominal dosage of the inhalable suspension comprising a corticosteroid. In still another embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 to about 1:3 the nominal dosage of the inhalable suspension comprising a corticosteroid. In yet still another embodiment, the aqueous inhalation mixture comprising a corticosteroid administered by the systems and method described herein can have an enhanced pharmacokinetic profile compared to conventional inhalable suspensions comprising a corticosteroid wherein the nominal dosage of the aqueous inhalation mixture comprising a corticosteroid is about 1:1.5 and about 1:2 the nominal dosage of the inhalable suspension comprising a corticosteroid.

In other embodiments, an aqueous inhalation mixture comprising a corticosteroid and a solubility enhancer is delivered by the systems and method described herein and has an enhanced pharmacokinetic profile compared to conventional inhalable suspensions comprising a corticosteroid wherein the nominal dosage of the aqueous inhalation mixture comprising a corticosteroid is about 1:2 to about 1:10 the nominal dosage of the inhalable suspension comprising a corticosteroid. In still other embodiments, an aqueous inhalation mixture comprising a corticosteroid and a solubility enhancer is delivered by the systems and method described herein and has an enhanced pharmacokinetic profile compared to conventional inhalable suspensions comprising a corticosteroid wherein the nominal dosage of the aqueous inhalation mixture comprising a corticosteroid is about 1:2 to about 1:5 the nominal dosage of the inhalable suspension comprising a corticosteroid. In yet still other embodiments, an aqueous inhalation mixture comprising a corticosteroid and a solubility enhancer is delivered by the systems and method described herein and has an enhanced pharmacokinetic profile compared to conventional inhalable suspensions comprising a corticosteroid wherein the nominal dosage of the aqueous inhalation mixture comprising a corticosteroid is about 1:2 to about 1:4 the nominal dosage of the inhalable suspension comprising a corticosteroid. In still other embodiments, an aqueous inhalation mixture comprising a corticosteroid and a solubility enhancer is delivered by the systems and method described herein and has an enhanced pharmacokinetic profile compared to conventional inhalable suspensions comprising a corticosteroid wherein the nominal dosage of the aqueous inhalation mixture comprising a corticosteroid is about 1:4 the nominal dosage of the inhalable suspension comprising a corticosteroid.

In certain other embodiments, an aqueous inhalation mixture comprising budesonide and a solubility enhancer is delivered by the systems and method described herein and has an enhanced pharmacokinetic profile compared to conventional inhalable suspensions comprising budesonide wherein the nominal dosage of the aqueous inhalation mixture is about 1:2 to about 1:10 the nominal dosage of the inhalable suspension comprising budesonide. In still other embodiments, an aqueous inhalation mixture comprising budesonide and a solubility enhancer is delivered by the systems and method described herein and has an enhanced pharmacokinetic profile compared to conventional inhalable suspensions comprising budesonide wherein the nominal dosage of the aqueous inhalation mixture is about 1:2 to about 1:5 the nominal dosage of the inhalable suspension comprising budesonide. In yet still other embodiments, an aqueous inhalation mixture comprising budesonide and a solubility enhancer is delivered by the systems and method described herein and has an enhanced pharmacokinetic profile compared to conventional inhalable suspensions comprising budesonide wherein the nominal dosage of the aqueous inhalation mixture is about 1:2 to about 1:4 the nominal dosage of the inhalable suspension comprising budesonide. In still other embodiments, an aqueous inhalation mixture comprising budesonide and a solubility enhancer is delivered by the systems and method described herein and has an enhanced pharmacokinetic profile compared to conventional inhalable suspensions comprising budesonide wherein the nominal dosage of the aqueous inhalation mixture is about 1:4 the nominal dosage of the inhalable suspension comprising budesonide.

In certain embodiments, the methods and systems of the present invention can deliver a therapeutically effective amount of a corticosteroid in a unit dose comprising a smaller volume than conventional inhalable corticosteroid therapies. For example, the systems and methods of the present invention can deliver a therapeutically effective amount of a corticosteroid, such as a budesonide, wherein the volume of the aqueous inhalation mixture is from about 0.5 ml to less than 5 mls. In some embodiments, the volume of the aqueous inhalation mixture can about 3.5 mls. In other embodiments, the volume of the aqueous inhalation mixture can be about 3.0 mls. In still other embodiments, the volume of the aqueous inhalation mixture can be about 2.5 mls. In yet other embodiments, the volume of the aqueous inhalation mixture can be about 2.0 mls. In certain embodiments, the volume of the aqueous inhalation mixture can be about 1.5 mls. In other certain embodiments, the volume of the aqueous inhalation mixture can be about 1.0 mls. In a preferred embodiment, the volume of the aqueous inhalation mixture can be about 0.5 mls.

In still other embodiments, the methods and systems of the present invention can deliver a therapeutically effective amount of a corticosteroid in a significantly shorter period of time than conventional inhalable corticosteroid therapies. For example, the nebulization time for Pulmicort® Respules administered by a Pari LC Plus jet nebulizer takes at least 5 minutes to 8 minutes, and in some cases in excess of 10 minutes. By contrast, the methods and systems of the present invention can deliver a therapeutically effective amount of a corticosteroid, such as a budesonide, over a delivery time of less than about 5 minutes to less than about 1.5 minutes. In some embodiments, the delivery time can be about 5 minutes. In other embodiments, the delivery time can be less than about 5 minutes. In certain embodiments, the delivery time can be about 4.5 minutes. In certain other embodiments, the delivery time can be less than about 4.5 minutes. In still other embodiments, the delivery time can be about 4 minutes. In yet other embodiments, the delivery time can be less than about 4 minutes. In still yet other embodiments, the delivery time can be about 3.5 minutes. In other embodiments, the delivery time can be less than about 3.5 minutes. In yet still other embodiments, the delivery time can be about 3 minutes. In other embodiments, the delivery time can be less than about 3 minutes. In certain embodiments, the delivery time can be about 2.5 minutes. In other certain embodiments, the delivery time can be less than about 2.5 minutes. In still other embodiments, the delivery time can be about 2 minutes. In yet still other embodiments, the delivery time can be less than about 2 minutes. In a preferred embodiment, the delivery time can be about 1.5 minutes. In a more preferred embodiment, the delivery time can be less than about 1.5 minutes.

In other embodiments, the methods and systems of the present invention can deliver substantially all of the nominal dosage of a corticosteroid in a significantly shorter period of time than conventional inhalable corticosteroid therapies. For example, the nebulization time for Pulmicort® Respules administered by a Pari LC Plus jet nebulizer takes at least 5 minutes to 8 minutes, and in some cases in excess of 10 minutes. By contrast, the methods and systems of the present invention can deliver substantially all of the nominal dosage of a corticosteroid, such as a budesonide, over a delivery time of less than about 5 minutes to less than about 1.5 minutes. In some embodiments, substantially all of the nominal dosage can be delivered in about 5 minutes. In other embodiments, substantially all of the nominal dosage can be delivered in less than about 5 minutes. In certain embodiments, substantially all of the nominal dosage can be delivered in about 4.5 minutes. In certain other embodiments, substantially all of the nominal dosage can be delivered in than about 4.5 minutes. In still other embodiments, substantially all of the nominal dosage can be delivered in about 4 minutes. In yet other embodiments, substantially all of the nominal dosage can be delivered in less than about 4 minutes. In still yet other embodiments, substantially all of the nominal dosage can be delivered in about 3.5 minutes. In other embodiments, substantially all of the nominal dosage can be delivered than about 3.5 minutes. In yet still other embodiments, substantially all of the nominal dosage can be delivered in about 3 minutes. In other embodiments, substantially all of the nominal dosage can be delivered in less than about 3 minutes. In certain embodiments, substantially all of the nominal dosage can be delivered in about 2.5 minutes. In other certain embodiments, substantially all of the nominal dosage can be delivered in less than about 2.5 minutes. In still other embodiments, substantially all of the nominal dosage can be delivered about 2 minutes. In yet still other embodiments, substantially all of the nominal dosage can be delivered in less than about 2 minutes. In a preferred embodiment, substantially all of the nominal dosage can be delivered in about 1.5 minutes. In a more preferred embodiment, substantially all of the nominal dosage can be delivered in less than about 1.5 minutes.

B. Cₘₐₓ Blood Plasma Values

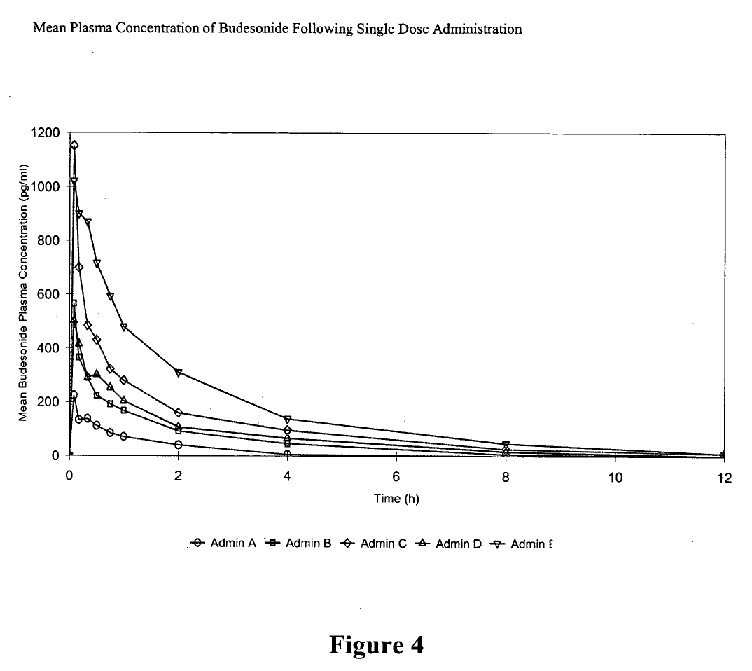

The methods and systems for the treatment or prophylaxis of a bronchoconstrictive disorder in a patient in need thereof described herein can deliver an inhalation mixture comprising corticosteroid to the subject in a manner wherein the active is delivered having an increased Cₘₐₓ blood plasma value of the corticosteroid as compared to conventional inhalable corticosteroid suspensions administered under the same conditions. In one example, conventional budesonide suspensions administered in a single dose using a Pari LC Plus® jet nebulizer in a 2.0 ml volume with an administration time of about 5 minutes display pharmacokinetic profiles such that the Cₘₐₓ blood plasma values ranges from about 556±193 (pg/ml) to about 1114±593 (pg/ml) with nominal dosages of 500 μg to 1000 μg, respectively. Using the systems and methods described herein, budesonide+SBE7-β-CD inhalation solutions having nominal dosages of 60 μg, 120 μg, and 240 μg delivered in a single dose using a Pari eFlow Inhaler® in a 0.5 ml volume with a delivery time of about 1.5 minutes had Cₘₐₓ blood plasma values of about 227±89 (pg/ml), about 578±238 (pg/ml), and 1195±811 (pg/ml), respectively. FIG. 4 provides a graphic representation of the data used to generate the aforementioned Cₘₐₓ blood plasma values.

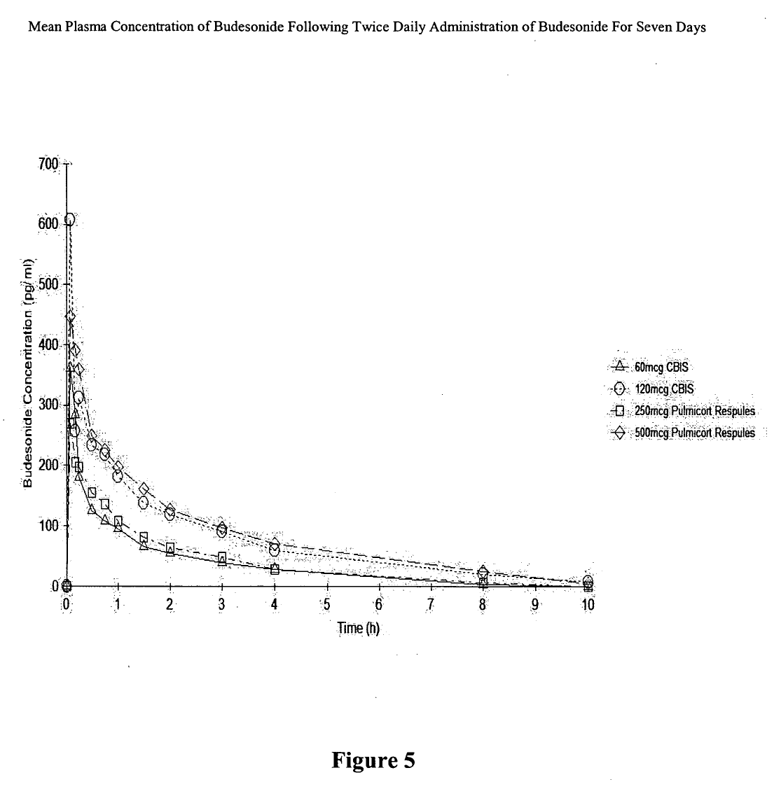

In a second example, conventional budesonide suspensions (Pulmicort Respules®) administered twice daily for seven days using a Pari LC Plus® jet nebulizer in a 2.0 ml volume with an administration time of about 4 minutes displayed pharmacokinetic profiles having mean Cₘₐₓ blood plasma values of 319.6±185 pg/ml and about 491.4±207 pg/ml with nominal dosages of 250 μg and 500 μg, respectively. The same 250 μg and 500 μg Pulmicort Respules® inhalation suspensions had geometric mean values for Cₘₐₓ blood plasma of 270.5 pg/ml and 451.6 pg/ml, respectively. Using the systems and methods described herein, a 60 μg CBIS inhalation solution delivered twice daily for seven days using a Pari eFlow Inhaler® in a 0.5 ml volume with a delivery time of about 1.5 minutes had a minimum Cₘₐₓ blood plasma value of about 186.4 pg/ml, a maximum Cₘₐₓ blood plasma value of about 779.4 pg/ml, and geometric mean Cₘₐₓ values of about 362.2 pg/ml. Likewise, a 120 μg CBIS inhalation solution delivered twice daily for seven days using a Pari eFlow Inhaler® in a 0.5 ml volume with a delivery time of about 1.5 minutes had a minimum Cₘₐₓ blood plasma value of about 169.8 pg/ml, a maximum Cₘₐₓ blood plasma value of about 1160.4 pg/ml, and geometric mean Cₘₐₓ values of about 516.9 pg/ml. FIG. 5 provides a graphic representation of the data upon which the aforementioned Cₘₐₓ blood plasma values were based.

Thus, the methods and systems for the treatment or prophylaxis of a bronchoconstrictive disorder in a patient in need thereof provide the delivery of an inhalation mixture comprising a nominal dosage of a corticosteroid having an enhanced pharmacokinetic profile as compared to conventional inhalable corticosteroid suspensions comprising a nominal dosage of a corticosteroid administered under the same conditions. More specifically, in certain embodiments, the systems and methods described herein provide at least about 1.5 fold to about 14 fold increase in Cₘₐₓ blood plasma values (as determined on an individual basis) for a corticosteroid normalized for dose of corticosteroid per microgram of corticosteroid administered, as compared to conventional inhalable corticosteroid therapies administered under the same conditions. In certain embodiments, the systems and methods described herein provide at least about 1.5 fold to about 13 fold, about 1.5 fold to about 12 fold, about 1.5 fold to about 10 fold, about 1.5 fold to about 8 fold, about 1.5 fold to about 7.5 fold, about 1.5 fold to about 7 fold, about 1.5 fold to about 6.5 fold, about 1.5 fold to about 6.25 fold, about 1.5 fold to about 6 fold, about 1.5 fold to about 5.75 fold, about 1.5 fold to about 5.5 fold, about 1.5 fold to about 5 fold, about 1.5 fold to about 4.75 fold, about 1.5 fold to about 4.5 fold, about 1.5 fold to about 4 fold increase in Cₘₐₓ blood plasma values (as determined on an individual basis) for a corticosteroid normalized for dose of corticosteroid per microgram of corticosteroid administered, as compared to conventional inhalable corticosteroid therapies administered under the same condition.

In other embodiments, the methods and systems for the treatment or prophylaxis of a bronchoconstrictive disorder in a patient in need thereof described herein provide at least a about 4 fold to about a 7 fold increase in Cₘₐₓ blood plasma values for a corticosteroid taken across a studied patient population, normalized for dose of corticosteroid per microgram of corticosteroid administered, as compared to conventional inhalable corticosteroid therapies administered under the same conditions, for example as determined using the geometric mean across a studied patient population. In certain embodiments, the systems and methods described herein provide at least about 4 fold to about 7 fold, about 4 fold to about 6.5 fold, about 4 fold to about 6.25 fold, about 4 fold to about 6 fold, about 4 fold to about 5.75 fold, about 4 fold to about 5.5 fold, about 4 fold to about 5 fold, about 5 fold to about 7 fold, about 5.5 fold to about 7 fold, about 6 fold to about 7 fold increase in Cₘₐₓ blood plasma values for a corticosteroid taken across a studied patient population, normalized for dose of corticosteroid per microgram of corticosteroid administered, as compared to conventional inhalable corticosteroid therapies administered under the same conditions.

In some embodiments, the Cₘₐₓ can be significantly greater than the Cₘₐₓ blood plasma values exhibited by conventional inhalable suspensions comprising a corticosteroid administered at the same nominal dosage under the same conditions. In certain embodiments, the Cₘₐₓ can be from about 1.5 fold (150%) to about 14 fold (1400%) the Cₘₐₓ blood plasma values exhibited by conventional inhalable suspensions comprising a corticosteroid at the same nominal dosage under the same conditions. In other embodiments, the Cₘₐₓ can be from about 1.5 fold (150%) to about 12 fold (1200%) the Cₘₐₓ blood plasma values exhibited by conventional inhalable suspensions comprising a corticosteroid at the same nominal dosage under the same conditions. In still other embodiments, the Cₘₐₓ can be from about 1.5 fold (150%) to about 10 fold (1000%) the Cₘₐₓ blood plasma values exhibited by conventional inhalable suspensions comprising a corticosteroid at the same nominal dosage under the same conditions. In one embodiment, the Cₘₐₓ can be at least about 12 fold (1200%) the Cₘₐₓ blood plasma values exhibited by conventional inhalable suspensions comprising a corticosteroid at the same nominal dosage under the same conditions. In certain other embodiments, the Cₘₐₓ can be at least about 1000% (10 fold) to about 1200% (12 fold), about 1100% (11 fold) to about 1200% (12 fold), or about 1150% (11.5 fold) to about 1200% (12 fold) greater than the Cₘₐₓ blood plasma values exhibited by conventional inhalable suspensions comprising a corticosteroid at the same nominal dosage under the same conditions. In other embodiments, the enhanced pharmacokinetic profile comprises a Cₘₐₓ for the aqueous inhalation mixture that is greater than the Cₘₐₓ of the inhalable suspension comprising a corticosteroid, administered at the same nominal dosage under the same conditions. In one embodiment, the Cₘₐₓ can be at least about 1000% (10 fold) greater than the Cₘₐₓ blood plasma values exhibited by conventional inhalable suspensions comprising a corticosteroid at the same nominal dosage under the same conditions. In certain other embodiments, the Cₘₐₓ can be at least about 900% (9 fold) to about 1000% (10 fold), about 925% (9.25 fold) to about 1000% (10 fold), or about 950% (9.5 fold) to about 1000% (10 fold) greater than the Cₘₐₓ blood plasma values exhibited by conventional inhalable suspensions comprising a corticosteroid at the same nominal dosage under the same conditions. In another embodiment, the Cₘₐₓ can be at least about 900% (9 fold) greater than the Cₘₐₓ blood plasma values exhibited by conventional inhalable suspensions comprising a corticosteroid at the same nominal dosage under the same conditions. In certain other embodiments, the Cₘₐₓ can be at least about 800% (8 fold) to about 900% (9 fold), about 825% (8.25 fold) to about 900% (9 fold), or about 850% (8.5 fold) to about 900% (9 fold) greater than the Cₘₐₓ blood plasma values exhibited by conventional inhalable suspensions comprising a corticosteroid at the same nominal dosage under the same conditions. In still another embodiment, the Cₘₐₓ can be at least about 800% (8 fold) greater than the Cₘₐₓ blood plasma values exhibited by conventional inhalable suspensions comprising a corticosteroid at the same nominal dosage under the same conditions. In certain other embodiments, the Cₘₐₓ can be at least about 700% (7 fold) to about 800% (8 fold), about 725% (7.25 fold) to about 800% (8 fold), or about 750% (7.5 fold) to about 800% (8 fold) greater than the Cₘₐₓ blood plasma values exhibited by conventional inhalable suspensions comprising a corticosteroid at the same nominal dosage under the same conditions. In still yet another embodiment, the Cₘₐₓ can be at least about 700% (7 fold) greater than the Cₘₐₓ blood plasma values exhibited by conventional inhalable suspensions comprising a corticosteroid at the same nominal dosage under the same conditions. In certain other embodiments, the Cₘₐₓ can be at least about 600% (6 fold) to about 700% (7 fold), about 625% (6.25 fold) to about 700% (7 fold), or about 650% (6.5 fold) to about 700% (7 fold) greater than the Cₘₐₓ blood plasma values exhibited by conventional inhalable suspensions comprising a corticosteroid at the same nominal dosage under the same conditions. In one embodiment, the Cₘₐₓ is at least about 600% (6 fold) greater than the Cₘₐₓ of the inhalable suspension comprising a corticosteroid. In certain other embodiments, the Cₘₐₓ can be at least about 500% (5 fold) to about 600% (6 fold), about 525% (5.25 fold) to about 600% (6 fold), or about 550% (5.5 fold) to about 600% (6 fold) greater than the Cₘₐₓ blood plasma values exhibited by conventional inhalable suspensions comprising a corticosteroid at the same nominal dosage under the same conditions. In another embodiment, the Cₘₐₓ is at least about 500% (5 fold) greater than the Cₘₐₓ of the inhalable suspension comprising a corticosteroid. In certain other embodiments, the Cₘₐₓ can be at least about 400% (4 fold) to about 500% (5 fold), about 425% (4.25 fold) to about 500% (5 fold), or about 450% (4.5 fold) to about 500% (5 fold) greater than the Cₘₐₓ blood plasma values exhibited by conventional inhalable suspensions comprising a corticosteroid at the same nominal dosage under the same conditions. In yet another embodiment, the Cₘₐₓ is at least about 400% (4 fold) greater than the Cₘₐₓ of the inhalable suspension comprising a corticosteroid. In certain other embodiments, the Cₘₐₓ can be at least about 300% (3 fold) to about 400% (4 fold), about 325% (3.25 fold) to about 400% (4 fold), or about 350% (3.5 fold) to about 400% (4 fold) greater than the Cₘₐₓ blood plasma values exhibited by conventional inhalable suspensions comprising a corticosteroid at the same nominal dosage under the same conditions. In still another embodiment, the Cₘₐₓ is at least about 300% (3 fold) greater than the Cₘₐₓ of the inhalable suspension comprising a corticosteroid. In certain other embodiments, the Cₘₐₓ can be at least about 200% (2 fold) to about 300% (3 fold), about 225% (2.25 fold) to about 300% (3 fold), or about 250% (2.5 fold) to about 300% (3 fold) greater than the Cₘₐₓ blood plasma values exhibited by conventional inhalable suspensions comprising a corticosteroid at the same nominal dosage under the same conditions. In yet still another embodiment, the Cₘₐₓ is at least about 200% (2 fold) greater than the Cₘₐₓ of the inhalable suspension comprising a corticosteroid. In certain other embodiments, the Cₘₐₓ can be at about 150% (1.5 fold) to about 200% (2 fold) greater than the Cₘₐₓ blood plasma values exhibited by conventional inhalable suspensions comprising a corticosteroid at the same nominal dosage under the same conditions. In another embodiment, the Cₘₐₓ is at least about 150% (1.5 fold) greater than the Cₘₐₓ of the inhalable suspension comprising a corticosteroid.

In other embodiments, the Cₘₐₓ can be substantially equivalent to the Cₘₐₓ blood plasma values exhibited by conventional inhalable suspensions comprising a corticosteroid wherein the aqueous inhalation mixture comprising a corticosteroid is administered at a lower nominal dosage under the same conditions. In one embodiment, the nominal dosage can be about 1:1.5 (i.e., 1.5 fold enhanced pharmacokinetic profile) to about 1:10 (i.e., 10 fold enhanced pharmacokinetic profile) the nominal dosage of the conventional inhalable suspensions comprising a corticosteroid. In another embodiment, the nominal dosage of the aqueous inhalation mixture comprising a corticosteroid can be about 1:1.5 to about 1:9 of the nominal dosage of the conventional inhalable suspensions comprising a corticosteroid. In yet another embodiment, the nominal dosage of the aqueous inhalation mixture comprising a corticosteroid can be about 1:1.5 to about 1:8 the nominal dosage of the conventional inhalable suspensions comprising a corticosteroid. In still another embodiment, the nominal dosage of the aqueous inhalation mixture comprising a corticosteroid can be about 1:1.5 to about 1:7 the nominal dosage of the conventional inhalable suspensions comprising a corticosteroid. In an additional embodiment, the nominal dosage of the aqueous inhalation mixture comprising a corticosteroid can be about 1:1.5 to about 1:6 the nominal dosage of the conventional inhalable suspensions comprising a corticosteroid. In another embodiment, the nominal dosage of the aqueous inhalation mixture comprising a corticosteroid can be about 1:2 to about 1:5 the nominal dosage of the conventional inhalable suspensions comprising a corticosteroid. In still another embodiment, the nominal dosage of the aqueous inhalation mixture comprising a corticosteroid can be about 1:2 to about 1:4 the nominal dosage of the conventional inhalable suspensions comprising a corticosteroid. In still yet other embodiments, the nominal dosage of the aqueous inhalation mixture comprising a corticosteroid can be about 1:2 to about 1:3 the nominal dosage of the conventional inhalable suspensions comprising a corticosteroid. In certain embodiments, the nominal dosage of the aqueous inhalation mixture comprising a corticosteroid can be about 1:2 the nominal dosage of the conventional inhalable suspensions comprising a corticosteroid. In certain other embodiments, the nominal dosage of the aqueous inhalation mixture comprising a corticosteroid can be about 1:3 the nominal dosage of the conventional inhalable suspensions comprising a corticosteroid. In other certain embodiments, the nominal dosage of the aqueous inhalation mixture comprising a corticosteroid can be about 1:4 the nominal dosage of the conventional inhalable suspensions comprising a corticosteroid. In still other embodiments, the nominal dosage of the aqueous inhalation mixture comprising a corticosteroid can be about 1:5 the nominal dosage of the conventional inhalable suspensions comprising a corticosteroid. In yet still other embodiments, the nominal dosage of the aqueous inhalation mixture comprising a corticosteroid can be about 1:6 the nominal dosage of the conventional inhalable suspensions comprising a corticosteroid. In still yet other embodiments, the nominal dosage of the aqueous inhalation mixture comprising a corticosteroid can be about 1:7 the nominal dosage of the conventional inhalable suspensions comprising a corticosteroid. In other embodiments, the nominal dosage of the aqueous inhalation mixture comprising a corticosteroid can be about 1:8 the nominal dosage of the conventional inhalable suspensions comprising a corticosteroid. In still other embodiments, the nominal dosage of the aqueous inhalation mixture comprising a corticosteroid can be about 1:9 the nominal dosage of the conventional inhalable suspensions comprising a corticosteroid. In yet still other embodiments, the nominal dosage of the aqueous inhalation mixture comprising a corticosteroid can be about 1:10 the nominal dosage of the conventional inhalable suspensions comprising a corticosteroid.

C. AUC₍ₗₐₛₜ₎ Blood Plasma Values

The methods and systems for the treatment or prophylaxis of a bronchoconstrictive disorder in a patient in need thereof described herein can deliver of an inhalation mixture comprising a corticosteroid to the subject in a manner wherein the active is delivered having an increased AUC₍ₗₐₛₜ₎ blood plasma value of the corticosteroid as compared to conventional inhalable corticosteroid suspensions administered under the same conditions. For example, conventional budesonide suspensions administered in a single dose using a Pari LC Plus® jet nebulizer in a 2.0 ml volume with an administration time of about 5 minutes display pharmacokinetic profiles such that the AUC₍ₗₐₛₜ₎ blood plasma values ranges from about 739±220 (pg/h/ml) to about 1989±379 (pg/h/ml) with nominal dosages of 500 μg to 1000 μg, respectfully. Using the systems and methods described herein, budesonide+SBE7-β-CD inhalation solutions having nominal dosages of 60 μg, 120 μg, and 240 μg delivered in a single dose using a Pari eFlow Inhaler® in a 0.5 ml volume with a delivery time of about 1.5 minutes had AUC₍ₗₐₛₜ₎ blood plasma values of about 179±75 (pg/h/ml), about 569±213 (pg/h/ml), and 1183±328 (pg/h/ml), respectively. FIG. 4 provides a graphic representation of the data used to generate the aforementioned AUC₍ₗₐₛₜ₎ blood plasma values.

In a second example, conventional budesonide suspensions (Pulmicort Respules®) administered twice daily for seven days using a Pari LC Plus® jet nebulizer in a 2.0 ml volume with an administration time of about 4 minutes displayed pharmacokinetic profiles having mean AUC₍ₗₐₛₜ₎ blood plasma values of 361.1±212 pg/ml and about 811.1±328 pg/ml with nominal dosages of 250 μg and 500 μg, respectively. The same 250 μg and 500 μg Pulmicort Respules® inhalation suspensions had geometric mean values for AUC₍ₗₐₛₜ₎ blood plasma of 302.6 pg/ml and 735.1 pg/ml, respectively. Using the systems and methods described herein, a 60 μg CBIS inhalation solution delivered twice daily for seven days using a Pari eFlow Inhaler® in a 0.5 ml volume with a delivery time of about 1.5 minutes had a minimum AUC₍ₗₐₛₜ₎ blood plasma value of about 106.4 pg/ml, a maximum AUC₍ₗₐₛₜ₎ blood plasma value of about 463.1 pg/ml, and geometric mean AUC₍ₗₐₛₜ₎ values of about 293.7 pg/ml. Likewise, a 120 μg CBIS inhalation solution delivered twice daily for seven days using a Pari eFlow Inhaler® in a 0.5 ml volume with a delivery time of about 1.5 minutes had a minimum AUC₍ₗₐₛₜ₎ blood plasma value of about 168.2 pg/ml, a maximum AUC₍ₗₐₛₜ₎ blood plasma value of about 1496.7 pg/ml, and geometric mean AUC₍ₗₐₛₜ₎ values of about 621.4 pg/ml. FIG. 5 provides a graphic representation of the data upon which the aforementioned AUC₍ₗₐₛₜ₎ blood plasma values were based.

Thus, the methods and systems for the treatment or prophylaxis of a bronchoconstrictive disorder in a patient in need thereof provide the delivery of an inhalation mixture comprising a corticosteroid having an enhanced pharmacokinetic profile as compared to conventional inhalable corticosteroid suspensions administered under the same conditions. More specifically, the systems and methods described herein provide at least about 1.5 fold to about 10 fold increase in AUC₍ₗₐₛₜ₎ blood plasma values for a corticosteroid (as determined on an individual basis), normalized for dose of corticosteroid per microgram of corticosteroid administered, as compared to conventional inhalable corticosteroid therapies administered under the same conditions. In certain embodiments, the systems and methods described herein provide at least about 1.5 fold to about 10 fold, about 1.5 fold to about 9.5 fold, about 1.5 fold to about 9 fold, about 1.5 fold to about 8.5 fold, about 1.5 fold to about 8 fold, about 1.5 fold to about 7.75 fold, about 1.5 fold to about 7.5 fold, about 1.5 fold to about 7.25 fold, about 1.5 fold to about 7 fold, about 1.5 fold to about 6.75 fold, about 1.5 fold to about 6.5 fold, about 1.5 fold to about 6 fold, about 1.5 fold to about 5.75 fold, about 1.5 fold to about 5.5 fold, about 1.5 fold to about 5 fold, about 1.5 fold to about 4.75 fold, about 1.5 fold to about 4.5 fold, about 1.5 fold to about 4 fold increase in AUC₍ₗₐₛₜ₎ blood plasma values (as determined on an individual basis) for a corticosteroid normalized for dose of corticosteroid per microgram of corticosteroid administered, as compared to conventional inhalable corticosteroid therapies administered under the same condition.

In other embodiments, the methods and systems for the treatment or prophylaxis of a bronchoconstrictive disorder in a patient in need thereof described herein provide at least a about 4 fold to about a 6 fold increase in AUC₍ₗₐₛₜ₎ blood plasma values for a corticosteroid taken across a studied patient population, normalized for dose of corticosteroid per microgram of corticosteroid administered, as compared to conventional inhalable corticosteroid therapies administered under the same conditions, for example as determined using the geometric mean across a studied patient population. In certain embodiments, the systems and methods described herein provide at least about 4 fold to about 6 fold, about 4 fold to about 5.75 fold, about 4 fold to about 5.5 fold, about 4 fold to about 5.25 fold, about 4 fold to about 5 fold, about 4.5 fold to about 6 fold, about 4.75 fold to about 6 fold, about 5 fold to about 6 fold, about 5.5 fold to about 6 fold increase in AUC₍ₗₐₛₜ₎ blood plasma values for a corticosteroid taken across a studied patient population, normalized for dose of corticosteroid per microgram of corticosteroid administered, as compared to conventional inhalable corticosteroid therapies administered under the same conditions.

In some embodiments, the methods and systems for the treatment or prophylaxis of a bronchoconstrictive disorder in a patient in need thereof can provide a AUC₍ₗₐₛₜ₎ that is significantly greater than the AUC₍ₗₐₛₜ₎ blood plasma values exhibited by conventional inhalable suspensions comprising a corticosteroid administered at the same nominal dosage under the same conditions. In certain embodiments, the AUC₍ₗₐₛₜ₎ can be from about 1.5 fold (150%) to about 10 fold (1000%) the AUC₍ₗₐₛₜ₎ blood plasma values exhibited by conventional inhalable suspensions comprising a corticosteroid at the same nominal dosage under the same conditions. In one embodiment, the AUC₍ₗₐₛₜ₎ can be at least about 1000% (10 fold) greater than the AUC₍ₗₐₛₜ₎ blood plasma values exhibited by conventional inhalable suspensions comprising a corticosteroid at the same nominal dosage under the same conditions. In certain other embodiments, the AUC₍ₗₐₛₜ₎ can be at least about 900% (9 fold) to about 1000% (10 fold), about 925% (9.25 fold) to about 1000% (10 fold), or about 950% (9.5 fold) to about 1000% (10 fold) greater than the AUC₍ₗₐₛₜ₎ blood plasma values exhibited by conventional inhalable suspensions comprising a corticosteroid at the same nominal dosage under the same conditions. In another embodiment, the AUC₍ₗₐₛₜ₎ can be at least about 900% (9 fold) greater than the AUC₍ₗₐₛₜ₎ blood plasma values exhibited by conventional inhalable suspensions comprising a corticosteroid at the same nominal dosage under the same conditions. In certain other embodiments, the AUC₍ₗₐₛₜ₎ can be at least about 800% (8 fold) to about 900% (9 fold), about 825% (8.25 fold) to about 900% (9 fold), or about 850% (8.5 fold) to about 900% (9 fold) greater than the AUC₍ₗₐₛₜ₎ blood plasma values exhibited by conventional inhalable suspensions comprising a corticosteroid at the same nominal dosage under the same conditions. In still another embodiment, the AUC₍ₗₐₛₜ₎ can be at least about 800% (8 fold) greater than the AUC₍ₗₐₛₜ₎ blood plasma values exhibited by conventional inhalable suspensions comprising a corticosteroid at the same nominal dosage under the same conditions. In certain other embodiments, the AUC₍ₗₐₛₜ₎ can be at least about 700% (7 fold) to about 800% (8 fold), about 725% (7.25 fold) to about 800% (8 fold), or about 750% (7.5 fold) to about 800% (8 fold) greater than the AUC₍ₗₐₛₜ₎ blood plasma values exhibited by conventional inhalable suspensions comprising a corticosteroid at the same nominal dosage under the same conditions. In still yet another embodiment, the AUC₍ₗₐₛₜ₎ can be at least about 700% (7 fold) greater than the AUC₍ₗₐₛₜ₎ blood plasma values exhibited by conventional inhalable suspensions comprising a corticosteroid at the same nominal dosage under the same conditions. In certain other embodiments, the AUC₍ₗₐₛₜ₎ can be at least about 600% (6 fold) to about 700% (7 fold), about 625% (6.25 fold) to about 700% (7 fold), or about 650% (6.5 fold) to about 700% (7 fold) greater than the AUC₍ₗₐₛₜ₎ blood plasma values exhibited by conventional inhalable suspensions comprising a corticosteroid at the same nominal dosage under the same conditions. In one embodiment, the AUC₍ₗₐₛₜ₎ is at least about 600% (6 fold) greater than the AUC₍ₗₐₛₜ₎ of the inhalable suspension comprising a corticosteroid. In certain other embodiments, the AUC₍ₗₐₛₜ₎ can be at least about 500% (5 fold) to about 600% (6 fold), about 525% (5.25 fold) to about 600% (6 fold), or about 550% (5.5 fold) to about 600% (6 fold) greater than the AUC₍ₗₐₛₜ₎ blood plasma values exhibited by conventional inhalable suspensions comprising a corticosteroid at the same nominal dosage under the same conditions. In another embodiment, the AUC₍ₗₐₛₜ₎ is at least about 500% (5 fold) greater than the AUC₍ₗₐₛₜ₎ of the inhalable suspension comprising a corticosteroid. In certain other embodiments, the AUC₍ₗₐₛₜ₎ can be at least about 400% (4 fold) to about 500% (5 fold), about 425% (4.25 fold) to about 500% (5 fold), or about 450% (4.5 fold) to about 500% (5 fold) greater than the AUC₍ₗₐₛₜ₎ blood plasma values exhibited by conventional inhalable suspensions comprising a corticosteroid at the same nominal dosage under the same conditions. In yet another embodiment, the AUC₍ₗₐₛₜ₎ is at least about 400% (4 fold) greater than the AUC₍ₗₐₛₜ₎ of the inhalable suspension comprising a corticosteroid. In certain other embodiments, the AUC₍ₗₐₛₜ₎ can be at least about 300% (3 fold) to about 400% (4 fold), about 325% (3.25 fold) to about 400% (4 fold), or about 350% (3.5 fold) to about 400% (4 fold) greater than the AUC₍ₗₐₛₜ₎ blood plasma values exhibited by conventional inhalable suspensions comprising a corticosteroid at the same nominal dosage under the same conditions. In still another embodiment, the AUC₍ₗₐₛₜ₎ is at least about 300% (3 fold) greater than the AUC₍ₗₐₛₜ₎ of the inhalable suspension comprising a corticosteroid. In certain other embodiments, the AUC₍ₗₐₛₜ₎ can be at least about 200% (2 fold) to about 300% (3 fold), about 225% (2.25 fold) to about 300% (3 fold), or about 250% (2.5 fold) to about 300% (3 fold) greater than the AUC₍ₗₐₛₜ₎ blood plasma values exhibited by conventional inhalable suspensions comprising a corticosteroid at the same nominal dosage under the same conditions. In yet still another embodiment, the AUC₍ₗₐₛₜ₎ is at least about 200% (2 fold) greater than the AUC₍ₗₐₛₜ₎ of the inhalable suspension comprising a corticosteroid. In certain other embodiments, the AUC₍ₗₐₛₜ₎ can be at about 150% (1.5 fold) to about 200% (2 fold) greater than the AUC₍ₗₐₛₜ₎ blood plasma values exhibited by conventional inhalable suspensions comprising a corticosteroid at the same nominal dosage under the same conditions. In another embodiment, the AUC₍ₗₐₛₜ₎ is at least about 150% (1.5 fold) greater than the AUC₍ₗₐₛₜ₎ of the inhalable suspension comprising a corticosteroid.

In some embodiments, the AUC₍ₗₐₛₜ₎ can be substantially equal to the AUC₍ₗₐₛₜ₎ blood plasma values exhibited by conventional inhalable suspensions comprising a corticosteroid wherein the aqueous inhalation mixture is administered at a lower nominal dosage under the same conditions. In one embodiment, the nominal dosage can be about 1:1.5 (i.e., 1.5 fold enhanced pharmacokinetic profile) to about 1:10 (i.e., 10 fold enhanced pharmacokinetic profile) the nominal dosage of the conventional inhalable suspensions comprising a corticosteroid. In another embodiment, the nominal dosage of the aqueous inhalation mixture comprising a corticosteroid can be about 1:1.5 to about 1:9 of the nominal dosage of the conventional inhalable suspensions comprising a corticosteroid. In yet another embodiment, the nominal dosage of the aqueous inhalation mixture comprising a corticosteroid can be about 1:1.5 to about 1:8 the nominal dosage of the conventional inhalable suspensions comprising a corticosteroid. In still another embodiment, the nominal dosage of the aqueous inhalation mixture comprising a corticosteroid can be about 1:1.5 to about 1:7 the nominal dosage of the conventional inhalable suspensions comprising a corticosteroid. In an additional embodiment, the nominal dosage of the aqueous inhalation mixture comprising a corticosteroid can be about 1:1.5 to about 1:6 the nominal dosage of the conventional inhalable suspensions comprising a corticosteroid. In another embodiment, the nominal dosage of the aqueous inhalation mixture comprising a corticosteroid can be about 1:2 to about 1:5 the nominal dosage of the conventional inhalable suspensions comprising a corticosteroid. In still another embodiment, the nominal dosage of the aqueous inhalation mixture comprising a corticosteroid can be about 1:2 to about 1:4 the nominal dosage of the conventional inhalable suspensions comprising a corticosteroid. In still yet other embodiments, the nominal dosage of the aqueous inhalation mixture comprising a corticosteroid can be about 1:2 to about 1:3 the nominal dosage of the conventional inhalable suspensions comprising a corticosteroid. In certain embodiments, the nominal dosage of the aqueous inhalation mixture comprising a corticosteroid can be about 1:2 the nominal dosage of the conventional inhalable suspensions comprising a corticosteroid. In certain other embodiments, the nominal dosage of the aqueous inhalation mixture comprising a corticosteroid can be about 1:3 the nominal dosage of the conventional inhalable suspensions comprising a corticosteroid. In other certain embodiments, the nominal dosage of the aqueous inhalation mixture comprising a corticosteroid can be about 1:4 the nominal dosage of the conventional inhalable suspensions comprising a corticosteroid. In still other embodiments, the nominal dosage of the aqueous inhalation mixture comprising a corticosteroid can be about 1:5 the nominal dosage of the conventional inhalable suspensions comprising a corticosteroid. In yet still other embodiments, the nominal dosage of the aqueous inhalation mixture comprising a corticosteroid can be about 1:6 the nominal dosage of the conventional inhalable suspensions comprising a corticosteroid. In still yet other embodiments, the nominal dosage of the aqueous inhalation mixture comprising a corticosteroid can be about 1:7 the nominal dosage of the conventional inhalable suspensions comprising a corticosteroid. In other embodiments, the nominal dosage of the aqueous inhalation mixture comprising a corticosteroid can be about 1:8 the nominal dosage of the conventional inhalable suspensions comprising a corticosteroid. In still other embodiments, the nominal dosage of the aqueous inhalation mixture comprising a corticosteroid can be about 1:9 the nominal dosage of the conventional inhalable suspensions comprising a corticosteroid. In yet still other embodiments, the nominal dosage of the aqueous inhalation mixture comprising a corticosteroid can be about 1:10 the nominal dosage of the conventional inhalable suspensions comprising a corticosteroid.

D. AUC_{(0-∞)}Blood Plasma Values

The methods and systems for the treatment or prophylaxis of a bronchoconstrictive disorder in a patient in need thereof described herein can deliver a corticosteroid to the subject in a manner wherein the active is delivered having an increased AUC_{(0-∞)} blood plasma value of the corticosteroid as compared to conventional inhalable corticosteroid suspensions administered under the same conditions. For example, conventional budesonide suspensions administered in a single dose using a Pari LC Plus® jet nebulizer in a 2.0 ml volume with an administration time of about 5 minutes display pharmacokinetic profiles such that the AUC_{(0-∞)} blood plasma values ranges from about 867±216 (pg/h/ml) to about 2083±394 (pg/h/ml) with nominal dosages of 500 μg to 1000 μg, respectfully. Using the systems and methods described herein, budesonide+SBE7-β-CD inhalation solutions having nominal dosages of 60 μg, 120 μg, and 240 μg delivered in a single dose using a Pari eFlow Inhaler® in a 0.5 ml volume with a delivery time of about 1.5 minutes had AUC_{(0-∞)} blood plasma values were about 262±125 (pg/h/ml), about 679±201 (pg/h/ml), and 1365±313 (pg/h/ml), respectively. FIG. 4 provides a graphic representation of the data used to generate the aforementioned AUC₍ₗₐₛₜ₎ blood plasma values.

In a second example, conventional budesonide suspensions (Pulmicort Respules®administered twice daily for seven days using a Pari LC Plus® jet nebulizer in a 2.0 ml volume with an administration time of about 4 minutes displayed pharmacokinetic profiles having mean AUC_{(0-∞)} blood plasma values of 472.3±239 pg/ml and about 945.7±363 pg/ml with nominal dosages of 250 μg and 500 μg, respectively. The same 250 μg and 500 μg Pulmicort Respules® inhalation suspensions had geometric mean values for AUC_{(0-∞)} blood plasma of 413.0 pg/ml and 874.6 pg/ml, respectively. Using the systems and methods described herein, a 60 μg CBIS inhalation solution delivered twice daily for seven days using a Pari eFlow Inhaler® in a 0.5 ml volume with a delivery time of about 1.5 minutes had a minimum AUC_{(0-∞)} blood plasma value of about 156.5 pg/ml, a maximum AUC_{(0-∞)} blood plasma value of about 748.5 pg/ml, and geometric mean AUC_{(0-∞)} values of about 396.1 pg/ml. Likewise, a 120 μg CBIS inhalation solution delivered twice daily for seven days using a Pari eFlow Inhaler® in a 0.5 ml volume with a delivery time of about 1.5 minutes had a minimum AUC_{(0-∞)} blood plasma value of about 221.4 pg/ml, a maximum AUC_{(0-∞)} blood plasma value of about 1863.7 pg/ml, and geometric mean AUC_{(0-∞)} values of about 752.2 pg/ml. FIG. 5 provides a graphic representation of the data upon which the aforementioned AUC_{(0-∞)} blood plasma values were based.

Thus, the methods and systems for the treatment or prophylaxis of a bronchoconstrictive disorder in a patient in need thereof described herein provide the delivery of a corticosteroid having an enhanced pharmacokinetic profile as compared to conventional inhalable corticosteroid suspensions administered under the same conditions. More specifically, the systems and methods described herein provide at least about 1.5 fold to about 10 fold increase in AUC_{(0-∞)} blood plasma values for a corticosteroid (as determined on an individual basis), normalized for dose of corticosteroid per microgram of corticosteroid administered, as compared to conventional inhalable corticosteroid therapies administered under the same conditions. In certain embodiments, the systems and methods described herein provide at least about 1.5 fold to about 10 fold, about 1.5 fold to about 9.5 fold, about 1.5 fold to about 9 fold, about 1.5 fold to about 8.5 fold, about 1.5 fold to about 8 fold, about 1.5 fold to about 7.75 fold, about 1.5 fold to about 7.5 fold, about 1.5 fold to about 7.25 fold, about 1.5 fold to about 7 fold, about 1.5 fold to about 6.75 fold, about 1.5 fold to about 6.5 fold, about 1.5 fold to about 6 fold, about 1.5 fold to about 5.75 fold, about 1.5 fold to about 5.5 fold, about 1.5 fold to about 5 fold, about 1.5 fold to about 4.75 fold, about 1.5 fold to about 4.5 fold, about 1.5 fold to about 4 fold increase in AUC_{(0-∞)} blood plasma values (as determined on an individual basis) for a corticosteroid normalized for dose of corticosteroid per microgram of corticosteroid administered, as compared to conventional inhalable corticosteroid therapies administered under the same condition.

In other embodiments, the methods and systems for the treatment or prophylaxis of a bronchoconstrictive disorder in a patient in need thereof described herein provide at least a about 4 fold to about a 6 fold increase in AUC_{(0-∞)} blood plasma values for a corticosteroid taken across a studied patient population, normalized for dose of corticosteroid per microgram of corticosteroid administered, as compared to conventional inhalable corticosteroid therapies administered under the same conditions, for example as determined using the geometric mean across a studied patient population. In certain embodiments, the systems and methods described herein provide at least about 4 fold to about 6 fold, about 4 fold to about 5.75 fold, about 4 fold to about 5.5 fold, about 4 fold to about 5.25 fold, about 4 fold to about 5 fold, about 4.5 fold to about 6 fold, about 4.75 fold to about 6 fold, about 5 fold to about 6 fold, or about 5.5 fold to about 6 fold increase in AUC_{(0-∞)} blood plasma values for a corticosteroid taken across a studied patient population, normalized for dose of corticosteroid per microgram of corticosteroid administered, as compared to conventional inhalable corticosteroid therapies administered under the same conditions.

In some embodiments, the methods and systems for the treatment or prophylaxis of a bronchoconstrictive disorder in a patient in need thereof can provide a AUC_{(0-∞)} that is significantly greater than the AUC_{(0-∞)} blood plasma values exhibited by conventional inhalable suspensions comprising a corticosteroid administered at the same nominal dosage under the same conditions. In certain embodiments, the AUC_{(0-∞)} can be from about 1.5 fold (150%) to about 10 fold (1000%) the AUC_{(0-∞)} blood plasma values exhibited by conventional inhalable suspensions comprising a corticosteroid at the same nominal dosage under the same conditions. In one embodiment, the AUC_{(0-∞)} can be at least about 1000% (10 fold) greater than the AUC_{(0-∞)} blood plasma values exhibited by conventional inhalable suspensions comprising a corticosteroid at the same nominal dosage under the same conditions. In certain other embodiments, the AUC_{(0-∞)} can be at least about 900% (9 fold) to about 1000% (10 fold), about 925% (9.25 fold) to about 1000% (10 fold), or about 950% (9.5 fold) to about 1000% (10 fold) greater than the AUC_{(0-∞)} blood plasma values exhibited by conventional inhalable suspensions comprising a corticosteroid at the same nominal dosage under the same conditions. In another embodiment, the AUC_{(0-∞)} can be at least about 900% (9 fold) greater than the AUC_{(0-∞)} blood plasma values exhibited by conventional inhalable suspensions comprising a corticosteroid at the same nominal dosage under the same conditions. In certain other embodiments, the AUC_{(0-∞)} can be at least about 800% (8 fold) to about 900% (9 fold), about 825% (8.25 fold) to about 900% (9 fold), or about 850% (8.5 fold) to about 900% (9 fold) greater than the AUC_{(0-∞)} blood plasma values exhibited by conventional inhalable suspensions comprising a corticosteroid at the same nominal dosage under the same conditions. In still another embodiment, the AUC_{(0-∞)} can be at least about 800% (8 fold) greater than the AUC_{(0-∞)} blood plasma values exhibited by conventional inhalable suspensions comprising a corticosteroid at the same nominal dosage under the same conditions. In certain other embodiments, the AUC_{(0-∞)} can be at least about 700% (7 fold) to about 800% (8 fold), about 725% (7.25 fold) to about 800% (8 fold), or about 750% (7.5 fold) to about 800% (8 fold) greater than the AUC_{(0-∞)} blood plasma values exhibited by conventional inhalable suspensions comprising a corticosteroid at the same nominal dosage under the same conditions. In still yet another embodiment, the AUC_{(0-∞)} can be at least about 700% (7 fold) greater than the AUC_{(0-∞)} blood plasma values exhibited by conventional inhalable suspensions comprising a corticosteroid at the same nominal dosage under the same conditions. In certain other embodiments, the AUC_{(0-∞)} can be at least about 600% (6 fold) to about 700% (7 fold), about 625% (6.25 fold) to about 700% (7 fold), or about 650% (6.5 fold) to about 700% (7 fold) greater than the AUC_{(0-∞)} blood plasma values exhibited by conventional inhalable suspensions comprising a corticosteroid at the same nominal dosage under the same conditions. In one embodiment, the AUC_{(0-∞)} is at least about 600% (6 fold) greater than the AUC_{(0-∞)} of the inhalable suspension comprising a corticosteroid. In certain other embodiments, the AUC_{(0-∞)} can be at least about 500% (5 fold) to about 600% (6 fold), about 525% (5.25 fold) to about 600% (6 fold), or about 550% (5.5 fold) to about 600% (6 fold) greater than the AUC_{(0-∞)} blood plasma values exhibited by conventional inhalable suspensions comprising a corticosteroid at the same nominal dosage under the same conditions. In another embodiment, the AUC_{(0-∞)} is at least about 500% (5 fold) greater than the AUC_{(0-∞)} of the inhalable suspension comprising a corticosteroid. In certain other embodiments, the AUC_{(0-∞)} can be at least about 400% (4 fold) to about 500% (5 fold), about 425% (4.25 fold) to about 500% (5 fold), or about 450% (4.5 fold) to about 500% (5 fold) greater than the AUC_{(0-∞)} blood plasma values exhibited by conventional inhalable suspensions comprising a corticosteroid at the same nominal dosage under the same conditions. In yet another embodiment, the AUC_{(0-∞)} is at least about 400% (4 fold) greater than the AUC_{(0-∞)} of the inhalable suspension comprising a corticosteroid. In certain other embodiments, the AUC_{(0-∞)} can be at least about 300% (3 fold) to about 400% (4 fold), about 325% (3.25 fold) to about 400% (4 fold), or about 350% (3.5 fold) to about 400% (4 fold) greater than the AUC_{(0-∞)} blood plasma values exhibited by conventional inhalable suspensions comprising a corticosteroid at the same nominal dosage under the same conditions. In still another embodiment, the AUC_{(0-∞)} is at least about 300% (3 fold) greater than the AUC_{(0-∞)} of the inhalable suspension comprising a corticosteroid. In certain other embodiments, the AUC_{(0-∞)} can be at least about 200% (2 fold) to about 300% (3 fold), about 225% (2.25 fold) to about 300% (3 fold), or about 250% (2.5 fold) to about 300% (3 fold) greater than the AUC_{(0-∞)} blood plasma values exhibited by conventional inhalable suspensions comprising a corticosteroid at the same nominal dosage under the same conditions. In yet still another embodiment, the AUC_{(0-∞)} is at least about 200% (2 fold) greater than the AUC_{(0-∞)} of the inhalable suspension comprising a corticosteroid. In certain other embodiments, the AUC_{(0-∞)} can be at about 150% (1.5 fold) to about 200% (2 fold) greater than the AUC_{(0-∞)} blood plasma values exhibited by conventional inhalable suspensions comprising a corticosteroid at the same nominal dosage under the same conditions. In another embodiment, the AUC_{(0-∞)} is at least about 150% (1.5 fold) greater than the AUC_{(0-∞)} of the inhalable suspension comprising a corticosteroid.

In some embodiments, the AUC_{(0-∞)} can be substantially equal to the AUC_{(0-∞)} blood plasma values exhibited by conventional inhalable suspensions comprising a corticosteroid wherein the aqueous inhalation mixture is administered at a lower nominal dosage under the same conditions. In one embodiment, the nominal dosage can be about 1:1.5 (i.e., 1.5 fold enhanced pharmacokinetic profile) to about 1:10 (i.e., 10 fold enhanced pharmacokinetic profile) the nominal dosage of the conventional inhalable suspensions comprising a corticosteroid. In another embodiment, the nominal dosage of the aqueous inhalation mixture comprising a corticosteroid can be about 1:1.5 to about 1:9 of the nominal dosage of the conventional inhalable suspensions comprising a corticosteroid. In yet another embodiment, the nominal dosage of the aqueous inhalation mixture comprising a corticosteroid can be about 1:1.5 to about 1:8 the nominal dosage of the conventional inhalable suspensions comprising a corticosteroid. In still another embodiment, the nominal dosage of the aqueous inhalation mixture comprising a corticosteroid can be about 1:1.5 to about 1:7 the nominal dosage of the conventional inhalable suspensions comprising a corticosteroid. In an additional embodiment, the nominal dosage of the aqueous inhalation mixture comprising a corticosteroid can be about 1:1.5 to about 1:6 the nominal dosage of the conventional inhalable suspensions comprising a corticosteroid. In another embodiment, the nominal dosage of the aqueous inhalation mixture comprising a corticosteroid can be about 1:2 to about 1:5 the nominal dosage of the conventional inhalable suspensions comprising a corticosteroid. In still another embodiment, the nominal dosage of the aqueous inhalation mixture comprising a corticosteroid can be about 1:2 to about 1:4 the nominal dosage of the conventional inhalable suspensions comprising a corticosteroid. In still yet other embodiments, the nominal dosage of the aqueous inhalation mixture comprising a corticosteroid can be about 1:2 to about 1:3 the nominal dosage of the conventional inhalable suspensions comprising a corticosteroid. In certain embodiments, the nominal dosage of the aqueous inhalation mixture comprising a corticosteroid can be about 1:2 the nominal dosage of the conventional inhalable suspensions comprising a corticosteroid. In certain other embodiments, the nominal dosage of the aqueous inhalation mixture comprising a corticosteroid can be about 1:3 the nominal dosage of the conventional inhalable suspensions comprising a corticosteroid. In other certain embodiments, the nominal dosage of the aqueous inhalation mixture comprising a corticosteroid can be about 1:4 the nominal dosage of the conventional inhalable suspensions comprising a corticosteroid. In still other embodiments, the nominal dosage of the aqueous inhalation mixture comprising a corticosteroid can be about 1:5 the nominal dosage of the conventional inhalable suspensions comprising a corticosteroid. In yet still other embodiments, the nominal dosage of the aqueous inhalation mixture comprising a corticosteroid can be about 1:6 the nominal dosage of the conventional inhalable suspensions comprising a corticosteroid. In still yet other embodiments, the nominal dosage of the aqueous inhalation mixture comprising a corticosteroid can be about 1:7 the nominal dosage of the conventional inhalable suspensions comprising a corticosteroid. In other embodiments, the nominal dosage of the aqueous inhalation mixture comprising a corticosteroid can be about 1:8 the nominal dosage of the conventional inhalable suspensions comprising a corticosteroid. In still other embodiments, the nominal dosage of the aqueous inhalation mixture comprising a corticosteroid can be about 1:9 the nominal dosage of the conventional inhalable suspensions comprising a corticosteroid. In yet still other embodiments, the nominal dosage of the aqueous inhalation mixture comprising a corticosteroid can be about 1:10 the nominal dosage of the conventional inhalable suspensions comprising a corticosteroid.

E. Decreased Tₘₐₓ Blood Plasma Values

The methods and systems for the treatment or prophylaxis of a bronchoconstrictive disorder in a patient in need thereof described herein can deliver a corticosteroid to the subject in a manner wherein the active is delivered having a decreased Tₘₐₓ blood plasma value of the corticosteroid as compared to conventional inhalable corticosteroid suspensions administered at the same dose under the same conditions. In one example, conventional budesonide suspensions administered in a single dose using a Pari LC Plus® jet nebulizer in a 2.0 ml volume with an administration time of about 5 minutes display pharmacokinetic profiles such that the Tₘₐₓ blood plasma values ranges from about 0.24±0.25 (h) to about 0.23±0.24 (h) with nominal dosages of 500 μg to 1000 μg, respectfully. Using the systems and methods described herein, budesonide+SBE7-β-CD inhalation solutions having nominal dosages of 60 μg, 120 μg, and 240 μg delivered in a single dose using a Pari eFlow Inhaler® in a 0.5 ml volume with a delivery time of about 1.5 minutes had Tₘₐₓ blood plasma values of about 0.11±0.09 (h), 0.11±0.09 (h), and 0.21±0.24 (h), respectively.

In a second example, conventional budesonide suspensions (Pulmicort Respules®) administered twice daily for seven days using a Pari LC Plus® jet nebulizer in a 2.0 ml volume with an administration time of about 4 minutes displayed pharmacokinetic profiles having mean Tₘₐₓ blood plasma values of 0.22±0.22 (h) with nominal dosage of 250 μg budesonide, with a minimum Tₘₐₓ blood plasma value of about 0.08 (h) and a maximum Tₘₐₓ blood plasma value of about 0.75 (h), and mean Tₘₐₓ blood plasma values of about 0.19±0.19 (h) with nominal dosage of 500 μg budesonide, with a minimum Tₘₐₓ blood plasma value of about 0.08 (h) and a maximum Tₘₐₓ blood plasma value of about 0.75 (h). Using the systems and methods described herein, a 60 μg CBIS inhalation solution delivered twice daily for seven days using a Pari eFlow Inhaler® in a 0.5 ml volume with a delivery time of about 1.5 minutes had a minimum Tₘₐₓ blood plasma value of about 0.08 (h), a maximum Tₘₐₓ blood plasma value of about 0.25 (h), and mean Tₘₐₓ values of about 0.11 (h). Likewise, a 120 μg CBIS inhalation solution delivered twice daily for seven days using a Pari eFlow Inhaler® in a 0.5 ml volume with a delivery time of about 1.5 minutes had a minimum Tₘₐₓ blood plasma value of about 0.08 (h), a maximum Tₘₐₓ blood plasma value of about 0.50 (h), and mean Tₘₐₓ values of about 0.14 (h). FIG. 5 provides a graphic representation of the data upon which the aforementioned Tₘₐₓ blood plasma values were based.

Thus, the methods and systems for the treatment or prophylaxis of a bronchoconstrictive disorder in a patient in need thereof can provide a Tₘₐₓ that is significantly less than the Tₘₐₓ blood plasma values exhibited by conventional inhalable suspensions comprising a corticosteroid administered at the same nominal dosage under the same conditions. In some embodiments, the Tₘₐₓ can be at least about 1.5 fold to about 10 fold less than the Tₘₐₓ blood plasma values exhibited by conventional inhalable suspensions comprising a corticosteroid administered at the same nominal dosage under the same conditions. In certain embodiments, the Tₘₐₓ can be at least about 8 fold less than the Tₘₐₓ blood plasma values exhibited by conventional inhalable suspensions comprising a corticosteroid administered at the same nominal dosage under the same conditions. In certain other embodiments, the Tₘₐₓ can be at least about 6 fold less than the Tₘₐₓ blood plasma values exhibited by conventional inhalable suspensions comprising a corticosteroid administered at the same nominal dosage under the same conditions. In one such embodiment, the Tₘₐₓ can be at least about 4 fold less than the Tₘₐₓ blood plasma values exhibited by conventional inhalable suspensions comprising a corticosteroid administered at the same nominal dosage under the same conditions. In another embodiment, the Tₘₐₓ can be at least about 3 fold less than the Tₘₐₓ blood plasma values exhibited by conventional inhalable suspensions comprising a corticosteroid administered at the same nominal dosage under the same conditions. In still another embodiment, the Tₘₐₓ can be at least about 2 fold faster than the Tₘₐₓ blood plasma values exhibited by conventional inhalable suspensions comprising a corticosteroid administered at the same nominal dosage under the same conditions. In yet still another embodiment, the Tₘₐₓ can be at least about 1.5 fold faster than the Tₘₐₓ blood plasma values exhibited by conventional inhalable suspensions comprising a corticosteroid administered at the same nominal dosage under the same conditions.

In some embodiments, the Tₘₐₓ can be less than the Tₘₐₓ blood plasma values exhibited by conventional inhalable suspensions comprising a corticosteroid wherein the aqueous inhalation mixture is administered at a lower nominal dosage under the same conditions. In one embodiment, the nominal dosage can be about 1:1.5 (i.e., 1.5 fold enhanced pharmacokinetic profile) to about 1:10 (i.e., 10 fold enhanced pharmacokinetic profile) the nominal dosage of the conventional inhalable suspensions comprising a corticosteroid. In another embodiment, the nominal dosage of the aqueous inhalation mixture comprising a corticosteroid can be about 1:1.5 to about 1:9 of the nominal dosage of the conventional inhalable suspensions comprising a corticosteroid. In yet another embodiment, the nominal dosage of the aqueous inhalation mixture comprising a corticosteroid can be about 1:1.5 to about 1:8 the nominal dosage of the conventional inhalable suspensions comprising a corticosteroid. In still another embodiment, the nominal dosage of the aqueous inhalation mixture comprising a corticosteroid can be about 1:1.5 to about 1:7 the nominal dosage of the conventional inhalable suspensions comprising a corticosteroid. In an additional embodiment, the nominal dosage of the aqueous inhalation mixture comprising a corticosteroid can be about 1:1.5 to about 1:6 the nominal dosage of the conventional inhalable suspensions comprising a corticosteroid. In another embodiment, the nominal dosage of the aqueous inhalation mixture comprising a corticosteroid can be about 1:2 to about 1:5 the nominal dosage of the conventional inhalable suspensions comprising a corticosteroid. In still another embodiment, the nominal dosage of the aqueous inhalation mixture comprising a corticosteroid can be about 1:2 to about 1:4 the nominal dosage of the conventional inhalable suspensions comprising a corticosteroid. In still yet other embodiments, the nominal dosage of the aqueous inhalation mixture comprising a corticosteroid can be about 1:2 to about 1:3 the nominal dosage of the conventional inhalable suspensions comprising a corticosteroid. In certain embodiments, the nominal dosage of the aqueous inhalation mixture comprising a corticosteroid can be about 1:2 the nominal dosage of the conventional inhalable suspensions comprising a corticosteroid. In certain other embodiments, the nominal dosage of the aqueous inhalation mixture comprising a corticosteroid can be about 1:3 the nominal dosage of the conventional inhalable suspensions comprising a corticosteroid. In other certain embodiments, the nominal dosage of the aqueous inhalation mixture comprising a corticosteroid can be about 1:4 the nominal dosage of the conventional inhalable suspensions comprising a corticosteroid. In still other embodiments, the nominal dosage of the aqueous inhalation mixture comprising a corticosteroid can be about 1:5 the nominal dosage of the conventional inhalable suspensions comprising a corticosteroid. In yet still other embodiments, the nominal dosage of the aqueous inhalation mixture comprising a corticosteroid can be about 1:6 the nominal dosage of the conventional inhalable suspensions comprising a corticosteroid. In still yet other embodiments, the nominal dosage of the aqueous inhalation mixture comprising a corticosteroid can be about 1:7 the nominal dosage of the conventional inhalable suspensions comprising a corticosteroid. In other embodiments, the nominal dosage of the aqueous inhalation mixture comprising a corticosteroid can be about 1:8 the nominal dosage of the conventional inhalable suspensions comprising a corticosteroid. In still other embodiments, the nominal dosage of the aqueous inhalation mixture comprising a corticosteroid can be about 1:9 the nominal dosage of the conventional inhalable suspensions comprising a corticosteroid. In yet still other embodiments, the nominal dosage of the aqueous inhalation mixture comprising a corticosteroid can be about 1:10 the nominal dosage of the conventional inhalable suspensions comprising a corticosteroid.

XIII. Dosages for Use in Methods and Systems for Treatment

The methods and systems for the treatment or prophylaxis of a bronchoconstrictive disorder in a patient in need thereof can deliver an aqueous inhalation mixture comprising a corticosteroid, e.g., budesonide, and a solubility enhancer to the subject in a manner wherein the active is delivered in accordance with good medical practice, taking into account the clinical condition of the individual patient, the site and method of administration, scheduling of administration, and other factors known to medical practitioners. In human therapy, the methods described herein can deliver corticosteroid solutions, e.g., a budesonide solution, that maintain a therapeutically effective amount of the corticosteroid, e.g., budesonide, at the site of action which reduces or mitigates symptoms related to bronchoconstrictive disorders. In other embodiments the aqueous inhalation mixture comprises a corticosteroid and a solubility enhancer, wherein the inhalation mixture is substantially free of pharmaceutically active agents other than a corticosteroid.

In other embodiments, the methods and systems for the treatment or prophylaxis of a bronchoconstrictive disorder in a patient in need thereof described herein can deliver an aqueous inhalation mixture comprising a corticosteroid, e.g., budesonide, a solvent, and a solubility enhancer to the subject in a manner wherein the active is delivered in accordance with good medical practice, taking into account the clinical condition of the individual patient, the site and method of administration, scheduling of administration, and other factors known to medical practitioners. In human therapy, the methods described herein can deliver corticosteroid solutions, e.g., a budesonide solution, that maintain a therapeutically effective amount of the corticosteroid, e.g., budesonide, at the site of action which reduces or mitigates symptoms related to bronchoconstrictive disorders. In other embodiments the aqueous inhalation mixture comprises a corticosteroid, a solvent, and a solubility enhancer, wherein the inhalation mixture is substantially free of pharmaceutically active agents other than a corticosteroid.

In various embodiments of the methods and systems described herein above in Section XII, the methods and systems for the treatment or prophylaxis of a bronchoconstrictive disorder in a patient in need thereof described herein can deliver an aqueous inhalation mixture comprising a therapeutically effective amount of a corticosteroid administered to a subject via an inhalation nebulizer at a nominal dosage in the range of about 15 μg/dose to less than about 250 μg/dose, or from about 25 μg/dose to about 240 μg/dose, or from about 200 μg/dose to about 240 μg/dose, or from about 125 μg/dose to about 200 μg/dose, or from about 150 μg/dose to about 200 μg/dose, or from about 100 μg/dose to about 150 μg/dose, or from about 100 μg/dose to about 125 μg/dose, or from about 50 μg/dose to about 125 μg/dose, or from about 60 μg/dose to about 125 μg/dose, or from about 25 μg/dose to about 50 μg/dose. In a preferred embodiment, the corticosteroid is budesonide administered to a subject via an inhalation nebulizer at a nominal dosage in the range of about 25 μg/dose to about 240 μg/dose. In one embodiment, the aqueous inhalation mixture comprises a corticosteroid, such as budesonide, wherein the aqueous inhalation mixture is administered according to the methods and systems described herein with a nominal dosage of the corticosteroid of about 60 μg/dose to less than about 250 μg/dose. In another embodiment, the aqueous inhalation mixture comprises a corticosteroid, such as budesonide, wherein the aqueous inhalation mixture is administered according to the methods and systems described herein with a nominal dosage of the corticosteroid of less than about 250 μg/dose. In still another embodiment, the aqueous inhalation mixture comprises a corticosteroid, such as budesonide, wherein the aqueous inhalation mixture is administered according to the methods and systems described herein with a nominal dosage of the corticosteroid of about 240 μg/dose. In yet another embodiment, the aqueous inhalation mixture comprises a corticosteroid, such as budesonide, wherein the aqueous inhalation mixture is administered according to the methods and systems described herein with a nominal dosage of the corticosteroid of about 125 μg/dose. In still another embodiment, the aqueous inhalation mixture comprises a corticosteroid, such as budesonide, wherein the aqueous inhalation mixture is administered according to the methods and systems described herein with a nominal dosage of the corticosteroid of about 120 μg/dose. In yet still another embodiment, the aqueous inhalation mixture comprises a corticosteroid, such as budesonide, wherein the aqueous inhalation mixture is administered according to the methods and systems described herein with a nominal dosage of the corticosteroid of about 60 μg/dose. In still another embodiment, the aqueous inhalation mixture comprises a corticosteroid, such as budesonide, wherein the aqueous inhalation mixture is administered according to the methods and systems described herein with a nominal dosage of the corticosteroid of about 40 μg/dose. In certain embodiments, the aqueous inhalation mixture comprises a single corticosteroid and is substantially free of pharmaceutically active agents other than the corticosteroid.

In certain embodiments, the methods and systems for the treatment or prophylaxis of a bronchoconstrictive disorder in a patient in need thereof can deliver an aqueous inhalation mixture comprising a therapeutically effective amount of a corticosteroid administered to a subject via an inhalation nebulizer at a nominal dosage in the range of about 25 μg/dose to less than about 100 μg/dose wherein the the corticosteroid is selected group of corticosteroids in the foregoing paragraph not including the betamethasone. In one such embodiment, the aqueous inhalation mixture comprises a corticosteroid, such as budesonide, wherein the aqueous inhalation mixture is administered according to the methods and systems described herein with a nominal dosage of the corticosteroid of less than about 100 μg/dose. In another embodiment, the aqueous inhalation mixture comprises a corticosteroid, such as budesonide, wherein the aqueous inhalation mixture is administered according to the methods and systems described herein with a nominal dosage of the corticosteroid of about 60 μg/dose. In still another embodiment, the aqueous inhalation mixture comprises a corticosteroid, such as budesonide, wherein the aqueous inhalation mixture is administered according to the methods and systems described herein with a nominal dosage of the corticosteroid of about 40 μg/dose. In certain embodiments, the aqueous inhalation mixture comprises a single corticosteroid and is substantially free of pharmaceutically active agents other than the corticosteroid.

In some embodiments of the inhalable compositions or aqueous inhalation mixtures described herein, the inhalable composition or aqueous inhalation mixture comprises a solvent. In certain embodiments, the solvent is selected from the group comprising water, aqueous alcohol, propylene glycol, or aqueous organic solvent. In preferred embodiments, the solvent is water.

In some embodiments of the systems and methods described herein, a corticosteroid-containing aqueous inhalation mixture is employed which further comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 2% to about 10% when the solubility enhancer is a cyclodextrin or cyclodextrin derivative, e.g. SBE7-β-CD (Captisol®). In one embodiment, the solubility enhancer can have a concentration (w/v) of about 2% when the solubility enhancer is a cyclodextrin or cyclodextrin derivative, e.g. SBE7-β-CD (Captisol®). In another embodiment, the solubility enhancer can have a concentration (w/v) of about 5% when the solubility enhancer is a cyclodextrin or cyclodextrin derivative, e.g. SBE7-β-CD (Captisol®). In yet another embodiment, the solubility enhancer can have a concentration (w/v) about 7% when the solubility enhancer is a cyclodextrin or cyclodextrin derivative, e.g. SBE7-β-CD (Captisol®). In still yet another embodiment, the solubility enhancer can have a concentration (w/v) of about 10% when the solubility enhancer is a cyclodextrin or cyclodextrin derivative, e.g. SBE7-β-CD (Captisol®).

In certain embodiments, the aqueous inhalation mixture comprises a solubility enhancer selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In certain embodiments, the solubility enhancer is SBE7-β-CD (Captisol®).

In certain other embodiments, the inhalable compositions of the present invention comprise a solubility enhancer selected from the group consisting of cyclodextrins and derivatives thereof, SAE-CD derivatives, SBE-α-CD, SBE-β-CD, SBE1-β-CD, SBE4-β-CD, SBE7-β-CD (Captisol®), SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin. In certain embodiments, the solubility enhancer is SBE7-β-CD (Captisol®)).

In addition to aqueous inhalation mixtures or inhalable composition comprising a corticosteroid and a solubility enhancer, it is contemplated herein that aqueous inhalation mixtures or compositions formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein.

In another aspect, the aqueous inhalation mixture comprising a corticosteroid is administered according to the methods and systems described herein not more than twice a day (b.i.d). In still another aspect, the aqueous inhalation mixture comprises a corticosteroid, such as budesonide, wherein the aqueous inhalation mixture is administered according to the methods and systems described herein twice a day. In yet another aspect, the aqueous inhalation mixture comprises a corticosteroid, such as budesonide, wherein the aqueous inhalation mixture is administered according to the methods and systems described herein not more than once a day. In still another embodiment, the aqueous inhalation mixture comprises a corticosteroid, such as budesonide, wherein the aqueous inhalation mixture is administered according to the methods and systems described herein not more than once a day in the evening.

In certain embodiments, the methods and systems described herein can further include administering aqueous inhalation mixtures comprising a corticosteroid in combination with one or more active agents. In some embodiments, the corticosteroid, e.g., budesonide, can be administered in combination with one or more other drugs one or more active agents selected from the group consisting of: (a) a B2-adrenoreceptor agonist; (b) a dopamine (D2) receptor agonist; (c) a prophylactic therapeutic, such as a steroid; (d) a topical anesthetic; or (e) an anti-cholinergic agent; either simultaneously with, prior to or subsequent to the inhalable composition provided herein.

Examples of combinations of active agents which can be used in the methods and systems of the present invention are provided below.

B2-Adrenoreceptor agonists for use in combination with the compositions provided herein include, but are not limited to, Albuterol (α1-(((1,1-dimethylethyl)amino)methyl)-4-hydroxy-1,3-benzenedimethanol); Bambuterol (dimethylcarbamic acid 5-(2-((1,1-dimethylethyl)amino)-1-hydroxyethyl)-1,3-phenyleneester); Bitolterol (4-methylbenzoic acid-4-(2-((1,1-dimethylethyl)amino)-1-hydroxyethyl)-1,2-phenyleneester); Broxaterol (3-bromo-alpha-(((1,1-dimethylethyl)amino)methyl)-5-isoxazolemethanol); Isoproterenol (4-(1-hydroxy-2-((1-methylethyl-)amino)ethyl)-1,2-benzene-diol); Trimetoquinol (1,2,3,4-tetrahydro-1-((3,4-,5-trimethoxyphenyl)-methyl)-6,7-isoquinolinediol); Clenbuterol (4-amino-3,5-dichloro-alpha-(((1,1-dimethylethyl)amino)methyl)benzenemethanol); Fenoterol (5-(1-hydroxy-2-((2-(4-hydroxyphenyl)-1-methylethyl)amino)ethyl)-1,3-benzenediol); Formoterol (2-hydroxy-5-((1RS)-1-hydroxy-2-(((1RS)-2-(p-methoxyphenyl)-1-methylethyl)amino)ethyl)formanilide); (R,R)-Formoterol; Desformoterol ((R,R) or (S,S)-3-amino-4-hydroxy-alpha-(((2-(4-methoxyphenyl)-1-methyl-ethyl)amino)methyl)benzenemethanol); Hexoprenaline (4,4′-(1,6-hexane-diyl)-bis(imino(1-hydroxy-2,1-ethanediyl)))bis-1,2-benzenediol); Isoetharine (4-(1-hydroxy-2-((1-methylethyl)amino)butyl)-1,2-benzenediol); Isoprenaline (4-(1-hydroxy-2-((1-methylethyl)amino)ethyl)-1,2-benzenediol); Meta-proterenol (5-(1-hydroxy-2-((1-methylethyl)amino)ethyl)-1,3-benzenediol); Picumeterol (4-amino-3,5-dichloro-α-(((6-(2-(2-pyridinyl)ethoxy)hexyl)-amino)methyl)benzenemethanol); Pirbuterol (α-6-(((1,1-dimethylethyl)-amino)methyl)-3-hydroxy-2,6-pyridinemethanol); Procaterol (((R*,S*)-(+-)-8-hydroxy-5-(1-hydroxy-2-((1-methylethyl)amino)butyl)-2(1H)-quinolin-one); Reproterol ((7-(3-((2-(3,5-dihydroxyphenyl)-2-hydroxyethyl)amino)-propyl)-3,7-dihydro-1,3-dimethyl-1H-purine-2,6-dione); Rimiterol (4-(hydroxy-2-piperidinylmethyl)-1,2-benzenediol); Salbutamol ((+-)-alpha1-(((1,1-dimethylethyl)amino)methyl)-4-hydroxy-1,3-benzenedimethanol); (R)-Salbutamol; Salmeterol ((+-)-4-hydroxy-α-1-(((6-(4-phenylbutoxy)hexyl)-amino)methyl)-1,3-benzenedimethanol); (R)-Salmeterol; Terbutaline (5-(2-((1,1-dimethylethyl)amino)-1-hydroxyethyl)-1,3-benzenediol); Tulobuterol (2-chloro-α-(((1,1-dimethylethyl)amino)methyl)benzenemethanol); and TA-2005 (8-hydroxy-5-((1R)-1-hydroxy-2-(N-((1R)-2-(4-methoxyphenyl)-1-methylethyl)amino)ethyl)carbostyril hydrochloride).

Dopamine (D2) receptor agonists include, but are not limited to, Apomorphine ((r)-5,6,6a,7-tetrahydro-6-methyl-4H-dibenzo[de,gl quinoline-10,11-diol); Bromocriptine ((5′.alpha.)-2-bromo-12′-hydroxy-2′-(1-methylethyl)-5′-(2-methylpropyl)ergotaman-3′,6′,18-trione); Cabergoline ((8-β-N-(3-(dimethylamino)propyl)-N-((ethylamino)carboxy-1)-6-(2-propenyl)ergoline-8-carboxamide); Lisuride (N′-((8-α-)-9,10-didehydro-6-methylergolin-8-yl)-N,N-diethylurea); Pergolide ((8-β-)-8-((methylthio)methyl)-6-propylergoline); Levodopa (3-hydroxy-L-tryrosine); Pramipexole ((s)-4,5,6,7-tetrahydro-N6-propyl-2,6-benzothiazolediamine); Quinpirole hydrochloride (trans-(−)-4aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thia-zolo[4,5-d]azepin-2-amine). Other dopamine D2 receptor agonists for use herein are disclosed in International Patent Application Publication No. WO 99/36095, the relevant disclosure of which is hereby incorporated by reference.

Anti-cholinergic agents for use herein include, but are not limited to, ipratropium bromide, oxitropium bromide, atropine methyl nitrate, atropine sulfate, ipratropium, belladonna extract, scopolamine, scopolamine methobromide, homatropine methobromide, hyoscyamine, isopriopramide, orphenadrine, benzalkonium chloride, tiotropium bromide and glycopyrronium bromide.

Other active ingredients for use herein in combination therapy, include, but are not limited to, IL-5 inhibitors such as those disclosed in U.S. Pat. No. 5,668,110, No. 5,683,983, No. 5,677,280, No. 6,071,910 and No. 5,654,276, each of which is incorporated by reference herein; anti-sense modulators of IL-5 such as those disclosed in U.S. Pat. No. 6,136,603, the relevant disclosure of which is hereby incorporated by reference; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623, which is incorporated by reference herein; tachykinin receptor antagonists such as those disclosed in U.S. Pat. No. 5,691,336, No. 5,877,191, No. 5,929,094, No. 5,750,549 and No. 5,780,467, each of which is incorporated by reference herein; leukotriene receptor antagonists such as montelukast sodium (Singular, R-(E)]-1-[[[1-[3-[2-(7-chloro-2-quinolinyl)-ethenyl]-phenyl]-3-[2-(1-hydroxy-1-methylethyl)-phenyl]-propyl]-thio]-methyl]cyclopropaneacetic acid, monosodium salt), 5-lypoxygenase inhibitors such as zileuton (Zyflo®, Abbott Laboratories, Abbott Park, Ill.), and anti-IgE antibodies such as Xolair (recombinant humanized anti-IgE monoclonal antibody (CGP 51901; IGE 025A; rhuMAb-E25), Genentech, Inc., South San Francisco, Calif.), and topical anesthetics such as lidocaine, N-arylamide, aminoalkylbenzoate, prilocaine, etidocaine (U.S. Pat. No. 5,510,339, No. 5,631,267, and No. 5,837,713, the relevant disclosures of which are hereby incorporated by reference).

XIV. Methods of Use of Aqueous Inhalation Mixtures Comprising a Corticosteroid

The methods and systems described herein above in Section XII herein can deliver an aqueous inhalable mixture comprising a corticosteroid, e.g. budesonide, to a subject in therapeutically effective amount for the treatment of a subject that has had or is anticipating a bronchoconstrictive disorder selected from the group consisting of asthma, pediatric asthma, bronchial asthma, allergic asthma, intrinsic asthma, chronic obstructive pulmonary disease (COPD), chronic bronchitis, emphysema, or a combination of any of the above. In one embodiment, the bronchoconstrictive disorder is pediatric asthma. In another embodiment, the bronchoconstrictive disorder is bronchial asthma. In still another embodiment, the bronchoconstrictive disorder is chronic obstructive pulmonary disease (COPD).

Actual dosage levels of the aqueous inhalation mixtures comprising a corticosteroid described herein may be varied to obtain an amount of active ingredient that is effective to obtain a desired local therapeutic response for a particular composition and method of administration. The selected dosage level therefore depends upon the desired therapeutic effect, on the desired duration of treatment, and other factors.

XV. Pharmacokinetic Analysis

Any standard pharmacokinetic protocol can be used to determine blood plasma concentration profile in humans following administration of an aqueous inhalation solution comprising a corticosteroid, such as a budesonide, and a solubility enhancer by the systems and methods described herein, and thereby establish whether that formulation meets the pharmacokinetic criteria set out herein. For example, but in no way limiting the type of protocols for use herein, a randomized single-dose crossover study can be performed using a group of healthy adult human subjects. The number of subjects should be sufficient to provide adequate control of variation in a statistical analysis, and is typically about 8 or greater, although for certain purposes a smaller group can suffice. For example, subject receives administration at time zero a single dose (e.g., 240 μg) of a test inhalation mixture comprising a corticosteroid, such as a budesonide, and a solubility enhancer. Blood samples are collected from each subject prior to administration (e.g., 15 minutes) and at several intervals after administration. For the present purpose it is typically preferred to take several samples within the first hour and to sample less frequently thereafter. Illustratively, blood samples could be collected at 5, 10, 20, 30, 45, and 60 minutes after administration, and then at 2, 4, 8, and 12 hours after administration. If the same subjects are to be used for study of a second test formulation, a period of at least 10 days should elapse before administration of the second formulation. Plasma is separated from the blood samples by centrifugation and the separated plasma is analyzed for a corticosteroid, such as a budesonide, by a validated high performance liquid chromatography/tandem weight spectrometry (LC/APCI-MS/MS) procedure such as, for example, Ramu et al., Journal of Chromatography B, 751:49-59 (2001). In other embodiments, data from a single subject may be indicative of an enhanced pharmacokinetic profile. In still other embodiments, appropriate in vitro models may be used to demonstrate enhanced pharmacokinetic profiles.

Any aqueous inhalable mixture giving the desired pharmacokinetic profile is suitable for administration according to the present systems and methods. Exemplary types of inhalable mixtures giving such profiles are solutions comprising a corticosteroid, such as a budesonide, and a solubility enhancer.

### EXAMPLES

The following ingredients, processes and procedures for practicing the compositions, systems and methods disclosed herein correspond to that described above. The procedures below describe specific embodiments of methods of delivery of an aqueous inhalation mixture comprising budesonide as described herein and pharmacokinetic profiles thereof. Methods, materials, or excipients which are not specifically described in the following examples are within the scope of the invention and will be apparent to those skilled in the art with reference to the disclosure herein.

### Example 1

Multiple aqueous inhalation mixtures were prepared by discharging the contents of one or more containers of commercially available Pulmicort Respules® (1000 μg budesonide per 2 mL of the suspension), and 82.5 mg (corrected for water content) of Captisol® (CyDex, Inc., Lenexa, Kans., USA) was added per mL of Pulmicort Respules® (dispensed volume was 2.1 mL) and vortexed for 5-10 minutes. In addition to budesonide and water, the Pulmicort Respules® also contain the following ingredients which are believed to be inactive: citric acid, sodium citrate, sodium chloride, disodium EDTA, and polysorbate 80.

### Example 2

As an alternative method of preparation to Example 1, multiple aqueous inhalation mixtures are prepared by weighing approximately 200 mg amounts of CAPTISOL® (CyDex, Inc., Lenexa, Kans., USA) (corrected for water content) into 2-dram amber vials. Into each vial containing the weighed amount of CAPTISOL, the contents of two Pulmicort Respules® containers (0.5 mg/2 mL) are emptied by gently squeezing the deformable plastic container to the last possible drop. The Pulmicort Respules® are previously swirled to re-suspend the budesonide particles. The vials are screw capped, mixed vigorously by vortex and then foil wrapped. The material can be kept refrigerated until use.

### Example 3

Table 1 provides exemplary formulations of aqueous inhalation mixtures comprising budesonide and a solubility enhancer which are used in the methods and systems described herein. As indicated by the following example, the aqueous inhalation mixtures can further comprise excipients, e.g., antioxidants, stabilizing agents, and preservatives. The amount of the various excipients to be used in the aqueous inhalation mixture will be relative to the dosage to be administered and will be readily ascertained by a person having ordinary skill in the art.

TABLE 1

| Starting Material | | | | | |
|---|---|---|---|---|---|
| (mg) | 1% DM-β-CD | 5% DM-β-CD | 7% DM-β-CD | 5% DM-β-CD | 5% DM-β-CD |
| Budesonide | 0.024 | 0.024 | 0.024 | 0.006 | 0.012 |
| Di methyl-β-CD | 1.0 | 5.0 | 7.0 | 5.0 | 5.0 |
| Citric Acid | 0.045 | 0.045 | 0.045 | 0.045 | 0.045 |
| Sodium Chloride | 0.850 | 0.850 | 0.850 | 0.850 | 0.850 |
| Disodium edetate | 0.05 | 0.05 | 0.05 | 0.05 | 0.05 |
| Water | Ad 100.0 | Ad 100.0 | Ad 100.0 | Ad 100.0 | Ad 100.0 |

### Example 4

Table 2 provides exemplary formulations of aqueous inhalation mixtures comprising budesonide and a solubility enhancer which are used in the methods and systems described herein. As indicated by the following example, the aqueous inhalation mixtures can further comprise excipients, e.g., antioxidants, stabilizing agents, and preservatives. The amount of the various excipients to be used in the aqueous inhalation mixture will be relative to the dosage to be administered and will be readily ascertained by a person having ordinary skill in the art.

TABLE 2

| Starting Material | | | | | |
|---|---|---|---|---|---|
| (mg) | 1% HP-β-CD | 5% HP-β-CD | 7% HP-β-CD | 5% HP-β-CD | 5% HP-β-CD |
| Budesonide | 0.024 | 0.024 | 0.024 | 0.006 | 0.012 |
| HP-β-CD | 1.0 | 5.0 | 0.7 | 5.0 | 5.0 |
| Citric Acid | 0.045 | 0.045 | 0.045 | 0.045 | 0.045 |
| Sodium Chloride | 0.850 | 0.850 | 0.850 | 0.850 | 0.850 |
| Disodium edetate | 0.05 | 0.05 | 0.05 | 0.05 | 0.05 |
| Water | Ad 100.0 | Ad 100.0 | Ad 100.0 | Ad 100.0 | Ad 100.0 |

### Example 5

Set forth in Table 3 are doses of aqueous inhalation mixtures comprising budesonide and Pulmicort Respules®, and the nebulizer devices used for the delivery to the lung of said doses. Administrations A-C were prepared by diluting the aqueous inhalation mixtures prepared as described in Example 1 with 0.9% (w/w) saline in the following manner: Administration A was diluted at a 25:75 ratio with 0.9% (w/w) saline; Administration B was diluted at a 50:50 ratio with 0.9% (w/w) saline; and Administration C was not diluted. The budesonide+SBE7-β-CD (Captisol®) inhalation solutions were delivered using a Pari eFlow Inhaler® in a 0.5 ml volume with a delivery time of about 1.5 minutes. The Pari eFlow Inhaler® was fitted with a size 30 mesh membrane and a small size aerosol chamber. The Pulmicort Respules® were administered using a Pari LC Plus® jet nebulizer in a 2.0 ml volume with an administration time of about 5 minutes.

TABLE 3

| Study | | | |
|---|---|---|---|
| No. | Admin. | Dose | Inhaler |
| 1 | A | 60 μg 99mTc-DTPA labeled budesonide + | Pari eFlow |
| | | SBE7-β-CD inhalation solution | inhaler |
| 2 | B | 120 μg 99mTc-DTPA labeled budesonide + | Pari eFlow |
| | | SBE7-β-CD inhalation solution | inhaler |
| 3 | C | 240 μg 99mTc-DTPA labeled budesonide + | Pari eFlow |
| | | SBE7-β-CD inhalation solution | inhaler |
| 4 | D | 500 μg budesonide suspension | Pari LC |
| | | (Pulmicort Respules ®) | Plus jet |
| | | | nebulizer |
| 5 | E | 1000 μg budesonide suspension | Pari LC |
| | | (Pulmicort Respules ®) | Plus jet |
| | | | nebulizer |

### Example 6

Clinical evaluation was conducted by performing gamma scintigraph analysis on subjects before and after administration of the dosage form by nebulization. The purpose of the study is to determine, by gamma scintigraphy, the intra-pulmonary deposition of radiolabeled budesonide following nebulization of a budesonide suspension or a solution with a solubility enhancer.

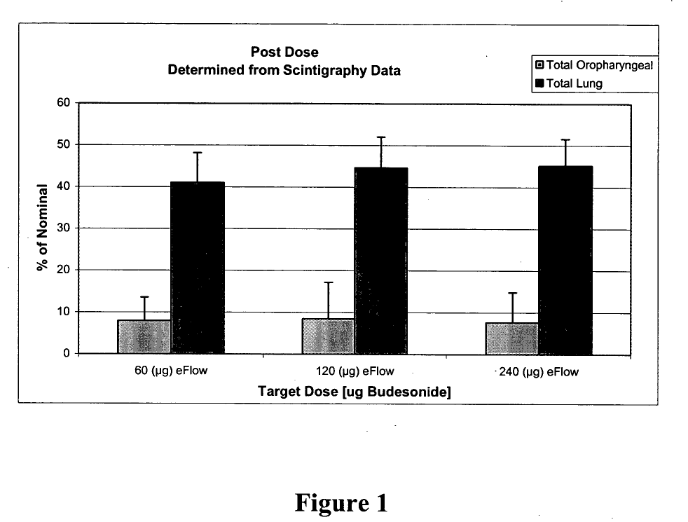

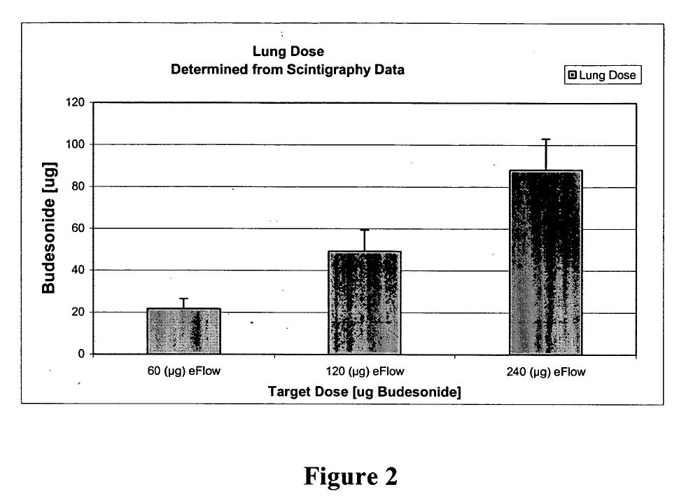

Set forth in Table 4 is a summary of the data related to percent lung deposition for Administrations A to C as delivered by the methods described in Example 5. Percent lung deposition is the mean value for all subjects evaluated and was determined by quantification of scintigraphy data taken for each Administration. FIG. 1 shows percentage of total lung deposition and propharyngeal deposition of an inhalable composition comprising budesonide. FIG. 2 shows total lung deposition of budesonide from scintigraphy data.

TABLE 4

| | Target | | | Percent |
|---|---|---|---|---|
| | Dose | True nominal | Corrected Lung | Lung |
| Admin | (μg) | Dose (μg) | Dose (μg) | Deposition |
| A | 60 | 60.69 ± 1.95 | 21.72 ± 4.81 | 35.7 |
| B | 120 | 121.16 ± 4.29 | 49.26 ± 10.13 | 40.6 |
| C | 240 | 234.85 ± 2.74 | 88.13 ± 14.93 | 37.5 |

### Example 7

Set forth in Table 5 is the summary of the pharmacokinetic profiles for budesonide following a single dose delivery of Administrations A to E as set forth in Example 5. Eight (8) healthy males were used in this clinical study and the values presented below are the mean values for each of the pharmacokinetic parameters measured during the clinical study. FIG. 4 provides a graphic representation of the data used to generate the pharmacokinetic profiles for Administrations A to E.

TABLE 5

| | | Cₘₐₓ | | AUC(ₗₐₛₜ) | AUC_{(0-INF)} | |
|---|---|---|---|---|---|---|
| Administration | | (pg/ml) | Tₘₐₓ (h) | (pg/ml/h) | (pg/ml/h) | T_{1/2λz} (h) |
| Admin A (60 μg BUD- | Mean | 227.6 | 0.11 | 179.17 | 262.93 | 1.24 |
| SBE7-β-CD, Pari eFlow) | SD | 89.67 | 0.09 | 75.47 | 125.14 | 0.59 |
| Admin B (120 μg BUD- | Mean | 578.2 | 0.11 | 569.68 | 679.85 | 2.08 |
| SBE7-β-CD, Pari eFlow) | SD | 238.69 | 0.09 | 213.22 | 201.53 | 0.93 |
| Admin C (240 μg BUD- | Mean | 1195.33 | 0.21 | 1183.45 | 1365.27 | 2.81 |
| SBE7-β-CD, Pari eFlow) | SD | 811.61 | 0.24 | 328.57 | 313.04 | 0.57 |
| Admin D (500 μg BUD, | Mean | 556.74 | 0.24 | 739.99 | 867.56 | 2.18 |
| Pari LC Plus) | SD | 193.63 | 0.25 | 220.09 | 216.95 | 1.1 |
| Admin E (1000 μg BUD, | Mean | 1114.83 | 0.23 | 1989.93 | 2083.5 | 2.33 |
| Pari LC Plus) | SD | 593.16 | 0.24 | 379.46 | 394.37 | 0.66 |

### Example 8

Table 6 provides the doses for the study medications used in the clinical study set forth in Example 8 (described in detail below). The study medications comprised two test formulations of inhaled Captisol-Enabled® Budesonide Inhalation Solution (CBIS) (Treatments A and B) and two reference formulations of inhaled budesonide suspensions (Pulmicort Respules®) (Treatments C and D), and the nebulizer devices used for the delivery to the lung of said study medications.

TABLE 6

| Treatment | Dose | Inhaler |
|---|---|---|
| A | 60 μg CBIS | Pari eFlow inhaler |
| | (60 μg Budesonide, 2% Captisol ®) | |
| B | 120 μg CBIS | Pari eFlow inhaler |
| | (120 μg Budesonide, 4% Captisol ®) | |
| C | 250 μg Pulmicort Respules ® | Pari LC Plus jet |
| | (250 μg Budesonide suspension) | nebulizer |
| D | 500 μg Pulmicort Respules ® | Pari LC Plus jet |
| | (500 μg Budesonide suspension) | nebulizer |

Individual components of the test and reference formulations are detailed in Table 7.

TABLE 7

| | | | Treatment B | Treatment C | Treatment D |
|---|---|---|---|---|---|
| | | Treatment A | Test | Reference | Reference |
| Component | Ingredient | 60 μg CBIS | 120 ug CBIS | 250 μg PR | 500 μg PR |
| Active | Budesonide | Approx | Approx | 250 μg/2 mL | 500 μg/2 mL |
| Ingredient | | 60 μg/0.5 mL | 120 μg/0.5 mL | | |
| Solubilizing | Captisol ® | 2% | 4% | None | None |
| Agent | | | | | |
| Chelating | EDTA | 0.01% | 0.01% | 0.01% | 0.01% |
| Agent | | | | | |
| Isotonicity | NaCl | 0.85% | 0.85% | 0.85% | 0.85% |
| Buffer(s) | Na Citrate | 0.05% | 0.05% | 0.05% | 0.05% |
| | Citric Acid | 0.03% | 0.03% | 0.03% | 0.03% |

### Example 9

Set forth in Table 8 is the summary of the pharmacokinetic profiles for budesonide following a single-centre, double-blind, multiple dose, parallel group, placebo controlled, two period crossover study involving Administrations A to D as set forth in Example 8. Forty-eight (48) healthy male volunteers were used in this clinical study. Each subject that qualified for the study was randomized to receive one of the following treatments: Treatment A (60 μg CBIS solution), Treatment B (120 μg CBIS solution), Treatment C (250 μg Pulmicort Respule Respule® suspension (250 μg Pulmicort)), Treatment D (500 μg Pulmicort Respule® suspension (500 μg Pulmicort)). The Subjects received Treatment A, B, C or D twice daily for seven days. Each subject received active drug and placebo crossed over two study periods. Table 8 provides the values for each of the pharmacokinetic parameters measured in the study for the administration of Treatments A-D during the clinical study.

TABLE 8

| | Summary | Cmax | Tmax | AUC₍ₗₐₛₜ₎ | AUC_{(0-INF)} | t½ |
|---|---|---|---|---|---|---|
| Dose | Statistic | (pg/ml) | (h) | (pg/ml · h) | (pg/ml · h) | (h) |
| Treatment A | N | 12 | 12 | 12 | 12 | 12 |
| (60 μg CBIS) | Mean | 402.001 | 0.117 | 310.861 | 424.875 | 2.4860 |
| | SD | 192.549 | 0.058 | 95.652 | 157.589 | 2.9550 |
| | Min | 186.370 | 0.08 | 106.380 | 156.520 | 0.7000 |
| | Median | 378.730 | 0.08 | 317.000 | 409.780 | 1.6800 |
| | Max | 779.340 | 0.25 | 463.070 | 748.450 | 11.7200 |
| | CV (%) | 47.898 | N/A | 30.800 | 37.100 | 118.9000 |
| | Geo. Mean | 362.202 | N/A | 293.699 | 396.119 | 1.8370 |
| Treatment B | N | 11 | 11 | 11 | 11 | 11 |
| (120 μg CBIS) | Mean | 625.337 | 0.14 | 726.658 | 860.632 | 2.4270 |
| | SD | 357.439 | 0.13 | 417.236 | 464.844 | 0.9530 |
| | Min | 169.800 | 0.08 | 168.220 | 221.420 | 1.5000 |
| | Median | 735.280 | 0.08 | 576.370 | 740.700 | 1.9800 |
| | Max | 1160.440 | 0.50 | 1496.720 | 1863.680 | 4.4000 |
| | CV (%) | 57.159 | N/A | 57.400 | 54.000 | 39.3000 |
| | Geo. Mean | 516.991 | N/A | 621.429 | 752.164 | 2.2840 |
| Treatment C | N | 11 | 11 | 11 | 11 | 11 |
| (250 μg Pulmicort) | Mean | 319.673 | 0.22 | 361.162 | 472.314 | 2.0230 |
| | SD | 185.337 | 0.22 | 212.378 | 239.042 | 0.9370 |
| | Min | 107.450 | 0.08 | 78.570 | 148.720 | 0.9100 |
| | Median | 231.560 | 0.08 | 318.120 | 425.030 | 1.7600 |
| | Max | 574.720 | 0.75 | 803.030 | 920.020 | 4.3200 |
| | CV (%) | 57.977 | N/A | 58.800 | 50.600 | 46.3000 |
| | Geo. Mean | 270.563 | N/A | 302.632 | 413.000 | 1.8600 |
| Treatment D | N | 12 | 12 | 12 | 12 | 12 |
| (500 μg Pulmicort) | Mean | 491.398 | 0.186 | 811.145 | 945.718 | 2.4930 |
| | SD | 207.942 | 0.193 | 328.022 | 363.252 | 0.7700 |
| | Min | 199.390 | 0.08 | 236.220 | 381.480 | 1.2100 |
| | Median | 474.310 | 0.08 | 856.320 | 952.860 | 2.4600 |
| | Max | 963.250 | 0.75 | 1224.800 | 1454.800 | 3.6600 |
| | CV (%) | 42.316 | N/A | 40.400 | 38.400 | 30.9000 |
| | Geo. Mean | 451.661 | N/A | 735.111 | 874.697 | 2.3720 |

### Example 10

The aqueous inhalation mixtures set forth in Examples 3 and 4 are delivered to a patient population according the methods set forth in Example 5. Pulmicort Respules® is likewise administered according the methods set forth in Example 5. The pharmacokinetic profile of the aqueous inhalation solutions will exhibit enhanced pharmacokinetic parameters as compared to the pharmacokinetic profile of the Pulmicort Respules®. For example, the aqueous inhalation solutions will display greater Cₘₐₓ, AUC₍ₗₐₛₜ₎, AUC_{(0-∞)} values and/or lower Tₘₐₓ values as compared to Pulmicort Respules®. Likewise, the aqueous inhalation solutions will display equal Cₘₐₓ, AUC₍ₗₐₛₜ₎, and AUC_{(0-∞)} values as compared to Pulmicort Respules® if administered at a lower dosage.

### Example 11

The aqueous inhalation mixtures set forth in Examples 1 and 2 are delivered to a patient population according the methods set forth in Example 5. Pulmicort Respules®, at a nominal dosages ranging from 1000 μg/dose up to 2500 μg/dose, are administered according the methods set forth in Example 5. The pharmacokinetic profile of the aqueous inhalation solutions will exhibit enhanced pharmacokinetic parameters as compared to the pharmacokinetic profile of the Pulmicort Respules®. For example, the aqueous inhalation solutions will display substantially equal Cₘₐₓ, (AUC₍ₗₐₛₜ₎, AUC_{(0-∞)} values as the Pulmicort Respules® even though the nominal dosages to be administered are substantially lower.

### Example 12

Preparation and use of an aqueous inhalation mixture containing a corticosteroid, a solubility enhancer and albuterol sulfate or levalbuterol HCl (Xopenex)

A citrate buffer (3 mM pH 4.5) is prepared as follows. Approximately 62.5 mg of citric acid is dissolved in and brought to volume with water in one 100 ml volumetric flask. Approximately 87.7 mg of sodium citrate is dissolved in and brought to volume with water in another 100 mL volumetric flask. In a beaker, the sodium citrate solution is added to the citric acid solution until the pH is approximately 4.5.

Approximately 10.4 mg of budesonide and 1247 mg of Captisol® (CyDex Inc.) are ground together with a mortar and pestle and transferred to a 10 mL flask. Buffer solution is added, and the mixture is vortexed, sonicated and an additional 1.4 mg budesonide is added. After shaking overnight, the solution is filtered through a 0.22 μm Durapore Millex-GV Millipore syringe filter unit. The resulting budesonide concentration is about 1 mg/ml. Approximately 0.5 ml of the budesonide solution is added to a unit dose of either Proventil® (2.5 mg/3 mL) or Xopenex® (1.25 mg/3 mL) thereby forming a clear aqueous inhalation mixture suitable for use in an inhalation nebulizer as described in Example 5.

### Example 13

Preparation and Use of an aqueous inhalation mixture containing a corticosteroid, a solubility enhancer, and formoterol (FORADIL®; (formoterol fumarate inhalation powder)).

The contents of one capsule containing 12 μg of formoterol fumarate blended with 25 mg of lactose is emptied into a vial to which is added 3 mL of 3 mM citrate buffer (pH 4.5) prepared as described in Example 12. The contents of the vial are vortexed to dissolve the solids present. The budesonide concentrate is prepared as described in Example 9 to provide a concentration of 1 mg/mL.

Approximately 1 ml of the budesonide solution is added to the formoterol fumarate buffered solution. The combination is a clear aqueous inhalation mixture suitable for use in an inhalation nebulizer as described in Example 5.

### Example 14

The aqueous inhalation mixtures set forth in Examples 12 and 13 are delivered to a patient population according the methods set forth in Example 5. Pulmicort Respules® is likewise administered according the methods set forth in Example 5. The pharmacokinetic profile of the aqueous inhalation mixtures will exhibit enhanced pharmacokinetic parameters as compared to the pharmacokinetic profile of the Pulmicort Respules®. For example, the aqueous inhalation solutions will display greater Cₘₐₓ, AUC₍ₗₐₛₜ₎, AUC_{(0-∞)} values and/or lower Tₘₐₓ values as compared to Pulmicort Respules® if administered at the same nominal dosage. Likewise, the aqueous inhalation solutions will display equal Cₘₐₓ, AUC₍ₗₐₛₜ₎, and AUC_{(0-∞)} values as compared to Pulmicort Respules® if administered at a lower nominal dosage.

### Example 15

Using traditional cascade impactor for in vitro testing of particle sizes, the following deposition characteristics are observed for both a budesonide solution and a Pulmicort Respule suspension. The budesonide solution was nebulized using a PARI eFlow device. The Pulmicort Respule suspension was nebulized using a PARI LC Plus nebulizer. These results are further transformed in pulmonary deposition efficiencies are shown in Table 9, using different definitions of pulmonary deposition as function of the stage range (Stage 3-7, Stage 4-7, Stage 5-7). Table 10 shows that dependent on the stage range used for pulmonary deposition (stage 3-7, stage 4-7, stage 5-7) the ratio of pulmonary deposition (expressed as the ratio of eFlow/Pari LC plus depositions) ranges from 1.2, 1.9 to 3.8 for stage 3-7, stage 4-7 or stage 5-7, respectively. Fine particle fraction is defined as particle sizes less than 4.7 μm (Bosco A P et al., In Vitro Estimation of In Vivo Jet Nebulizer Efficiency Using Actual and Simulated Tidal Breathing Patterns, Journal of Aerosol Medicine 18(4): 427-38 (2005); herein incorporated by reference in its entirety).

TABLE 9

| | | Stage Deposition | Pari LC Plus |
|---|---|---|---|
| Stage | Size | eFLOW with Budesonide Solution | with Pulmicort |
| Number | μm | % | % |
| 3 | 5.36 | 5 | 32 |
| 4 | 3.3 | 20 | 28 |
| 5 | 2.08 | 40 | 13 |
| 6 | 1.36 | 24 | 4 |
| 7 | 0.95 | 5 | 1 |
| Filter | <0.95 | 3 | 1 |

TABLE 10

| | | % Pulmonary Deposition | | |
|---|---|---|---|---|
| | Size Range | eFLOW with | Pari LC Plus | |
| Stage Range | μm | Budesonide Solution | with Pulmicort | Ratio |
| Stage 3-7 | 5.36-0.98 | 94 | 78 | 1.2 |
| Stage 4-7 | 3.3-0.98 | 89 | 46 | 1.9 |
| Stage 5-7 | 2.08-0.98 | 69 | 18 | 3.8 |

*f = filter = 0.98 μm

### Example 16

The rate at which the budesonide concentration increased over the course of a three minute nebulization period within a PARI LC-PLUS nebulizer was determined for Captisol-Budesonide Inhalation Solution (CBIS) formulations (budesonide concentration of 60 μg/0.5 ml) and Pulmicort Respules (PR) formulations (budesonide concentration of 250 μ/g/2.0 ml). Samples for both formulations were pooled separately and three samples of 1 ml were taken from each pool to get the concentrations at t=0 for both the Captisol-Budesonide Inhalation Solution (CBIS) formulations and Pulmicort Respules (PR) formulations. Three separate experiments were performed for each time point to avoid concentration changes due to sample collection: t=1 (termination of nebulization after 1 min. (n=3, for CBIS and PR), a sample of 1 ml was taken from the LC-PLUS nebulizer; t=2 (termination of nebulization after 2 min. (n=3, for CBIS and PR), a sample of 1 ml was taken from the LC-PLUS nebulizer; t=3 (termination of nebulization after 3 min. (n=3, for CBIS and PR), a sample of 1 ml was taken from the LC-PLUS nebulizer.

### Example 17

Set forth in Tables 11 and 12 are the results of the determination of the rate increase in concentration of Captisol-Budesonide Inhalation Solution (CBIS) formulations and Pulmicort Respules (PR) formulations inside a PARI LC-Plus nebulizer over the course of a three minute nebulization period as determined by the methods of Example 16. Three run times lasting three minutes were conducted for both the CBIS (CBIS 1-3) and the Pulmicort Respules (PR 1-3). All CBIS solutions had a budesonide concentration of 60 μg/0.5 ml. All Pulmicort Respule formulations had a budesonide concentration of 250 μg/2.0 ml.

TABLE 11

| Sample (%) | t = 0 | t = 1 min | t = 2 min | t = 3 min |
|---|---|---|---|---|
| CBIS 1 | 100 | 100 | 103 | 108 |
| CBIS 2 | 100 | 101 | 106 | 109 |
| CBIS 3 | 100 | 101 | 105 | 109 |
| Mean | 100 | 101 | 105 | 108 |
| SD | 0.0 | 0.7 | 1.4 | 0.6 |
| PR 1 | 100 | 100 | 105 | 119 |
| PR 2 | 100 | 101 | 109 | 123 |
| PR 3 | 100 | 97 | 102 | 122 |
| Mean | 100 | 99 | 105 | 121 |
| SD | 0.0 | 2.4 | 3.4 | 2.2 |

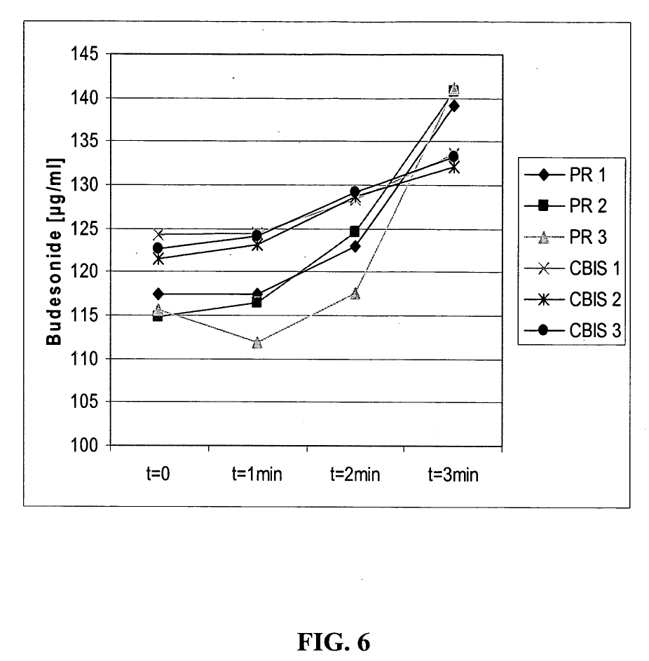

Table 11 shows the concentration for CBIS formulations 1-3 and PR formulations 1-3 inside the nebulizer device over the course of the three minute run time with measurement taken at 0, 1, 2, and 3 minutes. As provided in Table 11, the concentration of CBIS formulations 1-3 and PR formulations 1-3 inside the nebulizer device is 100% at the beginning of the run (t=0 min). After the three minute nebulization period (t=3 min), the concentration of the CBIS formulations inside the nebulizer device increased to an average of 108% of the starting concentration. During an identical nebulization period (t=3 min), the concentration of the Pulmicort Respule formulations inside the nebulizer device increased to an average of 121% of the starting concentration. FIG. 6 provides a graphical representation of the data provided in Table 11.

TABLE 12

| Sample (μg/ml) | t = 0 | t = 1 min | t = 2 min | t = 3 min |
|---|---|---|---|---|
| CBIS 1 | 124.24 | 124.43 | 128.30 | 133.65 |
| CBIS 2 | 121.50 | 123.10 | 128.63 | 132.09 |
| CBIS 3 | 122.66 | 124.19 | 129.16 | 133.18 |
| Mean | 122.80 | 123.91 | 128.7 | 132.97 |
| SD | 1.38 | 0.71 | 0.43 | 0.80 |
| PR 1 | 117.40 | 117.44 | 123.02 | 139.20 |
| PR 2 | 114.84 | 116.50 | 124.68 | 140.77 |
| PR 3 | 115.68 | 111.92 | 117.65 | 141.12 |
| Mean | 115.97 | 115.29 | 121.78 | 140.36 |
| SD | 1.30 | 2.95 | 3.67 | 1.02 |

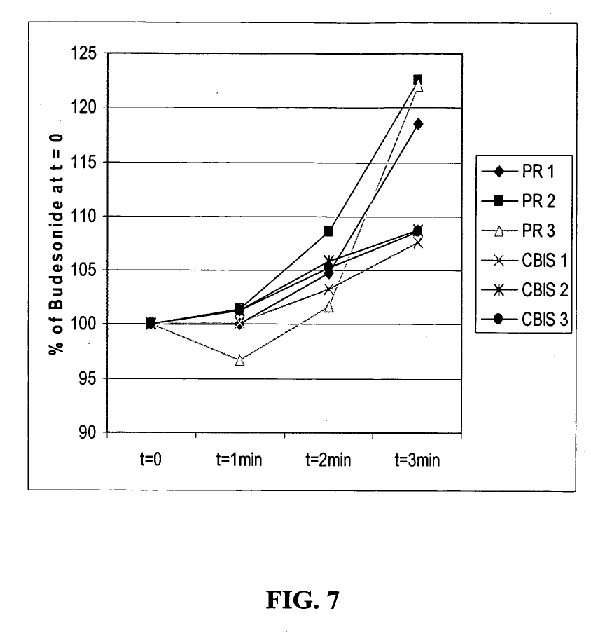

Table 12 shows the concentration for CBIS formulations 1-3 and PR formulations 1-3 inside the nebulizer device over the course of the three minute run time with measurement taken at 0, 1, 2, and 3 minutes. As provided in Table 12, the average concentration of CBIS formulations 1-3 inside the nebulizer device is 122.8 μg/ml at the beginning of the run (t=0 min). The average concentration of PR formulations 1-3 inside the nebulizer device is 115.97 μg/ml at the beginning of the run (t=0 min). After the three minute nebulization period (t=3 min), the average concentration of the CBIS formulations inside the nebulizer device increased to 132.97 μg/ml. During an identical nebulization period (t=3 min), the average concentration of the Pulmicort Respule formulations inside the nebulizer device increased to 140.36 μg/ml. FIG. 7 provides a graphical representation of the data provided in Table 11.

What is claimed is:

1. An inhalable composition comprising about 250 μg or less of a single corticosteroid, a solvent and a solubility enhancer, wherein upon administration of the composition to a subject through a device, the composition achieves a rate of increasing concentration of the corticosteroid inside the device of about 5 μg/ml per minute or less over administration of the corticosteroid through the device, and wherein the composition is substantially free of active pharmaceutical agents other than the corticosteroid.

2. The composition of claim 1, wherein the composition achieves a rate of increasing concentration of the corticosteroid inside the device of about 5 μg/ml per minute or less over the first three minutes of administration.

3. The composition of claim 1, wherein the composition achieves a rate of increasing concentration of the single corticosteroid inside the device of about 3.5 μg/ml per minute or less over the first 3 minutes of administration.

4. The composition of claim 1, wherein the composition achieves a rate of increase in concentration of the single corticosteroid inside the device of about 5% per minute or less over the first 3 minutes of administration.

5. The composition of claim 1, wherein the solvent comprises water.

6. The composition of claim 1, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

7. The composition of claim 1, wherein the solubility enhancer comprises SBE7-β-CD.

8. The composition of claim 1, wherein the device is a nebulizer.

9. The composition of claim 1, wherein the device is a Pari eFlow nebulizer.

10. The composition of claim 1, wherein the composition comprises the single corticosteroid in a nominal dosage of about 60 μg.

11. The composition of claim 1, wherein the composition comprises the single corticosteroid in a nominal dosage of about 120 μg.

12. An inhalable composition comprising an effective amount of a single corticosteroid, a solvent and a solubility enhancer, wherein upon administration of the composition to a subject through a device, the composition achieves a rate of increasing concentration of the corticosteroid inside the device of 60% or less of a rate of increasing concentration of the corticosteroid inside the device achieved by an inhalable suspension comprising the corticosteroid without a solubility enhancer administered under the same conditions, and wherein the composition is substantially free of active pharmaceutical agents other than the corticosteroid.

13. The composition of claim 12, wherein the rate of increasing concentration of the single corticosteroid inside the device is achieved over the first 3 minutes, during the second and third minutes or during the third minute of administration.

14. The composition of claim 12, wherein administration of the composition through the device is achieved over five minutes or less, and administration of the inhalable suspension is achieved over five minutes or less.

15. The composition of claim 14, wherein the time of administration of the composition through the device and the time of administration of the inhalable suspension are the same.

16. The composition of claim 14, wherein the time of administration of the composition through the device and the time of administration of the inhalable suspension are different.

17. The composition of claim 12, wherein the composition comprises about 40, about 60, about 120, about 125, about 240, about 250, about 500, about 1000, about 1500, or about 2000 μg of the corticosteroid.

18. The composition of claim 12, wherein the solvent comprises water.

19. The composition of claim 12, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

20. The composition of claim 12, wherein the solubility enhancer comprises SBE7-β-CD.

21. The composition of claim 12, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

22. A method of generating fine particles from an inhalable composition comprising:
forming the composition by adding a solvent and a solubility enhancer to an effective amount of a single corticosteroid, wherein the composition is substantially free of active pharmaceutical agents other than the corticosteroid; and
operating a nebulizer to produce fine particles of the composition,
wherein upon administration of the composition to a subject through the nebulizer, the composition achieves a rate of increasing concentration of the corticosteroid inside the nebulizer of 60% or less of a rate of increasing concentration of the corticosteroid inside the nebulizer achieved by an inhalable suspension comprising the corticosteroid without a solubility enhancer administered under the same conditions.

23. The method of claim 22, wherein the rate of increasing concentration of the single corticosteroid inside the device is achieved over the first 3 minutes, during the second and third minutes or during the third minute of administration.

24. The method of claim 22, wherein administration of the composition through the nebulizer is achieved over five minutes or less, and administration of the inhalable suspension is achieved over five minutes or less.

25. The method of claim 24, wherein the time of administration of the composition through the nebulizer and the time of administration of the inhalable suspension are the same.

26. The method of claim 24, wherein the time of administration of the composition through nebulizer and the time of administration of the inhalable suspension are different.

27. The method of claim 22, wherein the composition comprises about 40, about 60, about 120, about 125, about 240, about 250, about 500, about 1000, about 1500, or about 2000 μg of the single corticosteroid.

28. The method of claim 22, wherein the solvent comprises water.

29. The method of claim 22, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

30. The method of claim 29, wherein the solubility enhancer comprises SBE7-β-CD.

31. The method of claim 22, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

32. An inhalation system for delivering a therapeutically effective dose of a single corticosteroid to a patient comprising:
(a) an aqueous inhalation mixture comprising the corticosteroid, a solvent and a solubility enhancer, wherein the composition is substantially free of active pharmaceutical agents other than the corticosteroid and
(b) a nebulizer
wherein upon administration of the mixture to a subject through the nebulizer, the mixture achieves a rate of increasing concentration of the corticosteroid inside the nebulizer of 60% or less of a rate of increasing concentration of the corticosteroid inside the nebulizer achieved by an inhalable suspension comprising the corticosteroid without a solubility enhancer administered under the same conditions.

33. The system of claim 32, wherein the rate of increasing concentration of the single corticosteroid inside the device is achieved over the first 3 minutes, during the second and third minutes or during the third minute of administration.

34. The system of claim 32, wherein administration of the composition through the nebulizer is achieved over five minutes or less, and administration of the inhalable suspension is achieved over five minutes or less.

35. The system of claim 34, wherein the time of administration of the composition through the nebulizer and the time of administration of the inhalable suspension are the same.

36. The system of claim 34, wherein the time of administration of the composition through the nebulizer and the time of administration of the inhalable suspension are different.

37. The system of claim 32, wherein the mixture comprises about 40, about 60, about 120, about 125, about 240, about 250, about 500, about 1000, about 1500, or about 2000 μg of the single corticosteroid.

38. The system of claim 32, wherein the solvent comprises water.

39. The system of claim 32, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

40. The system of claim 39, wherein the solubility enhancer comprises SBE7-β-CD.

41. The system of claim 32, wherein the nebulizer is selected from jet nebulizers, ultrasonic nebulizers, pulsating membrane nebulizers, nebulizers with a vibrating mesh or plate with multiple apertures, and nebulizers comprising a vibration generator and an aqueous chamber.

42. A method for the treatment of a bronchoconstrictive disorder in a patient in need of treatment thereof, comprising:
forming a composition by adding a solvent and a solubility enhancer to an amount of a single corticosteroid, wherein the composition is substantially free of active pharmaceutical agents other than the corticosteroid; and
operating a nebulizer,
wherein upon administration of the composition to a subject through the nebulizer, the composition achieves a rate of increasing concentration of the corticosteroid inside the nebulizer of 60% or less of a rate of increasing concentration of the corticosteroid inside the nebulizer achieved by an inhalable suspension comprising the corticosteroid without a solubility enhancer administered under the same conditions.

43. The method of claim 42, wherein the rate of increasing concentration of the single corticosteroid inside the device is achieved over the first 3 minutes, during the second and third minutes or during the third minute of administration.

44. The method of claim 42, wherein administration of the composition through the nebulizer is achieved over five minutes or less, and administration of the inhalable suspension is achieved over five minutes or less.

45. The method of claim 44, wherein the time of administration of the composition through the nebulizer and the time of administration of the inhalable suspension are the same.

46. The method of claim 44, wherein the time of administration of the composition through the nebulizer and the time of administration of the inhalable suspension are different.

47. The method of claim 42, wherein the composition comprises about 40, about 60, about 120, about 125, about 240, about 250, about 500, about 1000, about 1500, or about 2000 μg of the single corticosteroid.

48. The method of claim 42, wherein the solvent comprises water.

49. The method of claim 42, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

50. The method of claim 49, wherein the solubility enhancer comprises SBE7-β-CD.

51. The method of claim 42, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

52. The method of claim 42, wherein the bronchoconstrictive disorder is selected from the group consisting of asthma, pediatric asthma, bronchial asthma, allergic asthma, intrinsic asthma, chronic obstructive pulmonary disease (COPD), chronic bronchitis, and emphysema.

53. An inhalable composition comprising about 250 μg or less of budesonide, a solvent and a solubility enhancer, wherein upon administration of the composition to a subject through a device, the composition achieves a rate of increasing concentration of budesonide inside the device of about 5 μg/ml per minute or less over administration of the budesonide through the device, and wherein the composition is substantially free of active pharmaceutical agents other than the corticosteroid.

54. The composition of claim 53, wherein the composition achieves a rate of increasing concentration of the budesonide inside the device of about 5 μg/ml per minute or less over the first three minutes of administration.

55. The composition of claim 54, wherein the composition achieves a rate of increasing concentration of budesonide inside the device of about 3.5 μg/ml per minute or less over the first 3 minutes of administration.

56. The composition of claim 53, wherein the composition achieves a rate of increase in concentration of budesonide inside the device of about 5% per minute or less over the first 3 minutes of administration.

57. The composition of claim 53, wherein the solvent comprises water.

58. The composition of claim 53, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

59. The composition of claim 58, wherein the solubility enhancer comprises SBE7-β-CD.

60. The composition of claim 53, wherein the device is a nebulizer.

61. The composition of claim 60, wherein the device is a Pari eFlow nebulizer.

62. The composition of claim 53, wherein the composition comprises budesonide in a nominal dosage of about 60 μg.

63. The composition of claim 53, wherein the composition comprises budesonide in a nominal dosage of about 120 μg.

64. An inhalable composition comprising an effective amount of budesonide, a solvent and a solubility enhancer, wherein upon administration of the composition to a subject through a device, the composition achieves a rate of increasing concentration of budesonide inside the device of 60% or less of a rate of increasing concentration of budesonide inside the device achieved by an inhalable suspension comprising budesonide without a solubility enhancer administered under the same conditions, and wherein the composition is substantially free of active pharmaceutical agents other than the corticosteroid.

65. The composition of claim 64, wherein the rate of increasing concentration of budesonide inside the device is achieved over the first 3 minutes, during the second and third minutes or during the third minute of administration.

66. The composition of claim 64, wherein administration of the composition through the device is achieved over five minutes or less, and administration of the inhalable suspension is achieved over five minutes or less.

67. The composition of claim 66, wherein the time of administration of the composition through the device and the time of administration of the inhalable suspension are the same.

68. The composition of claim 66, wherein the time of administration of the composition through the device and the time of administration of the inhalable suspension are different.

69. The composition of claim 64, wherein the composition comprises about 40, about 60, about 120, about 125, about 240, about 250, about 500, about 1000, about 1500, or about 2000 μg of budesonide.

70. The composition of claim 64, wherein the solvent comprises water.

71. The composition of claim 64, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

72. The composition of claim 71, wherein the solubility enhancer comprises SBE7-β-CD.

73. The composition of claim 64, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

74. A method of generating fine particles from an inhalable composition comprising:
forming the composition by adding a solubility enhancer to an effective amount of budesonide, wherein the composition is substantially free of active pharmaceutical agents other than the budesonide; and
operating a nebulizer to produce fine particles of the composition,
wherein upon administration of the composition to a subject through the nebulizer, the composition achieves a rate of increasing concentration of budesonide inside the nebulizer of 60% or less of a rate of increasing concentration of budesonide inside the nebulizer achieved by an inhalable suspension comprising budesonide without a solubility enhancer administered under the same conditions.

75. The method of claim 74, wherein the rate of increasing concentration of budesonide inside the device is achieved over the first 3 minutes, during the second and third minutes or during the third minute of administration.

76. The method of claim 74, wherein administration of the composition through the nebulizer is achieved over five minutes or less, and administration of the inhalable suspension is achieved over five minutes or less.

77. The method of claim 76, wherein the time of administration of the composition through the nebulizer and the time of administration of the inhalable suspension are the same.

78. The method of claim 76, wherein the time of administration of the composition through nebulizer and the time of administration of the inhalable suspension are different.

79. The method of claim 74, wherein the composition comprises about 40, about 60, about 120, about 125, about 240, about 250, about 500, about 1000, about 1500, or about 2000 μg of budesonide.

80. The method of claim 74, wherein the solvent comprises water.

81. The method of claim 74, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

82. The method of claim 81, wherein the solubility enhancer comprises SBE7-β-CD.

83. The method of claim 74, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

84. An inhalation system for delivering a therapeutically effective dose of budesonide to a patient comprising:
(a) an aqueous inhalation mixture comprising budesonide and a solubility enhancer, wherein the composition is substantially free of active pharmaceutical agents other than the corticosteroid; and
(b) a nebulizer
wherein upon administration of the mixture to a subject through the nebulizer, the mixture achieves a rate of increasing concentration of budesonide inside the nebulizer of 60% or less of a rate of increasing concentration of budesonide inside the nebulizer achieved by an inhalable suspension comprising budesonide without a solubility enhancer administered under the same conditions.

85. The system of claim 84, wherein the rate of increasing concentration of budesonide inside the device is achieved over the first 3 minutes, during the second and third minutes or during the third minute of administration.

86. The system of claim 84, wherein administration of the composition through the nebulizer is achieved over five minutes or less, and administration of the inhalable suspension is achieved over five minutes or less.

87. The system of claim 86, wherein the time of administration of the composition through the nebulizer and the time of administration of the inhalable suspension are the same.

88. The system of claim 86, wherein the time of administration of the composition through the nebulizer and the time of administration of the inhalable suspension are different.

89. The system of claim 84, wherein the mixture comprises about 40, about 60, about 120, about 125, about 240, about 250, about 500, about 1000, about 1500, or about 2000 μg of budesonide.

90. The system of claim 84, wherein the solvent comprises water.

91. The system of claim 84, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl--γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

92. The system of claim 91, wherein the solubility enhancer comprises SBE7-β-CD.

93. The system of claim 84, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

94. A method for the treatment of a bronchoconstrictive disorder in a patient in need of treatment thereof, comprising:
forming a composition by adding a solubility enhancer to an amount of budesonide, wherein the composition is substantially free of active pharmaceutical agents other than the budesonide; and
operating a nebulizer,
wherein upon administration of the composition to a subject through the nebulizer, the composition achieves a rate of increasing concentration of budesonide inside the nebulizer of 60% or less of a rate of increasing concentration of budesonide inside the nebulizer achieved by an inhalable suspension comprising budesonide without a solubility enhancer administered under the same conditions.

95. The method of claim 94, wherein the rate of increasing concentration of budesonide inside the device is achieved over the first 3 minutes, during the second and third minutes or during the third minute of administration.

96. The method of claim 94, wherein administration of the composition through the nebulizer is achieved over five minutes or less, and administration of the inhalable suspension is achieved over five minutes or less.

97. The method of claim 96, wherein the time of administration of the composition through the nebulizer and the time of administration of the inhalable suspension are the same.

98. The method of claim 96, wherein the time of administration of the composition through the nebulizer and the time of administration of the inhalable suspension are different.

99. The method of claim 94, wherein the composition comprises about 40, about 60, about 120, about 125, about 240, about 250, about 500, about 1000, about 1500, or about 2000 μg of budesonide.

100. The method of claim 94, wherein the solvent comprises water.

101. The method of claim 94, wherein the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE 1-β-CD, SBE4-β-CD, SBE7-β-CD, SBE-γ-CD, dimethyl β-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

102. The method of claim 101, wherein the solubility enhancer comprises SBE7-β-CD.

103. The method of claim 94, wherein the device is selected from jet nebulizers, ultrasonic nebulizers, pulsating membrane nebulizers, nebulizers with a vibrating mesh or plate with multiple apertures, and nebulizers comprising a vibration generator and an aqueous chamber.

104. The method of claim 94, wherein the bronchoconstrictive disorder is selected from the group consisting of asthma, pediatric asthma, bronchial asthma, allergic asthma, intrinsic asthma, chronic obstructive pulmonary disease (COPD), chronic bronchitis, and emphysema.
